(19) United States
(12) Patent Application Publication
(10) Pub. No.: US 2013/0022592 A1
(43) Pub. Date: Jan. 24, 2013

(54) Continuous subcutaneous insulin infusion methods with a hyaluronan-degrading enzyme

(71) Applicants: Daniel Edward Vaughn, Encinitas, CA (US); Douglas Boyer Muchmore, La Jolla, CA (US); Gregory I. Frost, Del Mar, CA (US)

(72) Inventors: Daniel Edward Vaughn, Encinitas, CA (US); Douglas Boyer Muchmore, La Jolla, CA (US); Gregory I. Frost, Del Mar, CA (US)

(21) Appl. No.: 13/507,261

(22) Filed: Jun. 15, 2012

Related U.S. Application Data
(60) Provisional application No. 61/628,389, filed on Oct. 27, 2011.
(60) Provisional application No. 61/520,940, filed on Jun. 17, 2011.
(60) Provisional application No. 61/657,606, filed on Jun. 8, 2012.

(51) Int. Cl. A61K 38/47; A61P 3/10; A61M 5/142; A61K 38/28

(52) U.S. Cl. 424/94.62; 514/6.1; 604/506

(57) ABSTRACT

Provided are methods for continuous subcutaneous insulin infusion (CSII) that employ a hyaluronan-degrading enzyme, including a recombinant human PH20 (rHuPH20). The methods can be used to more consistently control blood glucose during the course of CSII. The methods can be used to treat subjects having diabetes or other insulin-associated disease or condition.

### RELATED APPLICATIONS

Benefit of priority is claimed to U.S. provisional Application No. 61/628,389 filed Oct. 27, 2011, U.S. provisional Application No. 61/520,940 filed Jun. 17, 2011, and to U.S. provisional Application No. 61/657,606 filed Jun. 8, 2012, each entitled “Continuous Subcutaneous Insulin Infusion Methods With a Hyaluronan-Degrading Enzyme.”

This application is related to International Application Serial No. (Attorney Docket No. 33320.03097. WO01/3097PC), filed the same day herewith, entitled “CONTINUOUS SUBCUTANEOUS INSULIN INFUSION METHODS WITH A HYALURONAN-DEGRADING ENZYME,” which claims priority to U.S. provisional Application No. 61/628,389, U.S. provisional Application No. 61/520,940, and U.S. provisional Application No. 61/657,606. The subject matter of each of the above-noted related applications is incorporated by reference in its entirety.

This application also is related to provisional Application No. 61/520,962, filed Jun. 17, 2011, entitled “Stable Co-formulations of a Hyaluronan-Degrading Enzyme and Insulin.” This application also is related to U.S. application Ser. No. ______ (Attorney Docket No. 33320.03085. US01/3085) and to U.S. application Ser. No. ______ (Attorney Docket No. 33320.03085. US02/3085B), each filed the same day herewith, entitled “STABLE FORMULATIONS OF A HYALURONAN-DEGRADING ENZYME,” which claim priority to U.S. Provisional Application No. 61/520,962. This application also is related to International PCT Application No. (Attorney Docket No. 33320.03085. WP01/3085PC), filed the same day herewith, entitled “STABLE FORMULATIONS OF A HYALURONAN-DEGRADING ENZYME,” which claims priority to U.S. Provisional Application No. 61/520,962.

This application also is related to application Ser. No. 12/387,225, published as U.S. publication No. US20090304665, to Inventors Gregory Frost, Igor Blinsky, Daniel Vaughn and Barry Sugarman, entitled “Super Fast-Acting Insulin Compositions,” filed Apr. 28, 2009, which claims priority to U.S. Provisional Application No. 61/125,835, filed Apr. 28, 2008.

The subject matter of each of the above-noted applications is incorporated by reference in its entirety.

An electronic version on compact disc (CD-R) of the Sequence Listing is filed herewith in duplicate (labeled Copy #1 and Copy #2), the contents of which are incorporated by reference in their entirety. The computer-readable file on each of the aforementioned compact discs, created on Jun. 15, 2012, is identical, 860 kilobytes in size, and titled 3097seq.001.txt.

### FIELD OF THE INVENTION

Provided are methods for continuous subcutaneous insulin infusion (CSII) that employ a hyaluronan-degrading enzyme, such as a recombinant human PH20 (rHuPH20). The methods can be used to more consistently control blood glucose during the course of CSII. The methods can be used to treat subjects having diabetes or other insulin-associated disease or condition.

### BACKGROUND

Diabetes results in chronic hyperglycemia due to the inability of the pancreas to produce adequate amounts of insulin or due to the inability of cells to synthesize and release the insulin appropriately. Hyperglycemia also can be experienced by critically ill patients, resulting in increased mortality and morbidity. Insulin has been administered as a therapeutic to treat patients having diabetes, including, for example, type 1 diabetes, type 2 diabetes and gestational diabetes. Insulin also has been administered to critically ill patients with hyperglycemia to control blood glucose level. Typically, fast-acting insulins are administered to such subjects in response to hyperglycemia or in anticipation of hyperglycemia, such as following consumption of a meal, which can result in glycemic control. However, current fast-acting forms of insulins have a delay in absorption and action, and therefore do not approximate the rapid endogenous insulin action. Thus, such formulations do not act quickly enough to shut off hepatic glucose production that occurs shortly after this first phase of insulin release. Due to the delay in pharmacological action, the fast-acting insulin preparations should be administered in advance of meals in order to achieve the desired glycemic control. Further, the doses that must be administered lead to an extended duration of action that contributes to hypoglycemia, and in many cases, obesity. Thus, there exists a need for improved methods of insulin therapy to control blood glucose levels in diabetic subjects.

### SUMMARY

Provided are methods, compositions and uses for controlling blood glucose in a subject treated by continuous subcutaneous insulin infusion (CSII) therapy. Typically the subjects to be treated have diabetes, such as, but not limited to, type 1 diabetes mellitus, type 2 diabetes mellitus and gestational diabetes.

The methods provided herein include administering to the subject a composition containing a hyaluronan-degrading enzyme in a therapeutically effective amount sufficient to catalyze the hydrolysis of hyaluronic acid to increase tissue permeability; and performing CSII therapy to deliver a composition comprising a fast-acting insulin to the subject. Administration of the hyaluronan degrading enzyme generally is administered separately from the CSII therapy. For example, the hyaluronan degrading enzyme is administered prior to administration of CSII therapy by leading edge. To practice these methods, the hyaluronan-degrading enzyme is provided in an amount that effects an ultra-fast insulin response at the outset of CSII device's infusion set life. The methods can correct changes or differences in insulin absorption and/or action observed during CSII therapy, that is minimized or reduced over the course of infusion set life.

For example, provided herein is a method of controlling blood glucose in a subject by continuous subcutaneous insulin infusion (CSII) therapy by administering a composition containing a hyaluronan-degrading enzyme to the subject; and then continuously infusing a fast-acting insulin by CSII to the subject, wherein the difference in insulin absorption is minimized or reduced over the course of infusion set life compared to CSII performed in the absence of the hyaluronan-degrading enzyme. In examples of the methods herein, the hyaluronan-degrading enzyme can be administered in an amount that effects an ultra-fast insulin response at the outset of infusion set life in the subject. In other examples herein, the hyaluronan-degrading enzyme can be administered in an amount sufficient to catalyze the hydrolysis of hyaluronic acid to increase tissue permeability.

To practice the methods, the CSII therapy is effected with a continuous infusion device that includes an insulin pump, a reservoir containing the fast-acting insulin, an optional glucose monitor, and an infusion set for subcutaneous infusion of the composition. In some examples, the CSII therapy is effected with a continuous infusion device that includes an insulin pump, a reservoir containing the fast-acting insulin, a glucose monitor, and an infusion set for subcutaneous infusion of the composition. The continuous infusion device can provide an open-loop or closed-loop system.

In practicing the methods, the CSII therapy step, is performed or continues for a predetermined time. Typically, the hyaluronan-degrading enzyme composition is administered before infusion of the fast-acting insulin. The hyaluronan-degrading enzyme composition can be administered, before, after or during the first interval or simultaneously with commencing the first interval. The hyaluronan-degrading enzyme composition is periodically reinfused. Typically CSII therapy is performed for a predetermined interval; and at beginning of each interval, the hyaluronan degrading enzyme composition is administered. At the end of each interval the infusion set (or the entire pump) can be replaced. Typical predetermined interval generally are more than a day, several days, such as 2 days to 4 days, or can be longer, such as a week.

The hyaluronan-degrading enzyme can be administered at or near the site of infusion of the insulin composition of the CSII device, including through the same injection site or different injection sites. The hyaluronan-degrading enzyme and the fast-acting insulin composition can be administered sequentially, simultaneously or intermittently. The hyaluronan degrading enzyme generally is to be administered prior to commencing the CSII therapy or when changing the CSII device or injection set. Hence, the hyaluronan-degrading enzyme is administered prior to insulin in any interval of CSII therapy. The delivery of the hyaluronan degrading enzyme composition can be administered immediately prior to initiation of infusion by CSII or when or before a CSII set begins. For example, the insulin infusion can be initiated within seconds or minutes of administration of the hyaluronan-degrading enzyme. In some instances, the hyaluronan-degrading enzyme is administered at least one hour before initiation of the insulin infusion, such as at least 2 hours. For example, the hyaluronan-degrading enzyme generally is administered about or approximately or 15 seconds to 1 hour prior to insulin infusion, 30 seconds to 30 minutes, 1 minute to 15 minutes, 1 minute to 12 hours, 5 minutes to 6 hours, 30 minutes to 3 hours, or 1 hour to 2 hours prior to commencement of CSII. In some examples, the delivery of the hyaluronan degrading enzyme can be after CSII therapy, such as 1 minute to 12 hours, 5 minutes to 6 hours, 30 minutes to 3 hours, or 1 hour to 2 hours after. Since CSII therapy typically is continuous, the hyaluronidase degrading enzyme will be administered at predetermined intervals with therapy or can be administered as needed if changes or difference in insulin absorption and/or action are observed during CSII therapy. Typically, the hyaluronan-degrading enzyme is administered no more than 2 hours before administration of the fast-acting insulin.

In these methods, the amount of hyaluronan-degrading enzyme administered is functionally equivalent to between or about between 1 Unit to 200 Units, 5 Units to 150 Units, 10 Units to 150 Units, 50 Units to 150 Units or 1 Unit to 50 Units of the enzyme. For example, the amount of hyaluronan-degrading enzyme administered is typically between 8 ng to 2 μg, 20 ng to 1.6 μg, 80 ng to 1.25 μg or 200 ng to 1 μg, particularly of the enzyme produced by expression of nucleic acid that encodes amino acids 36-482 in CHO cells or equivalent amounts of other hyaluronidase degrading enzymes.

Also provided are continuous subcutaneous insulin infusion (CSII) dosage regimens for controlling blood glucose, particularly in subjects treated with co-formulations of insulin and a hyaluronan degrading enzyme (e.g. a super-fast acting insulin composition). In accord with these regimens, extra insulin can be periodically administered in order to counteract any decrease in level or action or increase in blood glucose that occurs when co-formulation of fast-acting insulins with a hyaluronan degrading enzyme, and optional basal insulin, are administered.

The methods are practiced by: a) performing CSII to deliver a composition containing a super fast-acting insulin composition to a subject in accord with a programmed basal rate and bolus dose of insulin; and b) at least once during the course of treatment, increasing the amount of basal insulin and/or bolus insulin administered by at least 1% compared to the programmed basal rate and bolus dose of insulin administered in the absence of a hyaluronan-degrading enzyme thereby increasing insulin action. Step b) can be performed at least once per day. In some embodiments, the basal insulin rate is increased, and in others the bolus dose of insulin is increased, and in other the basal insulin and bolus dose are increased. For these regimens, the bolus dose can be the prandial dose for a given mean and/or the correction bolus for a given hyperglycemic correction. The basal rate and/or bolus dose can be increased 1% to 50%, 5% to 40%, 10% to 20% or 5% to 15%.

For these regimens and any methods in which a super-fast acting insulin composition (that contains a fast-acting insulin and an hyaluronidase degrading enzyme) is administered, such super-fasting acting insulin composition contains: a therapeutically effective amount of a fast-acting insulin for controlling blood glucose levels; and an amount of a hyaluronan-degrading enzyme sufficient to render the composition a super fast-acting insulin composition. Exemplary of compositions are those where: the amount of fast-acting insulin is from or from about 10 U/mL to 1000 U/mL; and the sufficient amount of a hyaluronan-degrading enzyme to render the composition super fast-acting is functionally equivalent to 1 U/mL to 10,000 U/mL, such as, for example, where the amount of a fast-acting insulin is or is about 100 U/mL, and the sufficient amount of a hyaluronan-degrading enzyme to render the composition super fast-acting is functionally equivalent to or about to 600 U/mL; a composition where the amount of fast-acting insulin is from or from about 0.35 mg/mL to 35 mg/mL; and the sufficient amount of a hyaluronan-degrading enzyme to render the composition super fast-acting is functionally equivalent to 8 ng/mL to 80 μg/mL.

In all methods provided herein the hyaluronan-degrading enzyme can be a hyaluronidase or a chondroitinase. The hyaluronan-degrading enzyme can be a hyaluronidase that is active at neutral pH. In some embodiments, the hyaluronan-degrading enzyme lacks a glycosylphosphatidylinositol (GPI) anchor or is not membrane-associated when expressed from a cell, such as an hyaluronidase degrading enzyme that lacks a GPI anchor, or one that normally has a GPI anchor, but has C-terminal truncations of one or more amino acid residues to remove all or part of a GPI anchor. Hyaluronidase degrading enzymes include a hyaluronidase that is a PH20, such as a non-human or a human PH20, such as a PH20 has a sequence of amino acids that contains at least amino acids 36-464 of SEQ ID NO:1, or has a sequence of amino acids that has at least 85% sequence identity to a sequence of amino acids that contains at least amino acids 36-464 of SEQ ID NO:1 and retains hyaluronidase activity, or a PH20 that contains a sequence of amino acids that has at least 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO:1, and retains hyaluronidase activity. Exemplary of such PH20 polypeptides are those that have a sequence of amino acids that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1, or is a variant thereof that exhibits at least 85% sequence identity to a sequence of amino acids that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1 and retains hyaluronidase activity or has at least 86%, 87%, 88%, 89%, 90%, 91%, 92%, 93%, 94%, 95%, 96%, 97%, 98% or 99% sequence identity to a sequence of amino acids that contains that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1 and retains hyaluronidase activity. Included are hyaluronan-degrading enzymes that are C-terminal truncated PH20 enzymes that comprises have a sequence of amino set forth in any of SEQ ID NOS: 4-9.

In all methods provided herein, where fast-acting insulins are administered, alone or in a super-fast acting insulin composition, they can be monomeric, dimeric or hexameric. These include, a regular insulin, typically a human insulin, but they can be a pig insulin. The insulins include natural insulins isolated from animal sources, recombinantly produced insulins and synthetic insulins. Exemplary insulins include a regular insulin with an A chain having a sequence of amino acids set forth in SEQ ID NO:103 and a B chain having a sequence of amino acids set forth in SEQ ID NO:104 or an insulin with an A chain with a sequence of amino acids set forth as amino acid residue positions 88-108 of SEQ ID NO:123 and a B chain with a sequence of amino acids set forth as amino acid residue positions 25-54 of SEQ ID NO:123.

Also among the fast-acting insulins used, are the insulin analogs and any other insulins engineered to be similarly fast-acting or faster acting. Insulin analogs include those referred to as insulin aspart, insulin lispro or insulin glulisine. Exemplary of insulin analogs is the insulin analog selected from among an insulin having an A chain with a sequence of amino acids set forth in SEQ NOS:103 and a B chain having a sequence of amino acids set forth in any of SEQ NOS:147-149.

In the methods provided herein, the insulin composition can contain an amount that is between or about between 10 U/mL to 1000 U/mL, such as at or about 100 U/mL or between or about between 0.35 mg/mL to 3.5 mg/mL.

The compositions containing insulins can be super-fast acting insulin compositions, which are compositions that contain a fast-acting insulin, particularly an insulin analog, and a hyaluronan-degrading enzyme, such as any of those described above. The amount of hyaluronan degrading enzyme is an amount that renders the composition super-fast acting. The compositions can be formulated so that they are stable, particularly, so that the potency of the insulin remains at or above about 90% of its initial potency. Exemplary super-fast acting insulin compositions are formulated with appropriate salts, pH and preservatives and, if necessary, stabilizing agents so that they are stable for at least 3 days at a temperature from or from about 32° C. to 40° C., so that, for example the hyaluronan-degrading enzyme in the composition retains at least 50% of the initial hyaluronidase activity for at least 3 days at a temperature from or from about 32° C. to 40° C.; and the insulin in the composition retains: at least 90% potency or recovery of the initial level of insulin in the composition for at least 3 days at a temperature from or from about 32° C. to 40° C.; and/or at least 90% of the initial insulin purity for at least 3 days at a temperature from or from about 32° C. to 40° C. or; and/or less than 2% high molecular weight (HMWt) insulin species for at least 3 days at a temperature from or from about 32° C. to 40° C.

Exemplary of such super-fast acting insulin compositions are those that also have a pH of between or about between 6.5 to 7.5; and the composition contains: NaCl at a concentration between or about between 120 mM to 200 mM; an anti-microbial effective amount of a preservative or mixture of preservatives; and a stabilizing agent or agents. Stabilizing agents include hyaluronidase inhibitors and other compounds that prevent, inhibit or decrease degradation of the insulin and hyaluronidase degrading enzyme. Exemplary hyaluronidase inhibitor include, but are not limited to, a protein, a substrate of a hyaluronan-degrading enzyme, polysaccharides, fatty acid, lanostanoids, antibiotics, anti-nematodes, synthetic organic compounds and a plant-derived bioactive component, particularly inhibitors that do not form covalent complexes with either the hyaluronidase degrading enzyme or the insulin. Plant-derived bioactive components include, but are not limited to, an alkaloid, antioxidant, polyphenol, flavonoids, terpenoids and anti-inflammatory drugs. Other hyaluronidase inhibitors include, but are not limited to, a glycosaminoglycan (GAG), serum hyaluronidase inhibitor, Withania somnifera glycoprotein (WSG), heparin, heparin sulfate, dermatan sulfate, chitosans, β-(1,4)-galacto-oligosaccharides, sulphated verbascose, sulphated planteose, pectin, Poly(styrene-4-sulfonate), dextran sulfate, sodium alginate, Polysaccharide from Undaria pinnatifida, Mandelic acid condensation polymer, Eicosatrienoic acid, nervonic acid, oleanolic acid, aristolochic acid, ajmaline, reserpine, flavone, desmethoxycentauredine, quercetin, apigenin, kaempferol, silybin, luteolin, luteolin-7-glucoside, phloretin, apiin, hesperidin, sulphonated hesperidin, calycosin-7-0-β-D-glucopyranoside, Sodium flavone-7-sulphate, flavone 7-fluoro-4′-hydroxyflavone, 4′-chloro-4,6-dimethoxychalcone, sodium 5-hydroxyflavone 7-sulphate, myricetin, rutin, morin, glycyrrhizin, vitamin C, D-isoascorbic acid, D-saccharic 1-4 lactone, L-ascorbic acid-6-hexadecanoate (Vcpal), 6-O-acylated vitamin C, catechin, nordihydroguaiaretic acid, curcumin, N-propyl gallate, tannic acid, ellagic acid, gallic acid, phlorofucofuroeckol A, dieckol, 8,8′-bieckol, procyanidine, gossypol, celecoxib, nimesulide, dexamethasone, indomethcin, fenoprofen, phenylbutazone, oxyphenbutazone, salysylates, disodium cromoglycate, sodium aurothiomalate, transilist, traxanox, ivermectin, linocomycin and spectinomycin, sulfamethoxazole and trimerthoprim, neomycin sulphate, 3α-acetylpolyporenic acid A, (25S)-(+)-12α-hydroxy-3α-methylcarboxyacetate-24-methyllanosta-8,24(31)-diene-26-oic acid, lanostanoid, polyporenic acid c, PS53 (hydroquinone-sulfonic acid-formaldehyde polymer), polymer of poly (styrene-4-sulfonate), VERSA-TL 502, 1-tetradecane sulfonic acid, mandelic acid condensation polymer (SAMMA), 1,3-diacetylbenzimidazole-2-thione, N-monoacylated benzimidazol-2thione, N,N′-diacylated benzimidazol-2-thione, alkly-2-phenylindole derivate, 3-propanoylbenzoxazoke-2-thione, N-alkylated indole derivative, N-acylated indole derivate, benzothiazole derivative, N-substituted indole-2- and 3-carboxamide derivative, halogenated analogs (chloro and luroro) of N-substituted indole-2- and 3-carboxamide derivative, 2-(4-hydroxyphenyl)-3-phenylindole, indole carboxamides, indole acetamides, 3-benzolyl-1-methyl-4-phenyl-4-piperidinol, benzoyl phenyl benzoate derivative, 1-arginine derivative, guanidium HCL, L-NAME, HCN, linamarin, amygdalin, hederagenin, aescin, CIS-hinokiresinol and 1,3-di-P-hydroxyphenyl-4-penten-1-one. Also included are hyaluronidase inhibitors that are hyaluronidase substrates, such as a hyaluronan (HA) oligosaccharide, including, for example, a disaccharide or a tetrasaccharide. The HA oligosaccharide can contain a reacted reducing end so that it will not form complexes. The appropriate concentration of an inhibitor can be empirically determined. For example, the HA can be between or about between 1 mg/mL to 20 mg/mL.

Also provided are compositions containing a hyaluronan-degrading enzyme for use for minimizing the change in insulin absorption that occurs over a course of continuous subcutaneous insulin infusion (CSII) and uses of a hyaluronan-degrading enzyme composition for minimizing the change in insulin absorption that occurs over a course of continuous subcutaneous insulin infusion. The components and compositions for these uses are as described above for the methods for controlling blood glucose in a subject treated by continuous subcutaneous insulin infusion (CSII) therapy.

Also provided are uses of a composition and compositions for use as a leading edge in continuous subcutaneous insulin infusion (CSII) therapy for treatment of diabetes containing a hyaluronan-degrading enzyme that is formulated for direct administration in an amount that minimizes changes in insulin absorption that occurs over a course of continuous subcutaneous insulin infusion (CSII), whereby a leading edge therapeutic for insulin therapy is a composition that is administered prior to administration of an insulin composition by CSII. In the uses and compositions provided herein for leading edge therapy, the hyaluronan-degrading enzyme is in a therapeutically effective amount sufficient to catalyze the hydrolysis of hyaluronic acid to increase tissue permeability.

In particular examples of the compositions and uses for leading edge therapy, the hyaluronan-degrading enzyme is a hyaluronidase or a chondroitinase. For example, the hyaluronan-degrading enzyme is a hyaluronidase that is active at neutral pH. The hyaluronan-degrading enzyme includes one that lacks a glycosylphosphatidylinositol (GPI) anchor or is not membrane-associated when expressed from a cell. In some examples, the hyaluronan-degrading enzyme contains C-terminal truncations of one or more amino acid residues and lacks all or part of a GPI anchor.

In the uses and compositions for leading edge therapy provided herein, the composition can contain a hyaluronan-degrading enzyme that is a PH20 hyaluronidase. The PH20 can be non-human or a human PH20. The PH20 can have a sequence of amino acids that contains at least amino acids 36-464 of SEQ ID NO:1, or has a sequence of amino acids that has at least 85% sequence identity to a sequence of amino acids that contains at least amino acids 36-464 of SEQ ID NO:1 and retains hyaluronidase activity. For example, the PH20 in the composition can have at least 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO:1 and retains hyaluronidase activity. In some examples, the PH20 polypeptide has a sequence of amino acids that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1, or is a variant thereof that exhibits at least 85% sequence identity to a sequence of amino acids that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1 and retains hyaluronidase activity. For example, the PH20 has at least 86%, 87%, 88%, 89%, 90%, 91%, 92%, 93%, 94%, 95%, 96%, 97%, 98% or 99% sequence identity to a sequence of amino acids that contains that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1 and retains hyaluronidase activity. Exemplary of a hyaluronan-degrading enzyme in the compositions for leading edge therapy herein, the hyaluronan-degrading enzyme is a C-terminal truncated PH20 that has a sequence of amino set forth in any of SEQ ID NOS: 4-9, or a sequence of amino acids that exhibits at least 85% sequence identity to the sequence of amino acids set forth in any one of SEQ ID NOS:4-9. For example, the PH20 has a sequence of amino acids that exhibits at least 86%, 87%, 88%, 89%, 90%, 91%, 92%, 93%, 94%, 95%, 96%, 97%, 98% or 99% sequence identity to a sequence of amino acids set forth in any one of SEQ ID NOS:4-9. In particular examples, the PH20 has a sequence of amino acids set forth in any one of SEQ ID NOS:4-9.

In the uses and compositions for leading edge therapy provided herein, the hyaluronan-degrading enzyme in the composition is in an amount that is functionally equivalent to between or about between 1 Unit to 200 Units, 5 Units to 150 Units, 10 Units to 150 Units, 50 Units to 150 Units or 1 Unit to 50 Units. The hyaluronan-degrading enzyme in the composition can be in an amount that is between or about between 8 ng to 2 μg, 20 ng to 1.6 μg, 80 ng to 1.25 μg or 200 ng to 1 μg. In particular examples, the hyaluronan-degrading enzyme in the composition is in an amount from or from about 30 Units/mL to 3000 U/mL, 100 U/mL to 1000 U/mL, 300 U/mL to 2000 U/mL, 600 U/mL to 2000 U/mL or 600 U/mL to 1000 U/mL. For example, the hyaluronan-degrading enzyme in the composition is in an amount that is at least or about at least or 30 U/mL, 35 U/mL, 40 U/mL, 50 U/mL, 100 U/mL, 200 U/mL, 300 U/mL, 400 U/mL, 500 U/mL, 600 U/mL, 700 U/mL, 800 U/mL, 900 U/mL, 1000 U/ml, 2000 U/mL or 3000 U/mL.

In the uses and compositions provided herein for leading edge therapy, the insulin composition for use in continuous subcutaneous insulin infusion (CSII) therapy is a fast-acting insulin. The fast-acting insulin can be monomeric, dimeric or hexameric. The fast-acting insulin can be a fast-acting human insulin. In some examples, the fast-acting insulin is a regular insulin. For example, the regular insulin is a human insulin or pig insulin. The regular insulin can be an insulin with an A chain having a sequence of amino acids set forth in SEQ ID NO:103 and a B chain having a sequence of amino acids set forth in SEQ ID NO:104 or an insulin with an A chain with a sequence of amino acids set forth as amino acid residue positions 88-108 of SEQ ID NO:123 and a B chain with a sequence of amino acids set forth as amino acid residue positions 25-54 of SEQ ID NO:123. The fast-acting insulin can be a recombinant insulin, is synthesized or partially-synthesized or is isolated. In particular examples, the fast-acting insulin is an insulin analog. For example, the insulin analog can be an insulin having an A chain with a sequence of amino acids set forth in SEQ NOS:103 and a B chain having a sequence of amino acids set forth in any of SEQ NOS:147-149. In any of the compositions for use in leading edge therapy provided herein, the fast-acting insulin analog is insulin aspart, insulin lispro or insulin glulisine. The fast-acting insulin is formulated in the composition for continuous subcutaneous infusion in an amount that is from or from about 100 u/mL to 1000 U/mL or 500 U/mL to 1000 U/mL.

Also provided are compositions that contain insulin for bolus administration for use in ameliorating the decrease in total insulin action caused by a continuous subcutaneous insulin infusion of a super-fast acting insulin composition and uses of a bolus insulin for ameliorating the decrease in total insulin action caused by a continuous subcutaneous insulin infusion of a super-fast acting insulin composition. The components and compositions for these uses are as described above for the continuous subcutaneous insulin infusion (CSII) dosage regimens.

### BRIEF DESCRIPTION OF THE FIGURES

FIG. 1 depicts the serum immunoreactive insulin (IRI in pmol/L) concentration-versus-time for the 1^{st} clamp and 2^{nd} clamp study for both the insulin aspart (Novolog®) and Aspart-PH20 conditions. The figure shows that in the presence of PH20, aspart absorption is accelerated compared to aspart alone after both ½ day CSII (1^{st} clamp) and 2½ days CSII (2^{nd} clamp). The figure also shows that for both commercial aspart (Novolog®), insulin absorption was accelerated after 2½ days (2^{nd} clamp) relative to ½ day (1^{st} Clamp) CSII. This acceleration also was observed for the Aspart-PH20 conditions, but was reduced.

FIG. 2 depicts the glucodynamics of Aspart-PH20 compared to insulin aspart only (Novolog®) as measured by determining the infusion rate of glucose necessary to maintain euglycemia following the administration of bolus insulin.

FIG. 3 depicts total insulin action (cumulative glucose infused (Gtot)) as assayed by the euglycemic claim method. The Figure shows that total insulin action declined over the life of the infusion set, although to a greater degree for the insulin aspart-PH20 formulation.

FIG. 4 depicts the results as a cumulative time-action plot by normalizing for total insulin action. The Figure shows that percent (%) glucose infused accelerated from the 1^{st} Clamp to the 2^{nd} Clamp, and that addition of PH20 resulted in a faster time-action profile at both time points.

FIG. 5 depicts the pharmacokinetic profile of insulin infused by continuous subcutaneous administration with or without preadministration with rHuPH20 (leading edge). The results show that rHuPH20 preadministration accelerated insulin absorption at the beginning of infusion, and resulted in a decreased variability in insulin absorption as evidenced by no significant differences in early insulin exposure at the beginning of infusion set compared to the end of infusion set. In the absence of rHuPH20 preadministration, there was a variation insulin absorption as the infusion set aged.

FIG. 6 depicts the glucodynamics profile of insulin action as a function of time as evidenced by the rate of glucose infusion necessary to maintain euglycemia following a bolus insulin infusion. The results show that there was an accelerated onset of action of insulin (greater action and earlier onset of action) at the onset of infusion with pretreatment with rHuPH20 (leading edge, and shorter duration of action. In the absence of rHuPH20 preadministration, there was increased variation in insulin action as the infusion set aged.

### DETAILED DESCRIPTION

### Outline

A. DEFINITIONS
B. INSULIN THERAPY
   1. Insulin, Diabetes and Existing Fast-Acting Insulin Therapies
   2. Continuous Subcutaneous Infusion (CSII)
C. CONTINUOUS SUBCUTANEOUS INFUSION(CSII) METHODS OF INSULIN WITH A HYALURONAN-DEGRADING ENZYME
   1. Dosage Regimen Methods
      a. Leading Edge
      b. Method to Ameliorate Total Insulin Action
   2. Insulin pumps and other insulin delivery devices
      a. Open loop systems
      b. Closed loop systems
      c. Exemplary devices
D. INSULIN POLYPEPTIDES
   Fast-acting insulins
      a. Regular insulin
      b. Fast-acting analogs
         i. Insulin Lispro
         ii. Insulin Aspart
         iii. Insulin Glulisine
E. HYALURONAN DEGRADING ENZYMES
   1. Hyaluronidases
      a. Mammalian-type hyaluronidases PH20
      b. Bacterial hyaluronidases
      c. Hyaluronidases from leeches, other parasites and crustaceans
   2. Other hyaluronan degrading enzymes
   3. Truncated hyaluronan degrading enzymes or other soluble forms
      a. C-terminal Truncated Human PH20
      b. rHuPH20
   4. Glycosylation of hyaluronan degrading enzymes
   5. Modifications of hyaluronan degrading enzymes to improve their pharmacokinetic properties
F. SUPER FAST-ACTING INSULIN FORMULATIONS, AND STABLE FORMULATIONS THEREOF
   1. Stable Co-formulations
      a. NaCl and pH
      b. Hyaluronidase inhibitor
      c. Buffer
      d. Preservatives
      f. Stabilizer
         i. Surfactants
         ii. Other stabilizers
   2. Other Excipients or Agents
G. METHODS OF PRODUCING NUCLEIC ACIDS ENCODING AN INSULIN OR HYALURONAN DEGRADING ENZYME AND POLYPEPTIDES THEREOF
   1. Vectors and Cells
   2. Linker Moieties
   3. Expression
      a. Prokaryotic Cells
      b. Yeast Cells
      c. Insect Cells
      d. Mammalian Cells
      e. Plants
   4. Purification Techniques
H. THERAPEUTIC USES
   1. Diabetes Mellitus
      a. Type 1 diabetes
      b. Type 2 diabetes
      c. Gestational diabetes
   2. Insulin therapy for critically ill patients
I. COMBINATION THERAPIES
J. ARTICLES OF MANUFACTURE AND KITS
K. EXAMPLE

### A. DEFINITIONS

Unless defined otherwise, all technical and scientific terms used herein have the same meaning as is commonly understood by one of skill in the art to which the invention(s) belong. All patents, patent applications, published applications and publications, Genbank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, continuous subcutaneous insulin infusion therapy (CSII) refers to an insulin dosage regimen whereby insulin is administered by infusion at programmed rates over a course of several days from a small infuser or pump subcutaneously via an infusion set connected to the pump. Typically, CSII therapy continues for 2-4 days before the infusion set and pump reservoir must be replaced. The treatment combines continuous baseline insulin release (basal rate) and additional insulin bolus doses before meals and in response to high glycaemia values (i.e. correction bolus). CSII therapy generally uses a battery powered syringe driver, insulin pump or other similar device to deliver a fast-acting insulin, in particular an insulin analog, according to the dosage regimen. Generally, scheduling of continuous baseline insulin release is set by a physician for each patient. Bolus doses are determined based on prandial needs and glycemic responses. Hence, CSII therapy is patient specific. It is well within the level of a skilled physician and patient to determine the particular insulin dosage regimen for each patient depending on the needs of the patients and other patient-specific parameters such as weight, age, exercise, diet and clinical symptoms of the patient.

As used herein, an infusion set refers to a system attached to an insulin pump that directly delivers insulin from the reservoir in the pump to under the skin. Generally, an insulin infusion set contains one or more of a tubing system; a subcutaneous cannula, steel needle or other insertion device to insert the set under the skin; an adhesive mount to mount the insertion device to the site of administration, such as the abdominal wall; and/or a pump cartridge connector. The infusion set also can contain a quick-disconnect that leaves the insertion device and adhesive mount in place to permit the patient to conveniently remove the device, for example while performing activities such as showering or swimming.

As used herein, the basal rate of insulin refers to the body's insulin requirement without food. Generally, it is a pre-programmed or predetermined feature measured in units (U/H). Basal rates of insulin can change or vary depending on lifestyle variations, such as exercise, diet, or illness, and the patient's needs.

As used herein, the bolus rate or dose of insulin refers to additional insulin requirements to account for changes in insulin needs due to meals or to correct an elevated blood glucose level. Generally, bolus insulin is delivered by the user as needed and or is programmed to give a dose of insulin for meals, snacks and/or for correction of elevated blood glucose.

As used herein, a closed loop system is an integrated system for providing continuous glycemic control. Closed loop systems contain a mechanism for measuring blood glucose, a mechanism for delivering one or more compositions, including an insulin composition, and a mechanism for determining the amount of insulin needed to be delivered to achieve glycemic control. Typically, therefore, closed loop systems contain a glucose sensor, an insulin delivery device, such as an insulin pump, and a controller that receives information from the glucose sensor and provides commands to the insulin delivery device. The commands can be generated by software in the controller. The software typically includes an algorithm to determine the amount of insulin required to be delivered to achieve glycemic control, based upon the blood glucose levels detected by the glucose sensor or anticipated by the user.

An open loop system refers to devices similar to a closed-loop system, except that the devices do not automatically measure and respond to glucose levels. Generally, in an open-loop system an insulin pump or other similar device is programmed to infuse insulin continuously to deliver the basal rate of insulin, and where the patient is able, by means of a button on the pump or other manual means, to administer boluses of insulin at or near mealtime. The bolus dose administered is determined based on known or expected glucose levels, which can be manually monitored or can be monitored using a glucose monitor that displays real-time blood glucose results.

As used herein, “insulin” refers to a hormone, precursor or a synthetic or recombinant analog thereof that acts to increase glucose uptake and storage and/or decrease endogenous glucose production. An exemplary human insulin is translated as a 110 amino acid precursor polypeptide, preproinsulin (SEQ ID NO:101), containing a 24 amino acid signal peptide that directs the protein to the endoplasmic reticulum (ER) wherein the signal sequence is cleaved, resulting in proinsulin (SEQ ID NO:102). Proinsulin is processed further to release the 31 amino acid C— or connecting chain peptide (corresponding to amino acid residues 57 to 87 of the preproinsulin polypeptide set forth in SEQ ID NO:101, and to amino acid residues 33 to 63 of the proinsulin polypeptide set forth in SEQ ID NO:102). The resulting insulin contains a 21 amino acid A-chain (corresponding to amino acid residues 90 to 110 of the preproinsulin polypeptide set forth in SEQ ID NO:101, and to amino acid residues 66 to 86 of the proinsulin polypeptide set forth in SEQ ID NO:102) and a 30 amino acid B-chain (corresponding to amino acid residues 25 to 54 of the preproinsulin polypeptide set forth in SEQ ID NO:101, and to amino acid residues 1 to 30 of the proinsulin polypeptide set forth in SEQ ID NO:102) which are cross-linked by disulfide bonds. A properly cross-linked human insulin contains three disulfide bridges: one between position 7 of the A-chain and position 7 of the B-chain, a second between position 20 of the A-chain and position 19 of the B-chain, and a third between positions 6 and 11 of the A-chain. Reference to insulin includes preproinsulin, proinsulin and insulin polypeptides in single-chain or two-chain forms, truncated forms thereof that have activity, and includes allelic variants and species variants, variants encoded by splice variants, and other variants, such as insulin analogs, including polypeptides that have at least 40%, 45%, 50%, 55%, 60%, 65%, 70%, 75%, 80%, 85%, 90%, 95%, 96%, 97%, 98%, 99% or more sequence identity to the precursor polypeptide set forth in SEQ ID NO:101 or the mature form thereof. Exemplary insulin analogs include those having an A-chain set forth in SEQ ID NO:103 and a B-chain set forth in SEQ ID NOS:147-149, 152, and those containing an A-chain set forth in SEQ ID NOS:150, 156, 158, 160, 162 and 164 and/or a B chain set forth in SEQ ID NOS:151, 153-155, 157, 159, 161, 163 and 165.

Exemplary insulin polypeptides are those of mammalian, including human, origin. Exemplary amino acid sequences of insulin of human origin (A and B chain) are set forth in SEQ ID NOS: 101-104. Exemplary insulin analogs include those that have an A chain set forth in SEQ ID NO:103, and a B-chain set forth in SEQ ID NOS:147-149, 152, and those containing an A-chain set forth in SEQ ID NOS:150, 156, 158, 160, 162 and 164 and/or a B chain set forth in SEQ ID NOS:151, 153-155, 157, 159, 161, 163 and 165. Insulin polypeptides also include any of non-human origin including, but not limited to, any of the precursor insulin polypeptides set forth in SEQ ID NOS:105-146. Reference to an insulin includes monomeric and multimeric insulins, including hexameric insulins, as well as humanized insulins.

As used herein, “fast-acting insulin” refers to any insulin or fast-acting insulin composition for acute administration to a diabetic subject in response to an actual, perceived, or anticipated hyperglycemic condition in the subject arising at the time of, or within about four hours following, administration of the fast-acting insulin (such as a prandial hyperglycemic condition resulting or anticipated to result from, consumption of a meal), whereby the fast-acting insulin is able to prevent, control or ameliorate the acute hyperglycemic condition. Typically a fast-acting insulin is an insulin that exhibits peak insulin levels at or about not more than four hours following subcutaneous administration to a subject. Fast-acting insulins include recombinant insulins and isolated insulins (also referred to as “regular” insulins) such as the insulin sold as Humulin® R, porcine insulins and bovine insulins, as well as rapid acting insulin analogs (also termed fast-acting insulin analogs herein) designed to be rapid acting by virtue of amino acid changes. Exemplary regular insulin preparations include, but are not limited to, human regular insulins, such as those sold under the trademarks Humulin® R, Novolin® R and Velosulin®, Insulin Human, USP and Insulin Human Injection, USP, as well as acid formulations of insulin, such as, for example, Toronto Insulin, Old Insulin, and Clear Insulin, and regular pig insulins, such as Iletin II® (porcine insulin). Regular insulins typically have an onset of action of between 30 minutes to an hour, and a peak insulin level of 2-5 hours post administration.

As used herein, rapid acting insulin analogs (also called fast-acting insulin analogs) are insulins that have a rapid onset of action. Rapid insulins typically are insulin analogs that have been engineered, such as by the introduction of one or more amino acid substitutions, to be more rapid acting than regular insulins. Rapid acting insulin analogs typically have an onset of action of 10-30 minutes post injection, with peak insulin levels observed 30-90 minutes post injection. Exemplary rapid acting insulin analogs include, but are not limited to, for example, insulin lispro (e.g. Humalog® insulin), insulin aspart (e.g. NovoLog® insulin), and insulin glulisine (e.g. Apidra® insulin) the fast-acting insulin composition sold as VIAject® and VIAtab® (see, e.g., U.S. Pat. No. 7,279,457). Also included are any other insulins that have an onset of action of 30 minutes or less and a peak level before 90 minutes, typically 30-90 minutes, post injection.

As used herein, a human insulin refers to an insulin that is synthetic or recombinantly produced based upon the human polypeptide, including allelic variants and analogs thereof.

As used herein, fast-acting human insulins or human fast-acting insulin compositions include any human insulin or composition of a human insulin that is fast-acting, but excludes non-human insulins, such as regular pig insulin.

As used herein, the terms “basal-acting insulins,” or “basal insulins” refer to insulins administered to maintain a basal insulin level as part of an overall treatment regimen for treating a chronic condition such diabetes. Typically, a basal-acting insulin is formulated to maintain an approximately steady state insulin level by the controlled release of insulin when administered periodically (e.g. once or twice daily). Basal-acting insulins include crystalline insulins (e.g. NPH and Lente®, protamine insulin, surfen insulin), basal insulin analogs (insulin glargine, HOE 901, NovoSol Basal) and other chemical formulations of insulin (e.g. gum arabic, lecithin or oil suspensions) that retard the absorption rate of regular insulin. As used herein, the basal-acting insulins can include insulins that are typically understood as long-acting (typically reaching a relatively low peak concentration, while having a maximum duration of action over about 20-30 hours) or intermediate-acting (typically causing peak insulin concentrations at about 4-12 hours after administration).

As used herein, “glycemic” refers to blood sugar (glucose) levels.

As used herein, the terms “hyperglycemic condition” or “hyperglycemia” refer to an undesired elevation in blood glucose.

As used herein, the term “hypoglycemic condition” or “hypoglycemia” refers to an undesired drop in blood glucose.

As used herein, glycemic control or “controlling blood glucose levels” refers to the maintenance of blood glucose concentrations at a desired level, typically between 70-130 mg/dL or 90-110 mg/dL.

As used herein, glycosylated hemoglobin (HbAlc) test refers to a laboratory test that provides the percentage of a specific type of modified hemoglobin in the blood. The test ascertains the level of diabetic blood glucose control over the past three to four months.

As used herein, “insulin absorption” refers to the appearance of free and total insulin in the blood following injection. Methods of determining or measuring insulin absorption are well known to one of skill in the art, and include, but are not limited to, elimination or disappearance of radioactivity from the injection site (external gamma-counting) and/or appearance of plasma immunoreactive insulin (IRI) (see e.g. Fernqvist et al. (1988) Diabetes, 37:694-701; Bowsher (1999) Clinical Chemistry, 45:104-110). Methods of measuring plasma immunoreactive insulin includes conventional competitive radioimmunoassay (RIA) using a radiolabeled insulin tracer to trace insulin absorption and an anti-insulin antibody. Serum free insulin concentrations can be determined by RIA after precipitation with polyethylene glycol and serum total insulin concentrations can be determined with the same RIA procedures without polyethylene glycol precipitation.

As used herein, “insulin action” is a measure of insulin activity. It can be determined by measuring the glucose infusion rate needed to maintain isoglycemia during a euglycaemic clamp. It can be depicted as total glucose infused (g/kg) in a time interval.

As used herein, “total insulin action” is a measure of insulin action over the course of a euglycemic clamp experiment. It can be depicted as the cumulative glucose infused over the course of the experiment.

As used herein, “ultra-fast acting insulin response” refers to an insulin action response that exhibits a faster-in/faster-out (PK) profile such that there is an acceleration in insulin absorption and a shortened duration of action. As described herein, a “ultra-fast acting insulin response” is observed over time during the course of continuous infusion of insulin. Also, as described herein, a “ultra-fast acting insulin response” can be generated by leading edge therapy with a hyaluronan-degrading enzyme. For example, an ultra-fast acting insulin response can be generated within the first forty minutes to 1 hour following administration of a hyaluronan-degrading enzyme immediately before or immediately after infusion or injection of an insulin (e.g. within ±12 hours). Administration of a “super-fast acting insulin composition” also effects an “ultra-fast acting insulin response.”

As used herein, “leading edge therapy” with reference to continuous subcutaneous insulin infusion (CSII) refers to administration of a hyaluronan-degrading enzyme prior to administration of an insulin composition (e.g. a fast-acting insulin composition or a super-fast acting insulin composition) during an infusion set by continuous subcutaneous insulin infusion. The leading edge design primes the pump at the site of infusion, thereby increasing the rate of absorption of insulin at the beginning of infusion set life to thereby decrease the variability in insulin absorption that occurs as the infusion set ages. Reference to leading edge therapy generally only refers to a single interval or course of CSII therapy with an infusion set, which can be repeated during the course of treatment with subsequent infusion sets. At each interval, prior to infusion of insulin, a leading edge treatment with hyaluronan-degrading enzyme can be administered. The leading edge administration is generally given within 12 (twelve hours) prior to administration of insulin, and generally within 2 hours of administration or less.

As used herein, “super fast-acting insulin composition” refers to an insulin composition containing a fast-acting insulin, typically a fast-acting insulin analog, and a hyaluronan degrading enzyme (such as a soluble hyaluronidase, including but not limited to, rHuPH20 preparations), such that the insulin composition, over the first forty minutes following parenteral administration to a subject, provides a cumulative systemic insulin exposure in the subject that is greater than the cumulative systemic insulin exposure provided to the subject over the same period after administering the same dosage of the same fast-acting insulin, by the same route, in the absence of the hyaluronan degrading enzyme. The super fast-acting insulin composition as described herein optionally can include a basal-acting insulin.

As used herein, dosing regime refers to the amount of insulin administered and the frequency of administration. The dosing regime is a function of the disease or condition to be treated, and thus can vary.

As used herein, a hyaluronan degrading enzyme refers to an enzyme that catalyzes the cleavage of a hyaluronan polymer (also referred to as hyaluronic acid or HA) into smaller molecular weight fragments. Exemplary of hyaluronan degrading enzymes are hyaluronidases, and particular chondroitinases and lyases that have the ability to depolymerize hyaluronan. Exemplary chondroitinases that are hyaluronan degrading enzymes include, but are not limited to, chondroitin ABC lyase (also known as chondroitinase ABC), chondroitin AC lyase (also known as chondroitin sulfate lyase or chondroitin sulfate eliminase) and chondroitin C lyase. Chondroitin ABC lyase comprises two enzymes, chondroitin-sulfate-ABC endolyase (EC 4.2.2.20) and chondroitin-sulfate-ABC exolyase (EC 4.2.2.21). An exemplary chondroitin-sulfate-ABC endolyases and chondroitin-sulfate-ABC exolyases include, but are not limited to, those from Proteus vulgaris and Flavobacterium heparinum (the Proteus vulgaris chondroitin-sulfate-ABC endolyase is set forth in SEQ ID NO:98; Sato et al. (1994) Appl. Microbiol. Biotechnol. 41(1):39-46). Exemplary chondroitinase AC enzymes from the bacteria include, but are not limited to, those from Flavobacterium heparinum, set forth in SEQ ID NO:99, Victivallis vadensis, set forth in SEQ ID NO:100 and Arthrobacter aurescens (Tkalec et al. (2000) Applied and Environmental Microbiology 66(1):29-35; Ernst et al. (1995) Critical Reviews in Biochemistry and Molecular Biology 30(5):387-444). Exemplary chondroitinase C enzymes from the bacteria include, but are not limited to, those from Streptococcus and Flavobacterium (Hibi et al. (1989) FEMS-Microbiol-Lett. 48(2):121-4; Michelacci et al. (1976) J. Biol. Chem. 251:1154-8; Tsuda et al. (1999) Eur. I Biochem. 262:127-133).

As used herein, hyaluronidase refers to a class of hyaluronan degrading enzymes. Hyaluronidases include bacterial hyaluronidases (EC 4.2.2.1 or EC 4.2.99.1), hyaluronidases from leeches, other parasites, and crustaceans (EC 3.2.1.36), and mammalian-type hyaluronidases (EC 3.2.1.35). Hyaluronidases include any of non-human origin including, but not limited to, murine, canine, feline, leporine, avian, bovine, ovine, porcine, equine, piscine, ranine, bacterial, and any from leeches, other parasites, and crustaceans. Exemplary non-human hyaluronidases include, hyaluronidases from cows (SEQ ID NOS:10, 11, 64 and BH55 (U.S. Pat. Nos. 5,747,027 and 5,827,721), yellow jacket wasp (SEQ ID NOS:12 and 13), honey bee (SEQ ID NO:14), white-face hornet (SEQ ID NO:15), paper wasp (SEQ ID NO:16), mouse (SEQ ID NOS:17-19, 32), pig (SEQ ID NOS:20-21), rat (SEQ ID NOS:22-24, 31), rabbit (SEQ ID NO:25), sheep (SEQ ID NOS:26, 27, 63 and 65), orangutan (SEQ ID NO:28), cynomolgus monkey (SEQ ID NO:29), guinea pig (SEQ ID NO:30), chimpanzee (SEQ ID NO:185), rhesus monkey (SEQ ID NO:186), Arthrobacter sp. (strain FB24) (SEQ ID NO:67), Bdellovibrio bacteriovorus (SEQ ID NO:68), Propionibacterium acnes (SEQ ID NO:69), Streptococcus agalactiae (SEQ ID NO:70); 18RS21 (SEQ ID NO:71); serotype 1a (SEQ ID NO:72); serotype III (SEQ ID NO:73), Staphylococcus aureus (strain COL) (SEQ ID NO:74); strain MRSA252 (SEQ ID NOS:75 and 76); strain MSSA476 (SEQ ID NO:77); strain NCTC 8325 (SEQ ID NO:78); strain bovine RF122 (SEQ ID NOS:79 and 80); strain USA300 (SEQ ID NO:81), Streptococcus pneumoniae (SEQ ID NO:82); strain ATCC BAA-255/R6 (SEQ ID NO:83); serotype 2, strain D39/NCTC 7466 (SEQ ID NO:84), Streptococcus pyogenes (serotype M1) (SEQ ID NO:85); serotype M2, strain MGAS10270 (SEQ ID NO:86); serotype M4, strain MGAS10750 (SEQ ID NO:87); serotype M6 (SEQ ID NO:88); serotype M12, strain MGAS2096 (SEQ ID NOS:89 and 90); serotype M12, strain MGAS9429 (SEQ ID NO:91); serotype M28 (SEQ ID NO:92); Streptococcus suis (SEQ ID NOS:93-95); Vibrio fischeri (strain ATCC 700601/ES114 (SEQ ID NO:96)), and the Streptomyces hyaluronolyticus hyaluronidase enzyme, which is specific for hyaluronic acid and does not cleave chondroitin or chondroitin sulfate (Ohya, T. and Kaneko, Y. (1970) Biochim. Biophys. Acta 198:607). Hyaluronidases also include those of human origin. Exemplary human hyaluronidases include HYAL1 (SEQ ID NO:36), HYAL2 (SEQ ID NO:37), HYAL3 (SEQ ID NO:38), HYAL4 (SEQ ID NO:39), and PH20 (SEQ ID NO:1). Also included amongst hyaluronidases are soluble hyaluronidases, including, ovine and bovine PH20, soluble human PH20 and soluble rHuPH20. Examples of commercially available bovine or ovine soluble hyaluronidases Vitrase® (ovine hyaluronidase) and Amphadase® (bovine hyaluronidase).

Reference to hyaluronan degrading enzymes includes precursor hyaluronan degrading enzyme polypeptides and mature hyaluronan degrading enzyme polypeptides (such as those in which a signal sequence has been removed), truncated forms thereof that have activity, and includes allelic variants and species variants, variants encoded by splice variants, and other variants, including polypeptides that have at least 40%, 45%, 50%, 55%, 60%, 65%, 70%, 75%, 80%, 85%, 90%, 95%, 96%, 97%, 98%, 99% or more sequence identity to the precursor polypeptides set forth in SEQ ID NOS: 1 and 10-48, 63-65, 67-100, or the mature form thereof. For example, reference to a hyaluronan-degrading enzyme (e.g. PH20) includes the mature human PH20 set forth in SEQ ID NO:2 and truncated forms thereof that have activity, and includes allelic variants and species variants, variants encoded by splice variants and other variants including polypeptides that have at least 40%, 45%, 50%, 55%, 60%, 65%, 70%, 75%, 80%, 85%, 90%, 95%, 96%, 97%, 98%, 99% or more sequence identity to SEQ ID NO:2. For example, reference to hyaluronan degrading enzyme also includes the human PH20 precursor polypeptide variants set forth in SEQ ID NOS:50-51. Hyaluronan degrading enzymes also include those that contain chemical or posttranslational modifications and those that do not contain chemical or posttranslational modifications. Such modifications include, but are not limited to, pegylation, albumination, glycosylation, farnesylation, carboxylation, hydroxylation, phosphorylation, and other polypeptide modifications known in the art.

As used herein, PH20 refers to a type of hyaluronidase that occurs in sperm and is neutral-active. PH-20 occurs on the sperm surface, and in the lysosome-derived acrosome, where it is bound to the inner acrosomal membrane. PH20 includes those of any origin including, but not limited to, human, chimpanzee, Cynomolgus monkey, Rhesus monkey, murine, bovine, ovine, guinea pig, rabbit and rat origin. Exemplary PH20 proteins include, but are not limited to, human (precursor polypeptide set forth in SEQ ID NO:1, mature polypeptide set forth in SEQ ID NO: 2), bovine (SEQ ID NOS: 11 and 64), rabbit (SEQ ID NO: 25), ovine PH20 (SEQ ID NOS: 27, 63 and 65), cynomolgus monkey (SEQ ID NO: 29), guinea pig (SEQ ID NO: 30), rat (SEQ ID NO: 31), mouse (SEQ ID NO: 32), chimpanzee (SEQ ID NO: 185) and rhesus monkey (SEQ ID NO:186) PH20 polypeptides. Reference to PH20 includes precursor PH20 polypeptides and mature PH20 polypeptides (such as those in which a signal sequence has been removed), truncated forms thereof that have activity, and includes allelic variants and species variants, variants encoded by splice variants, and other variants, including polypeptides that have at least 40%, 45%, 50%, 55%, 65%, 70%, 75%, 80%, 85%, 90%, 95%, 96%, 97%, 98%, 99% or more sequence identity to the precursor polypeptides set forth in SEQ ID NO:1, 11, 25, 27, 29-32, 63-65, 185 or 186, or the mature forms thereof. PH20 polypeptides also include those that contain chemical or posttranslational modifications and those that do not contain chemical or posttranslational modifications. Such modifications include, but are not limited to, pegylation, albumination, glycosylation, farnesylation, carboxylation, hydroxylation, phosphorylation, and other polypeptide modifications known in the art. Examples of commercially available bovine or ovine soluble hyaluronidases are Vitrase® hyaluronidase (ovine hyaluronidase) and Amphadase® hyaluronidase (bovine hyaluronidase).

As used herein, a soluble hyaluronidase refers to a polypeptide that is secreted from cells and is not membrane-anchored or associated, and hence can be characterized by its solubility under physiologic conditions. Soluble hyaluronidases can be distinguished, for example, by its partitioning into the aqueous phase of a Triton X-114 solution warmed to 37° C. (Bordier et al., (1981) J. Biol. Chem., 256:1604-7). Membrane-anchored, such as lipid anchored hyaluronidases, will partition into the detergent rich phase, but will partition into the detergent-poor or aqueous phase following treatment with Phospholipase-C. Included among soluble hyaluronidases are membrane anchored hyaluronidases in which one or more regions associated with anchoring of the hyaluronidase to the membrane has been removed or modified, where the soluble form retains hyaluronidase activity. Soluble hyaluronidases include recombinant soluble hyaluronidases and those contained in or purified from natural sources, such as, for example, testes extracts from sheep or cows. Exemplary of such soluble hyaluronidases are soluble human PH20. Other soluble hyaluronidases include ovine (SEQ ID NOS:27, 63, 65) and bovine (SEQ ID NOS:11, 64) PH20.

As used herein, soluble human PH20 or sHuPH20 include mature polypeptides lacking all or a portion of the glycosylphosphatidylinositol (GPI) attachment site at the C-terminus such that upon expression, the polypeptides are not associated with the membrane of a host cell in which they are produced so that they are secreted and, thus, soluble in the cell culture medium. Hence, soluble human PH20 includes C-terminal truncated human PH20 polypeptides. Exemplary soluble or C-terminal truncated PH20 polypeptides include mature polypeptides having an amino acids sequence set forth in any one of SEQ ID NOS: 4-9, 47-48, 234-254, and 267-273, or a polypeptide that exhibits at least 70%, 75%, 80%, 85%, 86%, 87%, 88%, 89%, 90%, 91%, 92%, 93%, 94%, 95%, 96%, 97%, 98%, 99% or more sequence identity to any of SEQ ID NOS: 4-9, 47-48, 234-254, and 267-273. Exemplary sHuPH20 polypeptides include mature polypeptides having an amino acid sequence set forth in any one of SEQ ID NOS:4-9 and 47-48. The precursor polypeptides for such exemplary sHuPH20 polypeptides include a signal sequence. Exemplary of the precursors are those set forth in SEQ ID NOS:3 and 40-46, each of which contains a 35 amino acid signal sequence at amino acid positions 1-35. Soluble HuPH20 polypeptides also include those degraded during or after the production and purification methods described herein.

As used herein, a recombinant human PH20 referred to as rHuPH20 refers to a secreted soluble form of human PH20 that is recombinantly expressed in Chinese Hamster Ovary (CHO) cells. Soluble rHuPH20 is the product produced by nucleic acid that encodes a signal sequence, such as the native signal sequence, and includes nucleic acid that encodes amino acids 36-482 and for which an exemplary sequence, including the nucleic acid encoding the native signal sequence is set forth in SEQ ID NO:49. Also included are DNA molecules that are allelic variants thereof and other soluble variants. The nucleic acid encoding soluble rHuPH20 is expressed in CHO cells, which secrete the mature polypeptide. As produced in the culture medium, there is heterogeneity at the C-terminus so that the product includes a mixture of species that can include any one or more of SEQ ID NOS. 4-9 in various abundance. Corresponding allelic variants and other variants also are included, including those corresponding to the precursor human PH20 polypeptides set forth in SEQ ID NOS:50-51. Other variants can have 60%, 70%, 80%, 85%, 90%, 91%, 92%, 93%, 94%, 95%, 96%, 97%, 98%, 99% or more sequence identity with any of SEQ ID NOS:4-9 and 47-48 as long they retain a hyaluronidase activity and are soluble.

As used herein, a formulation refers to a composition containing at least one active pharmaceutical agent and one or more excipients.

As used herein, a co-formulation refers to a composition containing two or more active pharmaceutical agents and one or more excipients. For example, a co-formulation of a fast-acting insulin and a hyaluronan degrading enzyme contains a fast-acting insulin, a hyaluronan degrading enzyme, and one or more excipients.

As used herein, a composition is said to be stable under defined conditions if the active ingredients therein retains at least a requisite level of activity and/or purity and/or potency or recovery compared to the initial activity and/or purity and/or potency or recovery. For purposes herein, a composition is stable if it retains at least 50% of the hyaluronan-degrading enzyme activity and/or if it retains at least 90% of insulin potency or recovery and/or at least 90% of the insulin purity.

As used herein, a stable co-formulation, which contains at least two active ingredients, is stable if each active ingredient retains at least the requisite level of activity and/or purity and/or potency or recovery compared to the initial activity and/or purity and/or potency or recovery. For purposes herein, a coformulation is stable if it retains at least 50% of the hyaluronan-degrading enzyme activity and if it retains at least 90% of insulin potency or recovery and/or at least 90% of the insulin purity.

As used herein, defined conditions refer to conditions of storage and/or use.

As used herein, defined conditions for storage or use under which stability is measured includes temperature conditions, time of storage conditions and/or use conditions. For example, defined temperature conditions include low or refrigerated temperatures of 2° C. to 8° C., ambient temperatures of 20° C. to 30° C. or elevated temperatures of 32° C. to 40° C. In another example, defined time conditions refers to the length of storage under varied temperature conditions, such as storage for days (at least 3 days, 4 days, 5 days, 6 days or 7 days), weeks (at least one week, at least two weeks, at least three weeks or at least for weeks) or months (at least 1 months, 2 months, 3 months, 4 months, 5 months, 6 months, 12 months, 18 months, 24 months or more). In a further example, defined use conditions refers to conditions that disturb or alter the composition mixture, such as conditions of agitation.

As used herein, “storage” means that a formulation is not immediately administered to a subject once prepared, but is kept for a period of time under particular conditions (e.g. particular temperature; time, liquid or lyophilized form) prior to use. For example, a liquid formulation can be kept for days, weeks, months or years, prior to administration to a subject under varied temperatures such as refrigerated (0° to 10° C., such as 2° to 8° C.), room temperature (e.g. temperature up to 32° C., such as 18° C. to about or at 32° C.), or elevated temperature (e.g., 30° C. to 42° C., such as 32° C. to 37° C. or 35° C. to 37° C.).

As used herein, “use” with reference to a condition associated with stability refers to the act of employing the formulation for a specific purpose. Particular applications can influence the activity or properties of a protein or agent. For example, certain applications can require that the formulation is subjected to certain temperatures for certain time periods, is subjected to fluctuations in temperature and or is subjected to agitation, shaking, stirring or other similar motion that can affect the stability (e.g. activity and/or solubility) of the active agents. Exemplary of a condition is continuous infusion methods, whereby active agents are continuously infused to a subject from a user-associated pump or infuser over a course of several days. Such a condition can be associated with agitation and fluctuations in temperature.

As used herein, a single dosage formulation refers to a formulation or co-formulation for direct administration. Generally, a single dosage formulation is a formulation that contains a single dose of therapeutic agent for direct administration. Single dosage formulations generally do not contain any preservatives.

As used herein, a multi-dose formulation refers to a formulation that contains multiple doses of a therapeutic agent and that can be directly administered to provide several single doses of the therapeutic agent. The doses can be administered over the course of minutes, hours, weeks, days or months. Multidose formulations can allow dose adjustment, dose-pooling and/or dose-splitting. Because multi-dose formulations are used over time, they generally contain one or more preservatives to prevent microbial growth. Multi-dose formulations can be formulated for injection or infusion (e.g. continuous infusion).

As used herein, a “stable multiple dose injection co-formulation” refers to a stable co-formulation that is stable for at least 6 months at a temperature from or from about 2° C. to 8° C. and/or for at least 14 days at a temperature from or from about 20° C. to 30° C., such that the requisite level of activity and/or purity and/or potency or recovery is retained over the defined time and temperature compared to the initial activity and/or purity and/or potency or recovery. For example, a stable multiple dose injection formulation retains at least 50% of the hyaluronan-degrading enzyme activity and at least 90% of insulin potency or recovery and/or at least 90% of the insulin purity for at least 6 months at a temperature from or from about 2° C. to 8° C. and/or for at least 14 days at a temperature from or from about 20° C. to 30° C.

As used herein, a “stable continuous insulin infusion formulation” refers to a stable co-formulation that is stable for at least 3 days at a temperature from or from about 32° C. to 40° C., such that the requisite level of activity and/or purity and/or potency or recovery is retained over the defined time and temperature compared to the initial activity and/or purity and/or potency or recovery. For example, a stable continuous insulin infusion formulation retains at least 50% of the hyaluronan-degrading enzyme activity and at least 90% of insulin potency or recovery and/or at least 90% of the insulin purity for at least 3 days at a temperature from or from about 32° C. to 40° C.

As used herein, a stabilizing agent refers to compound added to the formulation to protect either the hyaluronan degrading enzyme or insulin or both from degradation, such as under the conditions of salt, pH and temperature at which the co-formulations herein are stored or used. Thus, included are agents that prevent proteins from degradation from other components in the compositions. Hence, they are protein stabilizing agents. Exemplary of such agents are amino acids, amino acid derivatives, amines, sugars, polyols, salts and buffers, surfactants, inhibitors or substrates and other agents as described herein.

As used herein, an antimicrobial effectiveness test demonstrates the effectiveness of the preservative system in a product. A product is inoculated with a controlled quantity of specific organisms. The test then compares the level of microorganisms found on a control sample versus the test sample over a period of 28 days. Parameters for performing an antimicrobial effectiveness test are known to one of skill in the art as described herein.

As used herein, an anti-microbially or anti-microbial effective amount of a preservative refers to an amount of the preservative that kills or inhibits the propagation of microbial organisms in a sample that may be introduced from storage or use. For example, for multiple-dose containers, an anti-microbially effective amount of a preservative inhibits the growth of microorganisms that may be introduced from repeatedly withdrawing individual doses. USP and EP (EPA and EPB) have anti-microbial requirements that determine preservative effectiveness, and that vary in stringency. For example, an anti-microbial effective amount of a preservative is an amount such that at least a 1.0 log₁₀ unit reduction in bacterial organisms occurs at 7 days following inoculation in an antimicrobial preservative effectiveness test (APET). In a particular example, an anti-microbial effective amount of a preservative is an amount such that at least a 1.0 log₁₀ unit reduction in bacterial organisms occurs at 7 days following inoculation, at least a 3.0 log₁₀ unit reduction of bacterial organisms occurs at 14 days following inoculation at least no further increase in bacterial organisms occurs after 28 days following inoculation; and at least no increase in fungal organisms occurs after 7 days following inoculation. In a further example, an anti-microbial effective amount of a preservative is an amount such that at least a 1.0 log₁₀ unit reduction of bacterial organisms occurs at 24 hours following inoculation, at least a 3.0 log₁₀ unit reduction of bacterial organisms occurs at 7 days following inoculation, no further increase in bacterial organisms occurs after 28 days following inoculation, at least a 1.0 log₁₀ unit reduction of fungal organisms occurs at 14 days following inoculation, and at least no further increase in fungal organisms occurs after 28 days following inoculation. In an additional example, an anti-microbial effective amount of a preservative is an amount such that at least a 2.0 log₁₀ unit reduction of bacterial organisms at 6 hours following inoculation, at least a 3.0 log₁₀ unit reduction of bacterial organisms occurs at 24 hours following inoculation, no recovery of bacterial organisms occurs after 28 days following inoculation of the composition with the microbial inoculum, at least a 2.0 log₁₀ unit reduction of fungal organisms occurs at 7 days following inoculation, and at least no further increase in fungal organisms occurs after 28 days following inoculation.

As used herein, the “excipient” refers to a compound in a formulation of an active agent that does not provide the biological effect of the active agent when administered in the absence of the active agent. Exemplary excipients include, but are not limited to, salts, buffers, stabilizers, tonicity modifiers, metals, polymers, surfactants, preservatives, amino acids and sugars.

As used herein, a “buffer” refers to a substance, generally a solution, that can keep its pH constant, despite the addition of strong acids or strong bases and external influences of temperature, pressure, volume or redox potential. Buffer prevents change in the concentration of another chemical substance, e.g. proton donor and acceptor systems that prevent marked changes in hydrogen ion concentration (pH). The pH values of all buffers are temperature and concentration dependent. The choice of buffer to maintain a pH value or range can be empirically determined by one of skill in the art based on the known buffering capacity of known buffers. Exemplary buffers include but are not limited to, bicarbonate buffer, cacodylate buffer, phosphate buffer or Tris buffer. For example, Tris buffer (tromethamine) is an amine based buffer that has a pKa of 8.06 and has an effective pH range between 7.9 and 9.2. For Tris buffers, pH increases about 0.03 unit per ° C. temperature decrease, and decreases 0.03 to 0.05 unit per ten-fold dilution.

As used herein, activity refers to a functional activity or activities of a polypeptide or portion thereof associated with a full-length (complete) protein. Functional activities include, but are not limited to, catalytic or enzymatic activity, antigenicity (ability to bind or compete with a polypeptide for binding to an anti-polypeptide antibody), immunogenicity, ability to form multimers, and the ability to specifically bind to a receptor or ligand for the polypeptide.

As used herein, hyaluronidase activity refers to the ability to enzymatically catalyze the cleavage of hyaluronic acid. The United States Pharmacopeia (USP) XXII assay for hyaluronidase determines hyaluronidase activity indirectly by measuring the amount of higher molecular weight hyaluronic acid, or hyaluronan, (HA) substrate remaining after the enzyme is allowed to react with the HA for 30 min at 37° C. (USP XXII-NF XVII (1990) 644-645 United States Pharmacopeia Convention, Inc, Rockville, Md.). A Reference Standard solution can be used in an assay to ascertain the relative activity, in units, of any hyaluronidase. In vitro assays to determine the hyaluronidase activity of hyaluronidases, such as soluble rHuPH20, are known in the art and described herein. Exemplary assays include the microturbidity assay described below (see e.g. Example 8) that measures cleavage of hyaluronic acid by hyaluronidase indirectly by detecting the insoluble precipitate formed when the uncleaved hyaluronic acid binds with serum albumin. Reference Standards can be used, for example, to generate a standard curve to determine the activity in Units of the hyaluronidase being tested.

As used herein, “functionally equivalent amount” or grammatical variations thereof, with reference to a hyaluronan degrading enzyme, refers to the amount of hyaluronan degrading enzyme that achieves the same effect as an amount (such as a known number of Units of hyaluronidase activity) of a reference enzyme, such as a hyaluronidase. For example, the activity of any hyaluronan degrading enzyme can be compared to the activity of rHuPH20 to determine the functionally equivalent amount of a hyaluronan degrading enzyme that would achieve the same effect as a known amount of rHuPH20. For example, the ability of a hyaluronan degrading enzyme to act as a spreading or diffusing agent can be assessed by injecting it into the lateral skin of mice with trypan blue (see e.g. U.S. Pat. Publication No. 20050260186), and the amount of hyaluronan degrading enzyme required to achieve the same amount of diffusion as, for example, 100 units of a Hyaluronidase Reference Standard, can be determined. The amount of hyaluronan degrading enzyme required is, therefore, functionally equivalent to 100 units. In another example, the ability of a hyaluronan degrading enzyme to increase the level and rate of absorption of a co-administered insulin can be assessed in human subjects, such as described below in Example 1, and the amount of hyaluronan degrading enzyme required to achieve the same increase in the level and rate of absorption of insulin as, for example, the administered quantity of rHuPH20, can be determined (such as by assessing the maximum insulin concentration in the blood (Cₘₐₓ,) the time required to achieve maximum insulin concentration in the blood (tₘₐₓ) and the cumulative systemic insulin exposure over e given period of time (AUC).

As used herein, nucleic acids include DNA, RNA and analogs thereof, including peptide nucleic acids (PNA) and mixtures thereof. Nucleic acids can be single or double-stranded. When referring to probes or primers, which are optionally labeled, such as with a detectable label, such as a fluorescent or radiolabel, single-stranded molecules are contemplated. Such molecules are typically of a length such that their target is statistically unique or of low copy number (typically less than 5, generally less than 3) for probing or priming a library. Generally a probe or primer contains at least 14, 16 or 30 contiguous nucleotides of sequence complementary to or identical to a gene of interest. Probes and primers can be 10, 20, 30, 50, 100 or more nucleic acids long.

As used herein, a peptide refers to a polypeptide that is greater than or equal to two amino acids in length, and less than or equal to 40 amino acids in length.

As used herein, the amino acids that occur in the various sequences of amino acids provided herein are identified according to their known, three-letter or one-letter abbreviations (Table 1). The nucleotides that occur in the various nucleic acid fragments are designated with the standard single-letter designations used routinely in the art.

As used herein, an “amino acid” is an organic compound containing an amino group and a carboxylic acid group. A polypeptide contains two or more amino acids. For purposes herein, amino acids include the twenty naturally-occurring amino acids, non-natural amino acids and amino acid analogs (i.e., amino acids wherein the α-carbon has a side chain).

As used herein, “amino acid residue” refers to an amino acid formed upon chemical digestion (hydrolysis) of a polypeptide at its peptide linkages. The amino acid residues described herein are presumed to be in the “L” isomeric form. Residues in the “D” isomeric form, which are so designated, can be substituted for any L-amino acid residue as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243: 3557-3559 (1968), and adopted 37 C.F.R. §§1.821-1.822, abbreviations for amino acid residues are shown in Table 1:

TABLE 1
Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | proline |
| K | Lys | Lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | asparagine |
| B | Asx | Asn and/or Asp |
| C | Cys | Cysteine |
| X | Xaa | Unknown or other |

All amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence (Table 1) and modified and unusual amino acids, such as those referred to in 37 C.F.R. §§1.821-1.822, and incorporated herein by reference. Furthermore, a dash at the beginning or end of an amino acid residue sequence indicates a peptide bond to a further sequence of one or more amino acid residues, to an amino-terminal group such as NH, or to a carboxyl-terminal group such as COOH.

As used herein, “naturally occurring amino acids” refer to the 20 L-amino acids that occur in polypeptides.

As used herein, “non-natural amino acid” refers to an organic compound that has a structure similar to a natural amino acid but has been modified structurally to mimic the structure and reactivity of a natural amino acid. Non-naturally occurring amino acids thus include, for example, amino acids or analogs of amino acids other than the 20 naturally-occurring amino acids and include, but are not limited to, the D-isostereomers of amino acids. Exemplary non-natural amino acids are described herein and are known to those of skill in the art.

As used herein, a DNA construct is a single- or double-stranded, linear or circular DNA molecule that contains segments of DNA combined and juxtaposed in a manner not found in nature. DNA constructs exist as a result of human manipulation, and include clones and other copies of manipulated molecules.

As used herein, a DNA segment is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, the term polynucleotide means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term nucleotides is used for single- and double-stranded molecules where the context permits. When the term is applied to double-stranded molecules it is used to denote overall length and will be understood to be equivalent to the term base pairs. It will be recognized by those skilled in the art that the two strands of a double-stranded polynucleotide can differ slightly in length and that the ends thereof can be staggered; thus all nucleotides within a double-stranded polynucleotide molecule may not be paired. Such unpaired ends will, in general, not exceed 20 nucleotides in length.

As used herein, “similarity” between two proteins or nucleic acids refers to the relatedness between the sequence of amino acids of the proteins or the nucleotide sequences of the nucleic acids. Similarity can be based on the degree of identity and/or homology of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences.

As used herein, “identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions). “Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exists a number of methods to measure identity between two polynucleotide or polypeptides, the term “identity” is well known to skilled artisans (Carrillo, H. & Lipton, D., SIAM J Applied Math 48:1073 (1988)).

As used herein, homologous (with respect to nucleic acid and/or amino acid sequences) means about greater than or equal to 25% sequence homology, typically greater than or equal to 25%, 40%, 50%, 60%, 70%, 80%, 85%, 90% or 95% sequence homology; the precise percentage can be specified if necessary. For purposes herein the terms “homology” and “identity” are often used interchangeably, unless otherwise indicated. In general, for determination of the percentage homology or identity, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48:1073). By sequence homology, the number of conserved amino acids is determined by standard alignment algorithms programs, and can be used with default gap penalties established by each supplier. Substantially homologous nucleic acid molecules would hybridize typically at moderate stringency or at high stringency all along the length of the nucleic acid of interest. Also contemplated are nucleic acid molecules that contain degenerate codons in place of codons in the hybridizing nucleic acid molecule.

Whether any two molecules have nucleotide sequences or amino acid sequences that are at least 60%, 70%, 80%, 85%, 90%, 95%, 96%, 97%, 98% or 99% “identical” or “homologous” can be determined using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA (Altschul, S. F., et al., J Molec Biol 215:403 (1990)); Guide to Huge Computers, Martin J. Bishop, ed., Academic Press, San Diego, 1994, and Carrillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman (1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Therefore, as used herein, the term “identity” or “homology” represents a comparison between a test and a reference polypeptide or polynucleotide. As used herein, the term at least “90% identical to” refers to percent identities from 90 to 100% relative to the reference nucleic acid or amino acid sequence of the polypeptide. Identity at a level of 90% or more is indicative of the fact that, assuming for exemplification purposes a test and reference polypeptide length of 100 amino acids are compared. No more than 10% (i.e., 10 out of 100) of the amino acids in the test polypeptide differs from that of the reference polypeptide. Similar comparisons can be made between test and reference polynucleotides. Such differences can be represented as point mutations randomly distributed over the entire length of a polypeptide or they can be clustered in one or more locations of varying length up to the maximum allowable, e.g. 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often by manual alignment without relying on software.

As used herein, an aligned sequence refers to the use of homology (similarity and/or identity) to align corresponding positions in a sequence of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

As used herein, substantially identical to a product means sufficiently similar so that the property of interest is sufficiently unchanged so that the substantially identical product can be used in place of the product.

As used herein, it also is understood that the terms “substantially identical” or “similar” varies with the context as understood by those skilled in the relevant art.

As used herein, an allelic variant or allelic variation references any of two or more alternative forms of a gene occupying the same chromosomal locus. Allelic variation arises naturally through mutation, and can result in phenotypic polymorphism within populations. Gene mutations can be silent (no change in the encoded polypeptide) or can encode polypeptides having altered amino acid sequence. The term “allelic variant” also is used herein to denote a protein encoded by an allelic variant of a gene. Typically the reference form of the gene encodes a wildtype form and/or predominant form of a polypeptide from a population or single reference member of a species. Typically, allelic variants, which include variants between and among species typically have at least 80%, 90% or greater amino acid identity with a wildtype and/or predominant form from the same species; the degree of identity depends upon the gene and whether comparison is interspecies or intraspecies. Generally, intraspecies allelic variants have at least about 80%, 85%, 90% or 95% identity or greater with a wildtype and/or predominant form, including 96%, 97%, 98%, 99% or greater identity with a wildtype and/or predominant form of a polypeptide. Reference to an allelic variant herein generally refers to variations in proteins among members of the same species.

As used herein, “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for that gene or allele. When a subject has two different alleles of a gene, the subject is said to be heterozygous for the gene. Alleles of a specific gene can differ from each other in a single nucleotide or several nucleotides, and can include modifications such as substitutions, deletions and insertions of nucleotides. An allele of a gene also can be a form of a gene containing a mutation.

As used herein, species variants refer to variants in polypeptides among different species, including different mammalian species, such as mouse and human.

As used herein, modification is in reference to modification of a sequence of amino acids of a polypeptide or a sequence of nucleotides in a nucleic acid molecule and includes deletions, insertions, and replacements of amino acids and nucleotides, respectively. Methods of modifying a polypeptide are routine to those of skill in the art, such as by using recombinant DNA methodologies.

As used herein, isolated or purified polypeptide or protein or biologically-active portion thereof is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound.

The term substantially free of cellular material includes preparations of proteins in which the protein is separated from cellular components of the cells from which it is isolated or recombinantly-produced. In one embodiment, the term substantially free of cellular material includes preparations of enzyme proteins having less than about 30% (by dry weight) of non-enzyme proteins (also referred to herein as a contaminating protein), generally less than about 20% of non-enzyme proteins or 10% of non-enzyme proteins or less than about 5% of non-enzyme proteins. When the enzyme protein is recombinantly produced, it also is substantially free of culture medium, i.e., culture medium represents less than about or at 20%, 10% or 5% of the volume of the enzyme protein preparation.

As used herein, the term substantially free of chemical precursors or other chemicals includes preparations of enzyme proteins in which the protein is separated from chemical precursors or other chemicals that are involved in the synthesis of the protein. The term includes preparations of enzyme proteins having less than about 30% (by dry weight), 20%, 10%, 5% or less of chemical precursors or non-enzyme chemicals or components.

As used herein, synthetic, with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, production by recombinant means by using recombinant DNA methods means the use of the well known methods of molecular biology for expressing proteins encoded by cloned DNA.

As used herein, vector (or plasmid) refers to discrete elements that are used to introduce a heterologous nucleic acid into cells for either expression or replication thereof. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art.

As used herein, an expression vector includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, vector also includes “virus vectors” or “viral vectors.” Viral vectors are engineered viruses that are operatively linked to exogenous genes to transfer (as vehicles or shuttles) the exogenous genes into cells.

As used herein, operably or operatively linked when referring to DNA segments means that the segments are arranged so that they function in concert for their intended purposes, e.g., transcription initiates downstream of the promoter and upstream of any transcribed sequences. The promoter is usually the domain to which the transcriptional machinery binds to initiate transcription and proceeds through the coding segment to the terminator.

As used herein, the term assessing is intended to include quantitative and qualitative determination in the sense of obtaining an absolute value for the activity of a protease, or a domain thereof, present in the sample, and also of obtaining an index, ratio, percentage, visual or other value indicative of the level of the activity. Assessment can be direct or indirect and the chemical species actually detected need not of course be the proteolysis product itself but can for example be a derivative thereof or some further substance. For example, detection of a cleavage product of a complement protein, such as by SDS-PAGE and protein staining with Coomassie blue.

As used herein, biological activity refers to the in vivo activities of a compound or physiological responses that result upon in vivo administration of a compound, composition or other mixture. Biological activity, thus, encompasses therapeutic effects and pharmaceutical activity of such compounds, compositions and mixtures. Biological activities can be observed in in vitro systems designed to test or use such activities. Thus, for purposes herein a biological activity of a protease is its catalytic activity in which a polypeptide is hydrolyzed.

As used herein equivalent, when referring to two sequences of nucleic acids, means that the two sequences in question encode the same sequence of amino acids or equivalent proteins. When equivalent is used in referring to two proteins or peptides, it means that the two proteins or peptides have substantially the same amino acid sequence with only amino acid substitutions that do not substantially alter the activity or function of the protein or peptide. When equivalent refers to a property, the property does not need to be present to the same extent (e.g., two peptides can exhibit different rates of the same type of enzymatic activity), but the activities are usually substantially the same.

As used herein, a composition refers to any mixture. It can be a solution, suspension, liquid, powder, paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, “disease or disorder” refers to a pathological condition in an organism resulting from cause or condition including, but not limited to, infections, acquired conditions, genetic conditions, and characterized by identifiable symptoms. Diseases and disorders of interest herein include diabetes mellitus.

As used herein, “treating” a subject with a disease or condition means that the subject's symptoms are partially or totally alleviated, or remain static following treatment. Hence treatment encompasses prophylaxis, therapy and/or cure. Prophylaxis refers to prevention of a potential disease and/or a prevention of worsening of symptoms or progression of a disease. Treatment also encompasses any pharmaceutical use of a co-formulation of insulin and hyaluronan degrading enzyme provided herein.

As used herein, a pharmaceutically effective agent, includes any therapeutic agent or bioactive agents, including, but not limited to, for example, anesthetics, vasoconstrictors, dispersing agents, conventional therapeutic drugs, including small molecule drugs and therapeutic proteins.

As used herein, treatment means any manner in which the symptoms of a condition, disorder or disease or other indication, are ameliorated or otherwise beneficially altered.

As used herein, a therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

As used herein, the term “subject” refers to an animal, including a mammal, such as a human being.

As used herein, a patient refers to a human subject exhibiting symptoms of a disease or disorder.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, prevention or prophylaxis refers to methods in which the risk of developing disease or condition is reduced.

As used herein, a “therapeutically effective amount” or a “therapeutically effective dose” refers to the quantity of an agent, compound, material, or composition containing a compound that is at least sufficient to produce a therapeutic effect. Hence, it is the quantity necessary for preventing, curing, ameliorating, arresting or partially arresting a symptom of a disease or disorder.

As used herein, a therapeutically effective insulin dosage is the amount of insulin required or sufficient to achieve glycemic control. This amount can be determined empirically, such as by glucose or meal challenge. The compositions provided herein contain a therapeutically effective amount or concentration of insulin so that therapeutically effective dosages are administered.

As used herein, unit dose form refers to physically discrete units suitable for human and animal subjects and packaged individually as is known in the art.

As used herein, a single dosage formulation refers to a formulation for direct administration.

As used herein, an “article of manufacture” is a product that is made and sold. As used throughout this application, the term is intended to encompass a fast-acting insulin composition and hyaluronan degrading enzyme composition contained in the same or separate articles of packaging.

As used herein, fluid refers to any composition that can flow. Fluids thus encompass compositions that are in the form of semi-solids, pastes, solutions, aqueous mixtures, gels, lotions, creams and other such compositions.

As used herein, a “kit” refers to a combination of compositions provided herein and another item for a purpose including, but not limited to, reconstitution, activation, instruments/devices for delivery, administration, diagnosis, and assessment of a biological activity or property. Kits optionally include instructions for use.

As used herein, animal includes any animal, such as, but are not limited to primates including humans, gorillas and monkeys; rodents, such as mice and rats; fowl, such as chickens; ruminants, such as goats, cows, deer, sheep; pigs and other animals. Non-human animals exclude humans as the contemplated animal. The enzymes provided herein are from any source, animal, plant, prokaryotic and fungal. Most enzymes are of animal origin, including mammalian origin.

As used herein, a control refers to a sample that is substantially identical to the test sample, except that it is not treated with a test parameter, or, if it is a plasma sample, it can be from a normal volunteer not affected with the condition of interest. A control also can be an internal control.

As used herein, the singular forms “a,” “an” and “the” include plural referents unless the context clearly dictates otherwise. Thus, for example, reference to a compound, comprising “an extracellular domain” includes compounds with one or a plurality of extracellular domains.

As used herein, ranges and amounts can be expressed as “about” a particular value or range. About also includes the exact amount. Hence “about 5 bases” means “about 5 bases” and also “5 bases.”

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochemistry 11:1726).

### B. INSULIN THERAPY

Accelerating the absorption and action of prandial insulin products for both multidose injection (MDI) and continuous subcutaneous insulin infusion (CSII) administration is desired in order to more closely mimic the endogenous (i.e. natural) post-prandial insulin release of a nondiabetic subject. It has been shown that co-formulating or co-mixing fast acting insulin (e.g. an insulin analog) with a hyaluronan-degrading enzymes, such as PH20, acts to accelerate absorption and action compared to insulin alone when administered by subcutaneous infusion or pump infusion, and thereby result in improvements in glycemic control (see e.g. U.S. patent Pub No. US20090304665).

Also, continuous subcutaneous infusion (CSII) of an insulin also is a mechanism that is known to accelerate insulin exposure and/or action over the usual usage period (3-4) days of CSII infusion set (see e.g. Swan et al. (2009) Diabetes Care, 32:240-244; Liu et al. Diabetes Res. and Clin. Prac. (1991) 12:19-24; Olsson et al. Diabetic Medicine (1993) 10:477-80; and Clausen et al. Diabetes Tech Therapeutics (2009) 11:575-580). Previous studies, however, have demonstrated inconsistency in both exposure to and action of rapid acting analog insulin as insulin infusion set ages (Swan et al. (2009) Diabetes Care, 32:240-244; Clausen et al. Diabetes Tech Therapeutics (2009) 11:575-580). While a faster-in/faster-out absorption exists late in infusion set life, the insulin absorption is not consistent because early in infusion set life the insulin absorption that is observed is much less than occurs later in infusion set life. This results is a variability in insulin exposure upon CSII therapy, since insulin absorption only increases or accelerates later in infusion set life. For example, time to maximum insulin concentration has been observed to vary from 55±3 to 45±4 min (p=0.019) over 4 days of infusion set life. Correspondingly, onset of insulin action varied by 25% and duration of insulin action by 40 minutes across infusion set life.

This degree of variability in insulin exposure and action are meaningful confounders in the control of diabetes. Indeed, a single arm study of glucose control over infusion set use evaluated by continuous glucose monitoring has shown dramatic declines in glycemic control, with average daily glucose levels rising from 122.7 mg/dL to 163.9 mg/dL (p<0.05) after 5 days of infusion set use (Thethi et al. (2010) J. Diab. and its Complications, 24, 73-78). Consistent with the rise in mean daily glucose, the percentage of values in excess of 180 mg/dL rose from 14.5% to 38.3% (p<0.05). Also, it is found herein that the delivery of insulin alone by CSII also decreased total insulin action over time of infusion set life. The effect of this phenomena is variability to the patient in the insulin exposure profile.

Provided herein are continuous subcutaneous insulin infusion (CSII) dosing regime methods to minimize the effect of insulin acceleration across infusion set life (i.e. over time of infusion) in order to consistently deliver a super-fast acting insulin exposure and action profile over the duration of infusion set use. It is found herein that when an insulin co-formulated with a hyaluronan degrading enzyme (e.g. PH20) is infused by CSII, the CSII infusion acceleration phenomena is reduced, but not eliminated, while the loss of total insulin action is increased (see e.g. Example 2). To offset the loss of total insulin action while taking advantage of the more consistent effect of PH20 on insulin exposure and/or action, provided herein is a method whereby insulin administration is systematically increased over time in infusion set life, thereby improving glucose control over time by infusion pump therapy, including by both open-loop and closed-loop systems.

Also provided herein is a method to control insulin exposure and/or action, whereby the hyaluronan-degrading enzyme is administered at the initiation of infusion set use in a leading edge dosage regime prior to infusion with an insulin in a CSII therapy. The effect of the preadministration of a hyaluronan-degrading enzyme prior to infusion is a reduction in the variability of insulin exposure that occurs over time of infusion set life. As discussed elsewhere herein, it is believed that at the initiation of infusion, hyaluronan acts as a barrier to bulk fluid flow, thereby limiting the absorption of insulin. As the infusion set ages, the body naturally restores the hyaluronan barrier to bulk fluid flow over the course of infusion set use. By administering a hyaluronan-degrading enzyme prior to initiation of infusion with insulin, the initial barrier to bulk fluid flow is reduced. Hence, in the methods provided herein, the hyaluronan-degrading enzyme (e.g. PH20) can reduce the acceleration of insulin exposure and/or action over infusion set life and provide a more consistent delivery of a super-fast acting insulin that mimic the endogenous post-prandial insulin release of a nondiabetic subject.

1. Insulin, Diabetes and Existing Fast-Acting Insulin Therapies

Insulin is a naturally-occurring polypeptide hormone secreted by the pancreas. Insulin is required by the cells of the body to effectively take up and use glucose from the blood. Glucose is the predominant energy substrate to carry out cellular functions. In addition to being the primary modulator of carbohydrate homeostasis, insulin has effects on fat metabolism. It can change the ability of the liver and adipose tissue, among others, to release fat stores. Insulin has various pharmacodynamic effects throughout the body, including but not limited to increase in lipid synthesis, reduction in lipid breakdown, increase in protein synthesis, regulation of key enzymes and processes in glucose metabolism (including glucose uptake stimulation, glucose oxidation stimulation, increased glycogen synthesis and reduced glycogen breakdown).

Although insulin is secreted basally, usually in the range of 0.5 to 1.0 unit per hour, its levels are increased after a meal. After a meal, the pancreas secretes a bolus of insulin in response to a rise in glucose. Insulin stimulates the uptake of glucose into cells, and signals the liver to reduce glucose production; this results in a return of blood glucose to normal levels. In normal adults, there are two phases of insulin release in response to a meal. The early phase is a spike of insulin release that occurs within 2-15 minutes of eating. The late phase release extends about 2 hours. The early phase is responsible for shutting down hepatic glucose production, thereby reducing blood glucose levels and sensitizing or signaling peripheral tissues to increase glucose uptake. In muscle, large amounts of glucose are stored as glycogen. Some of the glycogen is broken down into lactate, which circulates to the liver and can be converted back into glucose and stored as glycogen. Between meals the liver breaks down these glycogen stores to provide glucose to the brain and other tissues.

Diabetes results in chronic hyperglycemia due to the inability or reduced ability of the pancreas to produce adequate amounts of insulin or due to the inability or reduced ability of cells to synthesize and/or release the insulin required. In diabetics, the effectiveness of the above described first-phase response is decreased or absent, leading to elevated postprandial glucose levels. For example, blood glucose area under the curve (AUC) during the first four postprandial hours (i.e. first four hours after eating), is 2.5 to 3.0 times greater in diabetics than in non-diabetics. Postprandial glucose excursions contribute to overall hyperglycemia and elevated HbAlc levels, and these excursions are the primary contributors to HbAlc elevations seen in early stages of Type 2 diabetes.

Many diabetic patients require treatment with insulin when the pancreas produces inadequate amounts of insulin, or cannot use the insulin it produces, to maintain adequate glycemic control. Insulin has been administered as a therapeutic to treat patients having diabetes, including, for example, type 1 diabetes, type 2 diabetes and gestational diabetes, in order to mimic the endogenous insulin response that occurs in normal individuals. Insulin also has been administered to critically ill patients with hyperglycemia to control blood glucose levels.

Insulin replacement therapy involves both basal and bolus insulin replacement. Basal insulin replacement, or background insulin, is used to control blood sugar while fasting, for example, overnight or between meals, and is usually administered at a constant day to day dose. Bolus insulin replacement accounts for carbohydrates, i.e., food intake, and also high blood sugar correction, also known as insulin sensitivity factor. The bolus dose for food coverage is prescribed as an insulin to carbohydrate ratio, or carbohydrate coverage ratio. The insulin to carbohydrate ratio represents how many grams of carbohydrate are covered or disposed of by I unit of insulin. Generally, one unit of rapid-acting insulin will dispose of 12-15 grams of carbohydrate. This range can vary from 4-30 grams or more of carbohydrate depending on an individual's sensitivity to insulin. Insulin sensitivity can vary according to the time of day, from person to person, and is affected by physical activity and stress. The bolus dose for high blood sugar correction is defined as how much one unit of rapid-acting insulin will drop the blood sugar. Generally, to correct a high blood sugar, one unit of insulin is needed to drop the blood glucose by 50 mg/dl. This drop in blood sugar can range from 15-100 mg/dl or more, depending on individual insulin sensitivities, and other circumstances. Overweight patients require higher doses of insulin because of greater insulin resistance and deficiency. Dose adjustments can also be required if the patient is taking medications that can affect carbohydrate metabolism or responses to insulin. Liver or renal disease can also affect the pharmacokinetics of insulin. In addition, exercise, illness, stress, aberrant eating patterns, alcohol, and travel may also necessitate dose adjustments.

Algorithms used to estimate insulin doses vary and are known to one of skill in the art (see, e.g., Hirsch et al., (2005) Clinical Diabetes 23:78-86; Global Guideline for Type 2 Diabetes, Chapter 10: Glucose control: insulin therapy, International Diabetes Federation, (2005) pp. 39-42; Zisser et al., (2009) J Diabetes Sci Technol 3(3):487-491). A starting regimen is determined primarily by the degree of hyperglycemia as measured by blood glucose monitoring and the AlC value. Body weight is also used to calculate the appropriate starting insulin dose. Blood glucose monitoring is essential for evaluation of a dosage regimen. Typically, at least one fasting and one postprandial blood glucose value are measured and recorded. The frequency and timing of blood glucose testing depends primarily on the insulin regimen. Those using multiple daily injection (MDI) therapy often need to check the blood glucose level before each meal, occasionally 2 hours postprandial, and at bedtime each day. Finger sticks can be done before and after one meal to determine the impact of the pre-meal insulin dose, and adjustments can be made accordingly. The meal selected should vary so that at the end of the assessment period, each meal is studied at least once. Testing overnight and the next morning provides information concerning the impact of the basal insulin.

To calculate a basal insulin dose, or background insulin dose, one must first estimate or calculate a total daily insulin dose. The total daily insulin requirement (in units) is generally defined as the patient's weight in pounds divided by 4, or alternatively, the patients weight in kilograms multiplied by 0.55. For example, if a patient weights 160 pounds, the total daily insulin requirement would be 40 units of insulin per day (160÷4). Patients with insulin sensitivity may require a higher total daily insulin dose, or alternatively, a patient that is sensitive to insulin may require a lower total daily insulin dose. The basal insulin dose is then calculated based on the total daily insulin dose (TDI). The basal insulin dose is approximately 40-50% of the total daily insulin dose. Thus, for a patient above with a TDI of 40 units, the basal or background insulin dose is 20 units.

A carbohydrate coverage ratio, or the grams of carbohydrate covered by one unit of insulin, is calculated by the formula 500÷Total Daily Insulin Dose. Thus, if your TDI is 40 units, your carbohydrate coverage ratio is 12 g carbohydrates per unit insulin (equal to 500÷40). A high blood sugar correction factor, or the amount 1 unit of insulin will decrease blood sugar (in mg/dl) is calculated by dividing 1800 by the Total Daily Insulin Dose. Thus, if your TDI is 40 units, your correction factor is 45 mg/dl (equal to 1800÷40).

To calculate a bolus insulin dose for carbohydrates, or food intake, the total grams of carbohydrates in the meal is divided by the grams of carbohydrate disposed by 1 unit of insulin, i.e., carbohydrate coverage ratio described above. For example, 1 unit of a rapid-acting analog can be given for every 10 to 15 grams of carbohydrate consumed. Therefore a meal containing 90 grams of carbohydrate would require a bolus dose of 6 units insulin (1:15 ratio). To calculate a bolus insulin dose for high blood sugar correction, one takes the difference between actual blood sugar and target blood sugar (i.e., the actual blood sugar minus the target blood sugar), and divides by a correction factor. In general, 1 unit of insulin will drop your blood sugar 50 points (mg/dl) and therefore the high blood sugar correction factor is 50. Thus, if a patient's measured blood glucose level was 220 mg/dl and his pre-meal blood sugar target is 120 mg/dl, the dose required for high blood sugar correction is (220-120 mg/dl)÷50, resulting in a dose of 2 units of insulin. Typically, patients using MDIs or an insulin pump can adjust the mealtime insulin dose based on the estimated carbohydrate content of a meal as well as a blood glucose reading. For example, assuming a patient is about to eat meal which is estimated to contain 90 grams of carbohydrate and the patient's premeal blood glucose target is 100 mg/dL, but the measured blood glucose level was 200 mg/d, the bolus insulin dose can be determined. Thus, using an insulin:carbohydrate ratio of 1:15, the patient will take 6 units of insulin aspart to cover the 90 grams of carbohydrate (90 grams carbohydrate/15) plus another 2 units of insulin aspart to correct being 100 mg/dL over the target glucose level. His total bolus insulin dose will be 8 units.

Different sources of insulins are used depending on the patient need. Commercial insulin preparations can be classified depending on their duration of activity (see e.g., DeFelippis et al. (2002) Insulin Chemistry and Pharmacokinetics. In Ellenberg and Rifkin's Diabetes Mellitus (pp. 481-500) McGraw-Hill Professional). For example, insulin is provided in fast-acting formulations, as well as intermediate- or long-acting formulations, the latter two classifications being referred to herein as basal-acting insulins. The fast-acting forms have a rapid onset, typically exhibiting peak insulin levels in 2-3 hours or less, and no more than four hours. Hence, fast-acting forms of insulin are used in prandial glucose regulation. Other forms of insulin include intermediate-acting, which reach peak insulin concentration at approximately 4-12 hours following subcutaneous administration, and long-acting insulins that reach a relatively modest peak and have a maximum duration of action of 20-30 hours. The intermediate- and long-acting forms are often composed of amorphous and/or crystalline insulin preparations, and are used predominantly in basal therapies.

The goal of prandial administration of fast-acting insulin compositions is to attain a stable blood glucose level over time by parenteral administration of the fast-acting insulin before, during or soon after mealtime. In this way, blood levels of insulin are temporarily elevated to (a) shut down hepatic glucose production and (b) increase glucose uptake; thus maintaining glycemic control during the elevation in blood glucose associated with meal digestion.

Recombinant human insulin (also called regular insulin; e.g., Humulin® R insulin) is used for self administration by injection prior to meal time. Unfortunately, recombinant human insulin must be dosed by injection approximately one half hour or more prior to meal time in order to insure that a rise in blood glucose does not occur unopposed by exogenous insulin levels. One of the reasons for the slow absorption of recombinant human insulin is because insulin forms hexameric complexes in the presence of zinc ions both in vivo and in vitro. Such hexameric zinc-containing complexes are more stable than monomeric insulin lacking zinc. Upon injection, these insulin hexamers must dissociate into smaller dimers or monomers before they can be absorbed through capillary beds and pass into the systemic circulation. The dissociation of hexamers to dimers and monomers is concentration-dependent, occurring only at lower concentrations as the insulin diffuses from the injection site. Thus, a local insulin depot exists at the injection site, providing an initial high concentration of hexameric insulin at the site of injection that cannot be absorbed until the insulin concentration decreases (Soeborg et al., (2009) Eur. J. Pharm. Sci. 36:78-90). As the insulin slowly diffuses from the injection site, the insulin concentration lowers as the distance from the injection site increases, resulting in dissociation of the hexamers and absorption of the insulin monomers and dimers.

Thus, although dispersal of hexameric insulin complexes occurs naturally in the body, it can take some time to occur, delaying the systemic availability of insulin. Further, because of this concentration-dependent absorption, higher insulin concentrations and higher doses are absorbed more slowly (Soeborg et al., (2009) Eur. J. Pharm. Sci. 36:78-90).

Since insulin in monomeric form is absorbed more rapidly, while insulins in the hexameric state are more stable, fast-acting analog (also called rapid-acting) forms of insulin have been developed that exhibit a faster dissociation from hexameric to monomeric upon administration. Such insulins are modified, such as by amino acid change, to increase the dissociation rate, thereby imparting more rapid pharmacodynamic activity upon injection. As described in Section D, fast-acting analog forms of insulin include but are not limited to, insulin glulisine, insulin aspart, and insulin lispro.

Fast-acting forms of insulins, including fast-acting analogs, have a delay in absorption and action, and therefore do not approximate endogenous insulin that has an early phase that occurs about 10 minutes after eating. Thus, such formulations do not act quickly enough to shut off hepatic glucose production that occurs shortly after this first phase of insulin release. For this reason, even the fast-acting insulin analog preparations must be given in advance of meals in order to achieve any chance of desired glycemic control. Although it is easier to estimate time of eating within 15 minutes than within 30-60 minutes required for regular insulin, there is a risk that a patient may eat too early or too late to provide the best blood glucose control.

Further, one of the main side effects of treatment with any insulin therapy, including fast-acting insulin therapies, is hypoglycemia. Hypoglycemia is defined as low blood glucose and is associated with a variety of morbidities that may range from hunger to more bothersome symptoms such as tremor, sweating, confusion or all the way to seizure, coma and death. Hypoglycemia can occur from failure to eat enough, skipping meals, exercising more than usual or taking too much insulin or using an prandial insulin preparation that has an inappropriately long duration of exposure and action. For example, since many fast-acting insulin therapies must be given before a meal, there is a risk that a patient may forego or skip the meal, leading to hypoglycemia. Additionally, upon administration of a fast-acting insulin, serum insulin levels and insulin action (measured, for example, as glucose infusion rate (GIR)) typically remain elevated after the prandial glucose load has abated, threatening hypoglycemia. Attempts to better control peak glucose loads by increasing insulin dose further increases this danger. Also, because postprandial hypoglycemia is a common result of insulin therapy, it often causes or necessitates that patients eat snacks between meals. This contributes to the weight gain and obesity often associated with insulin therapies.

Previous studies of insulin coadministered with a hyaluronan-degrading enzyme (e.g. PH20 such as rHuPH20) have demonstrated insulin pharmacokinetics that better replicate the natural insulin response to a meal in healthy individuals (see e.g. U.S. patent Pub No. US20090304665; Vaughn et al. (2009) Diabetes Technol. Ther., 11:345-52; Muchmore and Vaughn (2010) J. Diabetes Sci. Technol., 1:19-28). Specifically, coadministration of insulin with PH20 accelerates the onset of insulin action (early t_{50%max}), the time of peak insulin concentration (tₘₐₓ), and the offset of insulin action (late t_{50%max}). PH20 coadministration also increases the peak insulin concentration, increases early insulin exposure, and reduces late postprandial insulin exposure. In healthy volunteers, this acceleration of insulin exposure results in accelerated glucose metabolism, as measured by glucose infusion rates during a euglycemic clamp. In subjects with Type I and Type 2 diabetes mellitus, the acceleration of insulin exposure has been shown to reduce postprandial hyperglycemia, as measured by peak blood glucose, two-hour post-prandial glucose, and total area of glucose excursions >150 mg/d occurring in response to a standardized liquid test meal.

2. Continuous Subcutaneous Infusion (CSII)

Continuous subcutaneous insulin infusion (CSII) has been used clinically for the treatment of diabetes over the last three decades and closed loop “artificial pancreas” systems using CSII for the efferent control component are under development. CSII permits management control of insulin therapy that cannot be achieved by subcutaneous injections. For example, insulin pumps can account for residual insulin action in the accompanying software to prevent hypoglycemia related to multiple bolus doses given over a short period.

CSII pump therapy is associated with increasing glucose variability as the infusion site ages, which can be a problem in management (Swan et al. (2009) Diabetes Care, 32:240-244). For example, prolonged use of an infusion site (e.g. up to 4 days) results in earlier peak action and shorter duration of action of a standard bolus dose, which is similar for different fast-acting insulin analogs. This effect can contribute to day-to-day variability and plasma glucose liability in diabetic patients. This effect has been observed in several studies.

For example, a paper by Liu et al. (Diabetes Res. and Clin. Prac. (1991) 12:19-24) demonstrated acceleration of insulin exposure without any change in total insulin exposure occurring between day 1 and day 4 of insulin infusion set use. Notably, the test performed on Day I was conducted immediately (within 10 minutes) after changing the infusion set. The change in insulin exposure timing was associated with a faster and greater decline in blood glucose levels following the delivery of a bolus dose (1 unit/10 kg body weight) on Day 4 as compared to Day 1. The conclusion was that insulin absorption rate increased as infusion sets age, and that the change in absorption is associated with more rapid insulin action as assessed by blood glucose decline following insulin bolus administration and subsequent meal.

A similar study was reported by Olsson et al. (Diabetic Medicine (1993) 10:477-80) and failed to show any meaningful difference in the timing of insulin exposure when comparing studies performed on Days 1, 3 and 5 of infusion set use. As in the study by Liu et al. total insulin exposure was comparable across the study days. Notably, in this study, the insulin bolus on Day 1 was administered approximately 12 hours after changing the infusion set. The authors assessed insulin action by following blood glucose levels after a standard meal given after the daily morning bolus of insulin, which were found to be fairly constant. There were no statistically significant differences in blood glucose although it was noted that there was a trend for blood sugar to progressively rise more quickly on Days 1, 3 and 5, respectively, and the fasting blood glucose tended to be greater on Day 5 than Days 1 or 3.

In the more recent study by Swan et al. (Diabetes Care (2009) 32:240-244), insulin action on Day 1 (12 hours after infusion set change) was compared to Day 4 (84 hours after infusion set change). Insulin action was assessed by measuring glucose infusion rate over time that was required to maintain euglycemia following a bolus dose of insulin. Insulin blood levels were not measured in this study. The authors found a significant acceleration of insulin action that occurred as the infusion set aged. The authors concluded that total insulin action, measured by total glucose infused during the experiment, was not different when comparing Day 4 to Day 1, although the data did show a modest but non-significant trend for reduced insulin action when comparing Day 4 to Day 1. In contrast to the previous two studies the infusion site was gluteal, not abdominal, and the subjects were adolescents.

A study reported by Clausen et al. (Diabetes Tech Therapeutics (2009) 11:575-580) assessed subcutaneous blood flow and insulin pharmacokinetics delivered by CSII in healthy male volunteers daily for four days (Days 0-3). The insulin bolus was given 90 minutes after infusion set insertion; after the bolus was delivered the subjects received continuous infusion of saline until the next scheduled bolus. The results confirmed those of Liu et al., with progressive acceleration of insulin exposure without change in total exposure over the life of the infusion set. Insulin action assessments were not performed.

These findings generally support the idea that insulin exposure and action accelerate systemically over the life of an infusion set. Generally, after being infused or injected into subcutaneous tissue, insulin builds up a depot, which ultimately diffuses through the interstitial space to the vascular bed where hexamer-dissociated monomers or dimers are absorbed into the vascular bed. The reasons for the earlier onset and shorter duration of bolus doses at later times of infusion can be due to a variety of factors, such as increased blood flow around the infusion site due to changes in the vascular microenvironment (e.g. caused by inflammatory reactions at the infusion site), loss of insulin due to precipitation in the set or partial occlusion of the infusion set by insulin (Swan et al. (2009) Diabetes Care, 32:240-244). Also, the transport of insulin across the membrane at early times also may be limited by building up of a depot of insulin, diffusion capacity or blood flow. For example, the acceleration can be due to the hyaluronan barrier to bulk fluid flow at the onset of infusion. This barrier to bulk fluid flow may not exist, or is compensated for by the other factors, at later infusion times. In the methods provided herein, the differences in insulin exposure and/or action over time can be minimized by a leading edge treatment, whereby a hyaluronan-degrading enzyme is administered at the initiation of infusion set use, followed by CSII with insulin alone or an insulin-PH20 combination or co-formulation. At later times over the course of the infusion set use, the body naturally restores the hyaluronan barrier to bulk fluid flow so as to reduce the acceleration.

Both open loop and closed loop systems benefit from the development of insulin preparations containing PH20, which have a reduced lag time between injection and action. The presence of PH20 in combination with insulin reduce the acceleration of insulin exposure over time of infusion set. Dosing regimes using PH20 and/or insulin further reduce variability in the acceleration of insulin exposure, and thereby control the variability in insulin exposure occurring over time of infusion. Provided herein are CSII dosage regime methods that minimize the effect of insulin acceleration across infusion set life (i.e. over time of infusion) in order to consistently deliver a super-fast acting insulin exposure and action profile over the duration of infusion set use. The methods of controlling insulin exposure and/or action can be used in CSII methods and uses for treating diabetes and/or for more consistently controlling blood glucose levels in a subject.

### C. CONTINUOUS SUBCUTANEOUS INFUSION (CSII) METHODS OF INSULIN WITH A HYALURONAN-DEGRADING ENZYME

Provided herein are continuous subcutaneous infusion (CSII) dosage regimen methods for controlling blood glucose levels in a subject. The methods can be used for treating a patient that has diabetes or other insulin-associated disease or condition. The methods provided herein are based on the finding that a dosage regimen including a hyaluronan-degrading enzyme consistently delivers an ultra-fast insulin exposure and action profile over the duration of infusion set use. Hence, the methods herein using a hyaluronan-degrading enzyme, in particular in a leading edge administration, can be used to minimize the difference in insulin absorption over time of insulin infusion in a subject.

In any of the methods herein, if the continuous subcutaneous infusion is disrupted or halted, for example because of pump shut-off due to pump failure, catheter occlusion or user error, the insulin action can stop faster than with a slower acting insulin. This could accelerate the time of hyperglycemia and eventually diabetic ketoacidosis. Hence, in any of the methods provided herein is an optional step, as necessary, of administering a long-acting (a.k.a. basal) insulin at an appropriate interval. Typically, the long-acting insulin would be one with a duration of action of at least about 12 hour. Exemplary long-acting insulins known in the art include, but are not limited to, Levemir, detemir, NPH insulin or degludec. The long-acting insulin can be administered from about between to about 5% to 50% of the patients total daily insulin dose, such as about ⅓ or 33% of the patients total daily insulin dose. The basal insulin can be delivered at an appropriate interval, such as at least once per infusion set depending on the duration of action of the particular insulin and patient preference. For example, the basal insulin can be delivered at least once or twice per week or at least once or twice per day.

1. Dosage Regimen Methods

a. Leading Edge

In one example, the methods provided herein include administering a hyaluronan-degrading enzyme, such as a hyaluronidase for example a PH20, to a subject prior to initiation of CSII of a fast-acting insulin. The hyaluronan in the interstitial space serves as a barrier to bulk fluid flow, thereby accounting for the slower rate of action of insulin exposure at the onset of infusion. This barrier to bulk fluid flow may not exist, or is compensated for by other factors, at later infusion times. Hence, as shown herein, over the course of infusion set there is an accelerated action of insulin late in infusion set life compared to early times of infusion, such that insulin action late in infusion set life exhibits a super-fast acting insulin response. Over the lifetime of the infusion set, this renders insulin action and absorption variable and inconsistent. In the methods provided herein, the differences in insulin exposure and/or action over time can be minimized by administering a hyaluronan-degrading enzyme at or near the initiation of infusion set use, followed by CSII with insulin alone or an insulin-PH20 super-fast action composition. For example, the hyaluronan-degrading enzyme is administered by a leading edge treatment. At later times over the course of the infusion set use, the body naturally restores the hyaluronan barrier to bulk fluid flow so as to reduce the difference in insulin acceleration as the infusion set ages. This reduces or minimizes the variability in insulin exposure and action that occurs in a patient over the course of CSII therapy.

In the method, a composition containing a hyaluronan-degrading enzyme is administered to a subject in a therapeutically effective amount sufficient to catalyze the hydrolysis of hyaluronic acid to increase tissue permeability. The amount of hyaluronan-degrading enzyme is an amount that effects an ultra-fast insulin response at the outset of infusion life. After administration of the hyaluronan-degrading enzyme, a fast-acting insulin is delivered to the subject using CSII. By practice of the continuous subcutaneous insulin infusion method, the difference in insulin absorption is minimized or reduced over the course of infusion set life. Hence, also provided herein are uses, processes or compositions contain a hyaluronan-degrading enzyme for use for minimizing the difference in insulin absorption that occurs over a course of continuous subcutaneous insulin infusion (CSII).

The particular amount and dosage regimen of insulin, including basal rate and bolus doses, that is delivered by CSII therapy is in accord with a patient-specific protocol that is dependent on the particular characteristics and needs of the patient. CSII therapy is well-known to one of skill in the art (Boland et al. (1999) Diabetes Care, 22:1779-1784). It is well within the skill of a skilled physician to treat a patient using CSII in accord with known and existing protocols and recommendations. Depending on the particular protocol and continuous infusion device that is used, the CSII therapy can be effected by infusion of insulin, generally via a pump, such as an open-loop or closed-loop pump. Typically, the CSII is performed for a predetermined interval that matches the infusion set life or performance of the continuous infusion device that is being used. For insulin pumps that contain an infusion set that contains a tubing system and insertion device such as a cannula, the interval is generally only several days, such as every 2-4 days. For example, the infusion set is replaced every 2-4 days. In one example, the infusion set is replaced twice weekly.

In such methods, any hyaluronan-degrading enzyme, such as any described in Section E below, can be used. In examples herein, the amount of hyaluronan-degrading enzyme that is administered to catalyze the hydrolysis of hyaluronic acid to increase tissue permeability can be determined empirically. The activity of a hyaluronan degrading enzyme can be assessed using methods well known in the art. For example, the USP XXII assay for hyaluronidase determines activity indirectly by measuring the amount of undegraded hyaluronic acid, or hyaluronan, (HA) substrate remaining after the enzyme is allowed to react with the HA for 30 min at 37° C. (USP XXII-NF XVII (1990) 644-645 United States Pharmacopeia Convention, Inc, Rockville, Md.). A Hyaluronidase Reference Standard (USP) or National Formulary (NF) Standard Hyaluronidase solution can be used in an assay to ascertain the activity, in units, of any hyaluronidase. In one example, activity is measured using a microturbidity assay or a microtiter assay using a biotinylated hyaluronic acid (see e.g. Frost and Stern (1997) Anal. Biochem. 251:263-269, U.S. Pat. Publication No. 20050260186). Other assays also are known (see e.g. see e.g. Delpech et al., (1995) Anal. Biochem. 229:35-41; Takahashi et al., (2003) Anal. Biochem. 322:257-263). The ability of a hyaluronan degrading enzyme to act as a spreading or diffusing agent to thereby increase permeability also can be assessed. For example, trypan blue dye can be injected subcutaneously with or without a hyaluronan degrading enzyme into the lateral skin on each side of nude mice. The dye area is then measured, such as with a microcaliper, to determine the ability of the hyaluronan degrading enzyme to act as a spreading agent (U.S. Pat. Pub. No. 20060104968). Similar experiments can be performed in other subjects.

Typically, the hyaluronan-degrading enzyme is administered in an amount that is functionally equivalent to between or about between 0.5 Units to 500 Units, 1 Unit to 200 Units, 5 Units to 150 Units, 10 Units to 150 Units, 50 Units to 150 Units or 1 Unit to 50 Units. For example, the hyaluronan-degrading enzyme is administered in an amount that is at least 1 Unit, 5 Units, 10 Units, 50 Units, 100 Units, 150 Units, 200 Units, 300 Units, 400 Units, 500 Units or more. In other examples, the hyaluronan-degrading enzyme is administered in an amount that is between or about between 1 ng to 10 μg, 8 ng to 2 μg, 20 ng to 1.6 μg, 80 ng to 1.25 μg or 200 ng to 1 μg. For example, the hyaluronan-degrading enzyme is administered in an amount that is at least 1 ng, 8 ng, 80 ng, 1.0 μg 1.25 μg, 1.6 μg, 2 μg, 3 μg, 4 μg, 5 μg, 6 μg, 7 μg, 8 μg, 9 μg, 10 μg or more. The volume of hyaluronan-degrading enzyme that is administered is generally 0.1 mL to 50 mL, such as 0.5 mL to 5 mL, generally between or about between 0.5 mL to 2.0 mL such as at least or about or 0.20 mL, 0.50 mL, 1.0 mL, 1.5 mL, 2.0 mL, 3.0 mL, 4.0 mL, 5.0 mL, 6.0 mL, 7.0 mL, 8.0 mL, 9.0 mL, 10.0 mL or more, for example at least or about at least or 1.0 mL.

In the methods herein, the hyaluronan-degrading enzyme typically is administered immediately before the initiation of CSII. Generally, however, it is only administered one time during the interval of infusion set life. Thus, in the methods herein, the hyaluronan-degrading enzyme is administered once at the initiation of CSII. Typically, after the end of each interval, the infusion set is replaced and the steps of administering a hyaluronan-degrading enzyme to a subject is repeated. For example, the hyaluronan-degrading enzyme can be administered sequentially, simultaneously or intermittently from the fast-acting insulin composition delivered by CSII over the course of infusion set intervals.

In particular examples, in each infusion set interval, the hyaluronan-degrading enzyme is administered prior to initiation of infusion in a leading dosage regimen. Then, following administration of the hyaluronan-degrading enzyme, a fast-acting insulin is delivered to the subject using CSII. The hyaluronan-degrading enzyme can be administered between or about between or approximately 10 seconds to 1 hour prior to initiation of infusion, 30 seconds to 30 minutes prior to initiation of infusion, 1 minute to 15 minutes prior to initiation of infusion, 1 minute to 12 hours prior to initiation of infusion, such as 5 minutes to 6 hours prior to initiation of infusion, 30 minutes to 30 hours prior to initiation of infusion, or 1 hour prior to initiation of infusion of a fast-acting insulin by CSII. Typically, the hyaluronan-degrading enzyme is administered no more than 2 hours before initiation of infusion of a fast-acting insulin by CSII. In other words, the hyaluronan-degrading enzyme is administered within 2 hours prior to initiation of infusion of a fast-acting insulin. For example, the hyaluronan-degrading enzyme is administered at least 10 seconds, at least 30 seconds, at least 1 minute, at least 2 minutes, at least 3 minutes, at least 5 minutes, at least 10 minutes, at least 20 minutes, at least 30 minutes, at least 40 minutes, at least 50 minutes, at least 1 hour or at least 2 hours prior to infusion of a fast-acting insulin analog.

In other examples, in each infusion set interval, the hyaluronan-degrading enzyme is administered simultaneously or near simultaneously with initiation of CSII. For example, the hyaluronan-degrading enzyme can be administered between or about between 0 to 1 minutes before initiation of infusion or between or about between 0 to 1 minutes after initiation of infusion.

It is understood that in some examples, a hyaluronan-degrading enzyme, such as a hyaluronidase for example a PH20, can be administered to a subject immediately after initiation of CSII of a fast-acting insulin. In such examples, the timing of administration of the hyaluronan-degrading enzyme is such that it sufficiently effects increased insulin absorption early in infusion set life, thereby decreasing the variability that occurs in patients undergoing CSII therapy in the absence of administration of a hyaluronan-degrading. Thus, while the administration of the hyaluronan-degrading enzyme does not precede infusion of insulin, the hyaluronan-degrading enzyme still effects a leading edge effect because it is able to remove hyaluronan to permit increased absorption of insulin early in infusion set life.

Thus, in further examples, in each infusion set interval, the hyaluronan-degrading enzyme can be administered after initiation of infusion. Thus, prior to administration of the hyaluronan-degrading enzyme, a fast-acting insulin is delivered to the subject using CSII. The hyaluronan-degrading enzyme can be administered between or about between 1 minute to 12 hours after initiation of infusion, such as between or about between 5 minutes to 6 hours after initiation of infusion, between or about between 30 minutes to 3 hours after initiation of infusion, or between or about between 1 hour to 2 hours after initiation of infusion. Typically, in such examples, the hyaluronan-degrading enzyme is administered no more than 2 hours after initiation of infusion of a fast-acting insulin by CSII.

The hyaluronan-degrading enzyme can be administered by any suitable route, such as, for example, parenteral administration, including subcutaneous, intramuscular, intraperitoneal, intravenous, and intradermal administration. The hyaluronan-degrading enzyme also can be administered intravenously. Typically, the hyaluronan-degrading enzyme is administered subcutaneously. The hyaluronan-degrading enzyme can be administered at or near the site of infusion of the fast-acting insulin. In some examples, the hyaluronan-degrading enzyme is administered through the same injection site as the CSII of fast-acting insulin. In other examples, the hyaluronan-degrading enzyme is administered at a different injection site than the CSII of a fast-acting insulin.

Any fast-acting insulin, such as any described in Section D below, can be used in the methods herein for delivery by CSII. Typically, the reservoir contains a fast-acting insulin composition that contains an amount of a fast-acting insulin that is between or about between 10 U/mL to 1000 U/mL, 50 U/mL to 500 U/mL, 100 U/mL to 250 U/mL, for example at least or about at least or 25 U/mL, 50 U/mL, 100 U/mL, 200 U/mL, 300 U/mL, 400 U/mL, 500 U/mL or more, such as at least or about at least 100 U/mL. In some example, the amount of insulin in the composition is between or about between 0.35 mg/mL to 35 mg/mL, 0.7 mg/mL to 20 mg/mL, 1 mg/mL to 15 mg/mL, 5 mg/mL to 10 mg/mL, such as at least or about at least 0.5 mg/mL, 1 mg/mL, 1.5 mg/mL, 2.0 mg/mL, 3.0 mg/mL, 4.0 mg/mL, 5.0 mg/mL, 10.0 mg/mL, 15 mg/mL, 20 mg/mL, 30 mg/mL or more. Generally, the fast-acting insulin is a fast-acting insulin analog (also termed rapid-acting analog). In some examples, the fast-acting insulin that is delivered by CSII is a super fast-acting insulin composition that contains a fast-acting insulin or fast-acting insulin analog and a hyaluronan-degrading enzyme sufficient to render the composition super-fast acting (published as U.S. publication No. US20090304665). Super fast-acting insulin compositions are described herein below in Section F. In further examples, the super fast-acting insulin compositions are stable compositions that are stable for at least 3 days at 32° C. to 40° C. as described further below and in provisional application No. 61/520,962.

Any continuous infusion device can be used in the methods herein to deliver a fast-acting insulin by CSII. Generally, the continuous insulin infusion device includes an insulin pump, a reservoir containing the fast-acting insulin or super-fast acting insulin composition and an infusion set for subcutaneous infusion of the device. The device can be an open loop or closed-loop device. Exemplary insulin pumps and other insulin delivery devices for continuous insulin infusion are described in Section C.2 below.

In an exemplary example of the method, a new pump reservoir of a continuous infusion device is filled with an effective concentration of a fast-acting insulin, for example a fast-acting insulin analog composition. The amount of insulin in the composition is generally about or at least or 100 U/mL. The patient is then inserted with a new infusion set, typically at an abdominal site. The insertion needle or cannula is affixed with an adhesive pad. The infusion set is then attached to the filled pump reservoir. The infusion set is then primed with insulin. Prior to initiating the infusion of insulin via the pump into the patient, a 1.0 mL hyaluronan-degrading enzyme, such as a hyaluronidase for example a PH20 (e.g. rHuPH20) composition containing enzyme that is in an amount that is at least or about or 100 U/mL, 150 U/mL, 200 U/mL, 300 U/mL, 400 U/mL, 500 U/mL or 600 U/mL is injected into the patient at or near the infusion site. For example, the hyaluronan-degrading enzyme is introduced via a syringe or other similar device or tube typically containing a needle for injection. The other device can be adaptor that is compatible for insertion through the cannula or infusion site. Generally, the enzyme is administered into the same injection site and via the same cannula as will be used for the infusion. The hyaluronan-degrading enzyme is administered slowly, generally not less than 20 seconds to 30 seconds, to the patient. Immediately after injection of a hyaluronan-degrading enzyme, the hyaluronan-degrading enzyme infusion set is removed from the cannula or other similar insertion device and replaced with the insulin-containing pump/infusion set. The pump is then programmed to deliver a fixed prime infusion depending on the size of the cannula (e.g. 0.2 U to 1.0 U depending on the size of the cannula; for example, 0.4 U for a 6 mm cannula and 0.6 U for a 9 mm cannula) and then a predetermined patient-specific programmed basal infusion rate of insulin is continuously delivered. Hence, in exemplary methods herein, the hyaluronan-degrading enzyme is administered generally within or approximately or about 5 seconds to 20 minutes, such as 1 minute to 15 minutes of infusion of insulin.

b. Method to Ameliorate Total Insulin Action

It is found herein that when administering a super-fast acting insulin composition in a CSII dosage regimen that there is a decreased total insulin action over the life of the infusion set. This decrease in total insulin action over time of infusion set is greater in super-fast acting insulin formulations that contain a hyaluronan-degrading enzyme than in fast-acting formulations. Systemically increasing insulin administration over time will offset the loss of insulin action and improve glucose control by both open-loop and closed-loop control. Hence, methods are provided herein whereby the basal or bolus dose of insulin in a super-fast acting insulin composition is increased over the life of an infusion set in order to compensate for the observed reduction in total insulin effect seen over time.

Provided herein is a continuous subcutaneous insulin infusion (CSII) dosage regimen method for controlling blood glucose that provides for a more consistent ultra-fast insulin profile over the course of the infusion set. In such examples, CSII is performed to deliver a super-fast acting insulin composition to a patient in accord with a programmed basal rate and bolus dose of insulin. Section F describes super-fast acting insulin compositions. In some examples, a stable co-formulation is employed in the method. Any insulin delivery device for continuous infusion can be employed in the method, including a device that provides a closed-loop or open-loop system. Exemplary of such devices are described in Section C.2 below.

In the method, during the course of the dosage regimen, the amount of super-fast acting insulin, basal and/or bolus, that is administered is increased at least 1% compared to the normal programmed dosage regimen for the patient using a fast-acting insulin composition that does not contain a hyaluronan-degrading enzyme. In particular examples, the basal rate and/or bolus dose of insulin is increased 1% to 50%, 5% to 40%, 10% to 20% or 5% to 15% compared to the normal programmed dosage regimen for the patient using a fast-acting insulin composition that does not contain a hyaluronan-degrading enzyme. By practice of the method, the total insulin action is increased compared to the dosage regimen that does not include a systematic increase in insulin delivery over the course of infusion set. For example, the total insulin action as measured by a cumulative glucose infusion (U/kg) in a euglycemic clamp experiment, can increase by at least or about or 1.1-fold, 1.2 fold, 1.3-fold, 1.4-fold, 1.5-fold, 1.6-fold, 1.7-fold, 1.8-fold, 1.9-fold, 2.0-fold, 3.0-fold, 4.0-fold, 5.0-fold or more.

In some examples, the basal insulin and/or bolus insulin are increased at least once per day during the course of the infusion set life. The bolus insulin that can be increased includes the prandial dose for a given mean and/or the correction bolus for a given hyperglycemic correction and bolus on board quantity of insulin.

In further examples, prior to performing CSII with a super-fast acting insulin, a hyaluronan-degrading enzyme is administered to the patient immediately before or immediately after initiation of infusion of the CSII as described in C.1.a above. Hyaluronan-degrading enzymes are well known to one of skill in the art, and are described in Section E below. Any such hyaluronan-degrading enzymes can be employed in practice of the method by administering immediately prior to or immediately after initiation of a CSII of a super-fast acting insulin composition in the method herein.

2. Insulin Pumps and Other Insulin Delivery Devices

An insulin delivery device used in the methods herein includes an insulin pump or other similar device capable of continuous subcutaneous insulin infusion. Insulin delivery devices, including open loop and closed loop systems, typically contain at least one disposable reservoir containing an insulin formulation, a pump (including any controls, software, processing modules and/or batteries) and a disposable infusion set, including a cannula or needle for subcutaneous injection and a tube connecting the cannula or needle to the insulin reservoir. Closed loop delivery devices additionally include a glucose monitor or sensor. For use in the methods herein, the insulin delivery device can contain a reservoir containing either a fast-acting insulin or a super-fast acting insulin co-formulation of insulin and a hyaluronan degrading enzyme.

The insulin or super-fast acting co-formulations can be administered continuously and/or in bolus injections. Users set the pump to give a steady trickle or “basal” amount of insulin formulation continuously throughout the day. Pumps also release additional (“bolus”) doses of insulin formulation at meals and at times when blood sugar is too high based on user input. Frequent blood glucose monitoring is essential to determine insulin dosages and to ensure that insulin is delivered appropriately. This can be achieved by manual monitoring, a separate or contained glucose monitor. Further, an insulin delivery device user has the ability to influence the profile of the insulin by shaping the bolus. For example, a standard bolus can be administered, which is an infusion similar to a discrete injection in that all of the dose is pumped immediately. An extended bolus is a slow infusion over time that avoids a high initial dose and extends the action of the composition. A combination bolus containing both a standard bolus and an extended bolus also can be administered using an insulin pump or other continuous delivery system.

Insulin delivery devices are known in the art and described elsewhere, including, but not limited to, in U.S. Pat. Nos. 6,554,798, 6,641,533, 6,744,350, 6,852,104, 6,872,200, 6,936,029, 6,979,326, 6,999,854, 7,025,713 and 7,109,878. Insulin delivery devices also can be connected to a glucose monitor or sensor, e.g., a closed-loop system, and/or can contain a means to calculate the recommended insulin dose based upon blood glucose levels, carbohydrate content of a meal, or other input. Further insulin delivery devices can be implantable or can be external to the subject. The use of external insulin infusion pumps requires careful selection of individuals, meticulous monitoring, and thorough education and long term ongoing follow-up. This care is generally provided by a multidisciplinary team of health professionals with specific expertise and experience in the management of individuals on insulin pump treatment.

a. Open Loop Systems

Open loop systems can be used with the co-formulations provided herein. Open loop systems typically contain at least one disposable reservoir containing an insulin formulation, a pump (including any controls, software, processing modules and/or batteries) and a disposable infusion set, including a cannula or needle for subcutaneous injection and a tube connecting the cannula or needle to the insulin reservoir. The open loop system infuses in small (basal) doses every few minutes and large (bolus) doses that the patient sets manually. But, an open loop system does not contain a glucose monitor or sensor and therefore cannot respond to changes in the patient's serum glucose levels. Various methods and devices used to measure blood glucose levels are known to one of skill in the art. The conventional technique used by many diabetics for personally monitoring their blood glucose level includes the periodic drawing of blood, the application of that blood to a test strip, and the determination of the blood glucose level using calorimetric, electrochemical, or photometric detection. A variety of devices have been developed for continuous or automatic monitoring of analytes, such as glucose, in the blood stream or interstitial fluid. Some of these devices use electrochemical sensors which are directly implanted into a blood vessel or in the subcutaneous tissue of a patient. Exemplary methods and devices for monitoring glucose levels include, but are not limited to, those described in U.S. Pat. Nos. 5,001,054, 5,009,230, 5,713,353, 6,560,417, 6,574,490, 6,892,085, 6,958,809, 7,299,081, 7,774,145, 7,826,879, 7,857,760 and 7,885,699, which are incorporated herein by reference.

Insulin delivery systems, such as insulin pumps, are known in the art and can be used in the open loop systems. Exemplary open loop insulin delivery devices (such as those described above) include, but are not limited to, those described in U.S. Pat. Nos. 4,562,751, 4,678,408, 4,685,903, 4,373,527, 4,573,994, 6,554,798, 6,641,533, 6,744,350, 6,852,104, 6,872,200, 6,936,029, 6,979,326, 6,999,854, 7,109,878, 7,938,797 and 7,959,598, which are incorporated by reference herein. These and similar systems, easily identifiable by one of skill in the art, can be used to deliver the co-formulations provided herein. The insulin delivery devices typically contain one or more reservoirs, which generally are disposable, containing an insulin preparation, such as a co-formulation of a fast acting insulin and hyaluronan degrading enzyme described herein. In some examples, the co-formulations are delivered using an infusion tube and a cannula or needle. In other examples, the infusion device is attached directly to the skin and the co-formulations flow from the infusion device, through a cannula or needle directly into the body without the use of a tube. In further examples, the infusion device is internal to the body and an infusion tube optionally can be used to deliver the co-formulations.

b. Closed Loop Systems

Closed loop systems, sometimes referred to as an artificial pancreas, are of particular interest for use with the co-formulations provided herein. Closed loop systems refer to systems with an integrated continuous glucose monitor, an insulin pump or other delivery system and controller that includes a mathematical algorithm that constantly calculates the required insulin infusion for glycemic control based upon real time measurements of blood glucose levels. Such systems, when optimized, can facilitate constant and very tight glycemic control, similar to the natural insulin response and glycemic control observed in a healthy non-diabetic subject. To be effective, however, closed loop systems require both a reliable and accurate continuous glucose monitor, and delivery of an insulin with a very fast action. For example, delays in insulin absorption and action associated with subcutaneous delivery of fast-acting insulins can lead to large postprandial glycemic excursions (Hovorka et al. (2006) Diabetic Med. 23:1-12). The delay because of insulin absorption, insulin action, interstitial glucose kinetics, and the transport time for ex vivo-based monitoring systems, such as those based on the microdialysis technique, can result in an overall 100 minute or more time lag from the time of insulin delivery to the peak of its detectable glucose-lowering effect (Hovorka et al. (2006) Diabetic Med. 23:1-12). Thus, once administered, insulin will continue to increase its measurable effect for nearly 2 hours. This can complicate effective lowering of glucose concentration following meal ingestion using a closed-loop system. First, a glucose increase has to be detected. However, this typically happens only after an approximate 10-40 minute lag. The system must determine that a meal has been digested and administer an appropriate insulin dose. The ability of the system to compensate subsequently for a ‘misjudged’ insulin dose is compromised by long delays and the inability to ‘withdraw’ insulin once administered. Such problems can, at least in part, be overcome by using the co-formulations of a fast-acting insulin and hyaluronan degrading enzyme, such as those provided herein, which can exhibit an increased rate and level of absorption and an associated improvement in the pharmacodynamics (see e.g. U.S. Publication No. US20090304665 and International PCT Publication No. WO2009134380). Co-formulations of fast-acting insulin and a hyaluronan degrading enzyme have a reduced tₘₐₓ (i.e. achieve maximal concentration faster) than fast-acting insulins alone and begin controlling blood glucose levels faster than fast-acting insulins alone. This increased rate of absorbance and onset of action reduces the lag between insulin action and glucose monitoring and input, resulting in a more effective closed loop system that can more tightly control blood glucose levels, reducing glycemic excursions.

Closed loop systems are well known in the art and have been described elsewhere, including, but not limited to, U.S. Pat. Nos. 5,279,543, 5,569,186, 6,558,351, 6,558,345, 6,589,229, 6,669,663, 6,740,072, 7,267,665, 7,354,420 and 7,850,674, which are incorporated by reference herein. These and similar systems, easily identifiable by one of skill in the art, can be used to deliver the co-formulations provided herein. Closed loops systems include a sensor system to measure blood glucose levels, a controller and a delivery system. This integrated system is designed to model a pancreatic beta cell (β-cell), such that it controls an infusion device to deliver insulin into a subject in a similar concentration profile as would be created by fully functioning human (β-cells when responding to changes in blood glucose concentrations in the body. Thus, the system simulates the body's natural insulin response to blood glucose levels and not only makes efficient use of insulin, but also accounts for other bodily functions as well since insulin has both metabolic and mitogenic effects. Further, the glycemic control achieved using a closed loop system is achieved without requiring any information about the size and timing of a meal, or other factors. The system can rely solely on real time blood glucose measurements. The glucose sensor generates a sensor signal representative of blood glucose levels in the body, and provides the sensor signal to the controller. The controller receives the sensor signal and generates commands that are communicated to the insulin delivery system. The insulin delivery system receives the commands and infuses insulin into the body in response to the commands.

Provided below are descriptions of exemplary components of closed loop systems that can be used to deliver the co-formulations of a fast acting insulin and a hyaluronan degrading enzyme provided herein. It is understood that one of skill in the art can readily identify suitable closed loop systems for use with the co-formulations. Such systems have been described in the art, including but not limited to, those described in U.S. Pat. Nos. 5,279,543, 5,569,186, 6,558,351, 6,558,345, 6,589,229, 6,669,663, 6,740,072, 7,267,665 and 7,354,420. The individual components of the systems also have been described in the art, individually and in the context of a closed loops system for use in achieving glycemic control. It is understood that the examples provided herein are exemplary only, and that other closed loop systems or individual components can be used to deliver the co-formulations provided herein.

Closed loop systems contain a glucose sensor or monitor that functions continuously. Such devices can contain needle-type sensors that are inserted under the skin and attached to a small transmitter that communicates glucose data wirelessly by radiofrequency telemetry to a small receiver. In some examples, the sensor is inserted through the subject's skin using an insertion needle, which is removed and disposed of once the sensor is positioned in the subcutaneous tissue. The insertion needle has a sharpened tip and an open slot to hold the sensor during insertion into the skin (see e.g. U.S. Pat. Nos. 5,586,553 and 5,954,643). The sensor used in the closed loop system can optionally contain three electrodes that are exposed to the interstitial fluid (ISF) in the subcutaneous tissue. The three electrodes include a working electrode, a reference electrode and a counter electrode that are used to form a circuit. When an appropriate voltage is supplied across the working electrode and the reference electrode, the ISF provides impedance between the electrodes. An analog current signal flows from the working electrode through the body and to the counter electrode. The voltage at the working electrode is generally held to ground, and the voltage at the reference electrode can be held at a set voltage Vset, such as, for example, between 300 and 700 mV. The most prominent reaction stimulated by the voltage difference between the electrodes is the reduction of glucose as it first reacts with the glucose oxidase enzyme (GOX) to generate gluconic acid and hydrogen peroxide (H₂O₂). Then the H₂O₂ is reduced to water (H₂O) and (O⁻) at the surface of the working electrode. The O⁻ draws a positive charge from the sensor electrical components, thus repelling an electron and causing an electrical current flow. This results in the analog current signal being proportional to the concentration of glucose in the ISF that is in contact with the sensor electrodes (see e.g. U.S. Pat. No. 7,354,420).

In some examples, more than one sensor is used to measure blood glucose. For example, redundant sensors can be used and the subject can be notified when a sensor fails by the telemetered characteristic monitor transmitter electronics. An indicator also can inform the subject of which sensors are still functioning and/or the number of sensors still functioning. In other examples, sensor signals are combined through averaging or other means. Further, different types of sensors can be used. For example, an internal glucose sensor and an external glucose sensor can be used to measure blood glucose at the same time.

Glucose sensors that can be used in a closed loop system are well known and can be readily identified and, optionally, further modified, by one of skill in the art. Exemplary internal glucose sensors include, but are not limited to, those described in U.S. Pat. Nos. 5,497,772, 5,660,163, 5,791,344, 5,569,186, 6,895,265 and 7,949,938. Exemplary of a glucose sensor that uses fluorescence is that described in U.S. Pat. No. 6,011,984. Glucose sensor systems also can use other sensing technologies, including light beams, conductivity, jet sampling, micro dialysis, microporation, ultra sonic sampling, reverse iontophoresis, or other methods (e.g. U.S. Pat. Nos. 5,433,197 and 5,945,676, and International Pat. Pub. WO 199929230). In some examples, only the working electrode is located in the subcutaneous tissue and in contact with the ISF, and the counter and reference electrodes are located external to the body and in contact with the skin. The counter electrode and the reference electrode can be located on the surface of a monitor housing and can be held to the skin as part of a telemetered characteristic monitor. In further examples, the counter electrode and the reference electrode are held to the skin using other devices, such as running a wire to the electrodes and taping the electrodes to the skin, incorporating the electrodes on the underside of a watch touching the skin. Still further, more than one working electrode can be placed into the subcutaneous tissue for redundancy. Interstitial fluid also can be harvested from the body of a subject and flowed over an external sensor that is not implanted in the body.

The controller receives input from the glucose sensor. The controller is designed to model a pancreatic beta cell (β-cell) and provide commands to the insulin delivery device to infuse the required amount of insulin for glycemic control. The controller utilizes software with algorithms to calculate the required amount of insulin based upon the glucose levels detected by the glucose sensor. Exemplary algorithms include those that model the β-cells closely, since algorithms that are designed to minimize glucose excursions in the body, without regard for how much insulin is delivered, can cause excessive weight gain, hypertension, and atherosclerosis. Typically, the system is intended to emulate the in vivo insulin secretion pattern and to adjust this pattern consistent with the in vivo β-cell adaptation experienced by normal healthy individuals. Control algorithms useful for closed loop systems include those utilized by a proportional-integral-derivative (PID) controller. Proportional derivative controllers and model predictive control (MPC) algorithms also can be used in some systems (Hovorka et al. (2006) Diabetic Med. 23:1-12). Exemplary algorithms include, but are not limited to, those described Hovorka et al. (Diabetic Med. (2006) 23:1-12), Shimoda et al., (Front Med Biol Eng (1997) 8:197-211), Shichiri et al. (Artif: Organs (1998) 22:32-42), Steil et al. (Diabetes Technol Ther (2003) δ: 953-964), Kaletz et al., (Acta Diabetol. (1999) 36:215) and U.S. Pat. Nos. 5,279,543, 5,569,186, 6,558,351, 6,558,345, 6,589,229, 6,740,042, 6,669,663, 6,740,072, 7,267,665 and 7,354,420 and U.S. Pat. Pub. No. 20070243567.

In one example, a PID controller is utilized in the closed loop system. A PID controller continuously adjusts the insulin infusion by assessing glucose excursions from three viewpoints: the departure from the target glucose (the proportional 1.5 component), the area under the curve between ambient and target glucose (the integral component), and the change in ambient glucose (the derivative component). Generally, the in vivo β-cell response to changes in glucose is characterized by “first” and “second” phase insulin responses. The biphasic insulin response of a β-cell can be modeled using components of a proportional, plus integral, plus derivative (PID) controller (see e.g. U.S. Pat. No. 7,354,420).

The controller generates commands for the desired insulin delivery. Insulin delivery systems, such as insulin pumps, are known in the art and can be used in the closed loop systems. Exemplary insulin delivery devices (such as those described above) include, but are not limited to, those described in U.S. Pat. Nos. 4,562,751, 4,678,408, 4,685,903, 4,373,527, 4,573,994, 6,554,798, 6,641,533, 6,744,350, 6,852,104, 6,872,200, 6,936,029, 6,979,326, 6,999,854 and 7,109,878. The insulin delivery devices typically contain one or more reservoirs, which generally are disposable, containing an insulin preparation, such as a co-formulation of a fast acting insulin and hyaluronan degrading enzyme described herein. In some examples, the co-formulations are delivered using an infusion tube and a cannula or needle. In other examples, the infusion device is attached directly to the skin and the co-formulations flow from the infusion device, through a cannula or needle directly into the body without the use of a tube. In further examples, the infusion device is internal to the body and an infusion tube optionally can be used to deliver the co-formulations. Closed loop systems also can contain additional components, including, but not limited to, filters, calibrators and transmitters.

c. Exemplary Devices

External insulin pump technology includes simple battery powered pumps as well as pumps capable of wireless connectivity to separate parts of the pump device or to other types of devices.

One such pump, the Insulet OmniPod®, involves two separate devices with wireless radiofrequency connection. The first part of this device, referred to as the “Pod”, is a disposable self-adhesive unit that incorporates an insulin reservoir, a microcomputer controlled insulin pump, and a cannulation device. The “Pod” portion of the device is filled with insulin by the individual and then adhered to the skin with an automated cannula inserter. The “Pod” is worn for up to 72 hours and then replaced. The second portion of the device, referred to as the “PDM”, or “Personal Diabetes Manager”, is a hand-held control unit which communicates wirelessly with the “Pod” to control basal-rate and bolus insulin administration. This PDM also contains a blood glucose monitor (not a continuous interstitial monitor) which is integrated into the control system of the Pod, allowing individuals to use this data in dosage calculations. The PDM incorporates a FreeStyle™ blood glucose meter which works similarly to a stand alone blood glucose monitor, requiring the traditional finger-stick method of blood sample acquisition. Once the “Pod” is activated and programmed, it is not necessary for the PDM to remain with the individual until it is used again to check blood glucose levels, give bolus dosages or adjust the basal infusion rate.

Another type of wireless insulin pump device involves the connection between an external insulin pump and a continuous glucose sensor/transmitter. One such device is the Medtronic MiniMed Paradigm REAL-Time System, which incorporates the MiniMed paradigm model insulin pump (models 522, 722 and newer) with the MiniMed continuous glucose sensor and MiniLink™ REAL-Time Transmitter. With this system, the continuous glucose sensor-transmitter wirelessly transmits interstitial glucose concentration data (288 readings in a 24-hour period) to the pump unit, which displays it in “real time”. However, the data transmitted via the wireless feed cannot be seamlessly used for dosage calculations. Such calculations require blood glucose measurements. A glucose sensor/transmitter device may also be wirelessly integrated with an externally worn continuous glucose receiver/monitor (e.g., Guardian® REAL-Time Continuous Glucose Monitoring System).

### D. INSULIN POLYPEPTIDES

The CSII methods provided herein use a fast-acting insulin formulation or a fast-acting insulin and PH20 combination or co-formulation (i.e. a super-fast acting insulin composition as described in Section F). Fast-acting insulins include a regular insulin or an insulin analog (e.g. called a fast-acting analog or a rapid-acting analog, used interchangeably herein) that is modified (e.g. by amino acid replacement) to reduce self-association of insulin and result in more rapid dissociation of hexamers.

Insulin is a polypeptide composed of 51 amino acid residues that is 5808 daltons in molecular weight. It is produced in the beta-cell islets of Langerhans in the pancreas. An exemplary human insulin is translated as a 110 amino acid precursor polypeptide, preproinsulin (SEQ ID NO:101), containing a 24 amino acid signal peptide to ER, the signal sequence is cleaved, resulting in proinsulin (SEQ ID NO:102). The proinsulin molecule is subsequently converted into a mature insulin by actions of proteolytic enzymes, known as prohormone convertases (PC1 and PC2) and by actions of the exoprotease carboxypeptidase E. This results in removal of 4 basic amino acid residues and the remaining 31 amino acid C-peptide or connecting chain (corresponding to amino acid residues 57 to 87 of the preproinsulin polypeptide set forth in SEQ ID NO:101) The resulting insulin contains a 21 amino acid A-chain (corresponding to amino acid residues 66 to 86 of the proinsulin polypeptide set forth in SEQ ID NO:102) and a 30 amino acid B-chain (corresponding to amino acid residues 1 to 30 of the proinsulin polypeptide set forth in SEQ ID NO:102), which are cross-linked by disulfide bonds. Typically, mature insulin contains three disulfide bridges: one between position 7 of the A-chain and position 7 of the B-chain, a second between position 20 of the A-chain and position 19 of the B-chain, and a third between positions 6 and 11 of the A-chain. The sequence of the A chain of a mature insulin is set forth in SEQ ID NO:103 and the sequence of the B-chain is set forth in SEQ ID NO:104.

Reference to insulin includes preproinsulin, proinsulin and insulin polypeptides in single-chain or two-chain forms, truncated forms thereof that have activity, and includes allelic and species variants, variants encoded by splice variants and other variants, such as insulin analogs or other derivatized forms, including polypeptides that have at least 40%, 45%, 50%, 55%, 60%, 65%, 70%, 75%, 80%, 85%, 90%, 95%, 96%, 97%, 98%, 99% or more sequence identity to the precursor polypeptide set forth in SEQ ID NO:101 or the mature form thereof, so long as the insulin binds to the human insulin receptor to initiate a signaling cascade that results in an increase of glucose uptake and storage and/or a decrease of endogenous glucose production. For example, insulins include species variants of insulin. These include, but are not limited to, insulins derived from bovine (set forth in SEQ ID NO:133) and porcine (SEQ ID NO:123). Bovine insulin differs from human insulin at amino acids 8 and 10 of the A chain, and amino acid 30 of the B chain. Porcine insulin only differs from human insulin at amino acid 30 in the B chain where, like the bovine sequence, there is an alanine substitution in place of threonine. Other exemplary species variants of insulin are set forth in any of SEQ ID NOS: 105-146.

Also included among variants of insulin are insulin analogs that contain one or more amino acid modifications compared to a human insulin set forth in SEQ ID NO: 103 and 104 (A and B chains). These variants include fast-acting or longer-acting insulin analogs (all designated herein as a fast-acting insulin analog, although it is understood that for purposes herein this includes rapid-acting and longer-acting insulin analog forms). Exemplary insulin analogs (A and/or B chains), including fast-acting and longer-acting analog forms, are set forth in SEQ ID NOS:147-165, 182-184). For example, insulin analogs include, but are not limited to, glulisine (LysB3, GIuB29; set forth in SEQ ID NO:103 (A-chain) and SEQ ID NO:149 (B-chain)), HMR-1 153 (LysB3, IleB28; set forth in SEQ ID NO:103 (A-chain) and SEQ ID NO:182 (B-chain)), HMR-1423 (GlyA21, HisB31, HisB32; set forth in SEQ ID NO:183 (A-chain) and SEQ ID NO:184 (B-chain)), insulin aspart (AspB28; set forth in SEQ ID NO:103 (A-chain) and SEQ ID NO:147 (B-chain)), and insulin lispro (LysB28, ProB29; set forth in SEQ ID NO:103 (A-chain) and SEQ ID NO:148 (B-chain)). In every instance above, the nomenclature of the analogs is based on a description of the amino acid substitution at specific positions on the A or B chain of insulin, numbered from the N-terminus of the chain, in which the remainder of the sequence is that of natural human insulin.

Hence, regular insulin used in the infusion methods herein is a mature insulin that contains a sequence of amino acids set forth in SEQ ID NOS: 103 and 104. Exemplary of a regular human insulin is recombinant human insulin designated Humulin® R. Regular insulins also includes species variants of mature insulin having an A and B chain, for example, mature forms of any of SEQ ID NOS: 105-146. Other exemplary insulin analogs included in the co-formulations herein include, but are not limited to an insulin that has a sequence of amino acids set forth in SEQ ID NO:103 (A-chain) and SEQ ID NO:149 (B-chain); a sequence of amino acids set forth in SEQ ID NO:103 (A-chain) and SEQ ID NO:147 (B-chain); or a sequence of amino acids set forth in SEQ ID NO:103 (A-chain) and SEQ ID NO:148 (B-chain).

Any of the above insulin polypeptides include those that are produced by the pancreas from any species, such as a human, and also include insulins that are produced synthetically or using recombinant techniques. For example, as described elsewhere herein, insulin can be produced biosynthetically by expressing synthetic genes for A and B chains of insulin, by expressing the entire proinsulin and exposing it to the appropriate enzymatic and chemical methods to generate a mature insulin, or by expressing A and B chains connected by a linker peptide (see e.g., DeFelippis et al. (2002) Insulin Chemistry and Pharmacokinetics. In Ellenberg and Rifkin's Diabetes Mellitus (pp. 481-500) McGraw-Hill Professional).

Insulins also include monomeric and oligomeric forms, such as hexameric forms. Insulin can exist as a monomer as it circulates in the plasma, and it also binds to its receptor while in a monomeric form. Insulin, however, has a propensity to self-associate into dimers, and in the presence of metal ions such as Zn²⁺ can readily associate into higher order structures such as hexamers. There are two symmetrical high affinity binding sites for Zn²⁺, although other weaker zinc-binding sites also have been reported (see e.g., DeFelippis et al. (2002) Insulin Chemistry and Pharmacokinetics. In Ellenberg and Rifkin's Diabetes Mellitus (pp. 481-500) McGraw-Hill Professional). Self-association is important for the stability of the molecule to prevent chemical degradation and physical denaturation. Thus, in storage vesicles in pancreatic beta-cells, insulin exists as a hexamer. Upon release into the extracellular space, however, it is believed that the insulin hexamers can experience a change in pH to more neutral conditions and the zinc ion-containing hexamers are diluted, which destabilizes the hexamer. There may be other reasons contributing to the destabilization of the insulin hexamer in the extracellular space. Insulin is thus predominantly found in the blood as a monomer. To take advantage of the stabilizing effects, most commercial formulations of insulin contain zinc ions in sufficient amounts to promote self-association into hexamers. The hexameric structure, however, slows down the absorption rate of these formulations upon subcutaneous administration.

Insulin is used as a therapeutic for glycemic control, such as in diabetic patients. There are various types of insulin formulations that exist, depending on whether the insulin is being administered to control glucose for basal therapy, for prandial therapy, or for a combination thereof. Insulin formulations can be provided solely as fast-acting formulations, solely as basal-acting formulations (i.e., intermediate-acting and/or long-acting forms), or as mixtures thereof (see e.g., Table 2). Typically, mixtures contain a fast-acting and an intermediate- or long-acting insulin. For example, fast-acting insulins can be combined with an NPH insulin (an exemplary intermediate-acting insulin as discussed below) in various mixture ratios including 10:90, 20:80, 30:70, 40:60, and 50:50. Such premixed preparations can reduce the number of daily insulin injections by conveniently providing both meal-related and basal insulin requirements in a single formulation.

Preparations of insulin include an insulin polypeptide or variant (i.e. analog) thereof formulated in a specific manner. In some instances, it is the components and substances in the formulation that impart different properties on the insulin, such as different duration of action. For example, most insulin preparations contain a metal ion, such as zinc, in the formulation, which stabilizes the insulin by promoting self-association of the molecule. Self-association into hexameric forms can affect the absorption of insulin upon administration. Further, some longer-acting basal insulin formulations are prepared by precipitating insulin from an acetate buffer (instead of phosphate) by the addition of zinc. Large crystals of insulin with high zinc content, when collected and resuspended in a solution of sodium acetate-sodium chloride (pH 7.2 to 7.5), are slowly absorbed after subcutaneous injection and exert an action of long duration. This crystal preparation is named extended insulin zinc suspension (ultralente insulin). Other zinc-containing insulin preparations include, for example, semilente insulins (prompt insulin zinc suspensions) and lente insulins (insulin zinc suspensions), which differ predominantly in the zinc concentration used. Zinc-containing insulin preparations also include those that are modified by protamine, such as NPH insulin.

In another example, a precipitation agent, such as protamine, can be added to an insulin polypeptide to generate a microcrystalline suspension. Typically, crystalline insulins have a prolonged duration of action compared to insulins that do not exist in crystalline form. A protamine zinc insulin, when injected subcutaneously in an aqueous suspension, dissolves only slowly at the site of deposition, and the insulin is absorbed at a retarded rate. Protamine zinc suspension insulin has largely been replaced by isophane insulin suspension, also known as NPH insulin. It is a modified protamine zinc insulin suspension that is crystalline. The concentrations of insulin, protamine, and zinc are so arranged that the preparation has an onset and a duration of action intermediate between those of regular insulin and protamine zinc insulin suspension.

Further, pH differences in the preparations also influence the type and property of insulin. Most insulins are formulated at neutral pH. One exception is insulin glargine, which is provided as a commercial formulation at pH 4.0. By virtue of the addition of two arginines to the C-terminus of the B-chain, the isoelectric point of the glargine insulin is shifted making it more soluble at an acidic pH. An additional amino acid change exists in the A chain (N21G) to prevent deamidation and dimerization resulting from an acid-sensitive asparagine. The sequence of the A chain of glargine insulin is set forth in SEQ ID NO:150 and the B-chain is set forth in SEQ ID NO:151. Since exposure to physiologic pH occurs upon administration, microprecipitates are formed, which make glargine similar to a crystalline, long-acting insulin.

Table 2 below summarizes various types of insulin, their onset of action and their application.

TABLE 2
Types of Insulins

| Type | Brand name | Onset | | Peak | | Duration | | Application |
|---|---|---|---|---|---|---|---|---|
| Fast-acting: | Lispro (e.g. | 5-15 | minutes | 45-90 | minutes | 3-4 | hours | Post-prandial |
| Insulin | Humalog ®); | | | | | | | glucose control |
| analogs | Aspart (e.g., | | | | | | | |
| | NovoLog ®); | | | | | | | |
| | Glulisine | | | | | | | |
| Fast-acting: | Regular Insulin | 30 minutes-1 hour | | 2-5 | hours | 5-8 | hours | Post-prandial |
| Regular | (e.g., Humulin ® | | | | | | | glucose control |
| insulin | R; Novolin ® R; | | | | | | | |
| | Velosulin ® | | | | | | | |
| | Human) | | | | | | | |
| Intermediate- | Lente ® (e.g., | 1-3 | hours | 6-12 | hours | 20-24 | hours | Basal insulin |
| Acting | Humulin ® L, | | | | | | | supplementation |
| | Novolin ® L); | | | | | | | |
| | NPH (e.g., | | | | | | | |
| | Humulin ® N, | | | | | | | |
| | Novolin ® N); | | | | | | | |
| Long- | Ultralente (e.g. | 4-6 | hours | 18-28 | hours | 28 | hours | Basal insulin |
| lasting | Humulin ® U); | | | | | | | supplementation |
| | glargine; detemir | | | | | | | |
| | (an analog) | | | | | | | |

| | | | | |
|---|---|---|---|---|
| Mixtures | Humulin ® 50/50; | Varies | Varies | Varies |
| | Humulin ® 70/30; | | | |
| | Novolin ® 70/30; | | | |
| | Humalog ® Mix 75/25 | | | |

The most commonly used insulins are fast-acting insulins, which include regular insulin (i.e. native or wildtype insulin, including allelic and species variants thereof) and fast-acting insulin analogs. For purposes herein, reference to insulin is a fast-acting insulin, unless specifically noted otherwise.

Fast-Acting Insulins

Fact-acting insulins that can be used in the CSII infusion methods provided herein include regular insulin, which is the wild-type or native insulin, and fast-acting insulin analogs. By virtue of their fast absorption rate compared to basal-acting insulins, fast-acting insulins are used predominantly for post-prandial control purposes. Exemplary fast-acting insulins are set forth in Table 3 below. Fast-acting insulins also include any known in the art, such as, but not limited to, any insulin preparations and devices disclosed in U.S. Pat. No. 7,279,457 and U.S. Pat. Pub. Nos. 20070235365, 20080039368, 20080039365, 20070086952, 20070244467, and 20070191757. Any fast-acting insulin can be prepared as a formulation either alone or in combination or co-formulated with PH20 for use in the CSII methods herein. Such a formulation also can further include a mixture of a fast-acting insulin with an intermediate or long-acting insulin, in addition to a hyaluronan degrading enzyme.

TABLE 3
Fast Acting Insulins

| | | A-chain | B-chain | Commercial |
|---|---|---|---|---|
| Name | Species | (SEQ ID NO) | (SEQ ID NO) | Name |
| Regular | Human | 103 | 104 | Humulin R ®; |
| Insulin | | | | Novolin ® R; |
| | | | | Velosulin ® |
| Regular | Porcine | 88-108 of SEQ | 25-54 of SEQ | Iletin II ®; |
| Insulin | | ID NO: 123 | ID NO: 123 | |
| Insulin | Human | 103 | 147 | Novolog ® |
| Aspart | analog | | | |
| Insulin | Human | 103 | 148 | Humalog ® |
| Lispro | analog | | | |
| Insulin | Human | 103 | 149 | Apidra ® |
| Glulisine | analog | | | |

a. Regular Insulin

Regular insulins include the native or wildtype insulin polypeptide. These include human insulin, as well as insulins from bovine, porcine and other species. Regular human insulins are marketed as Humulin® R, Novolin® R and Velosulin®. Porcine insulin was marketed as Iletin II®. Generally, regular insulin, when administered subcutaneously alone, has an onset of action of 30 minutes. Maximal plasma levels are seen in 1-3 hours and the duration of intensity increases with dosage. The plasma half-life following subcutaneous administration is about 1.5 hours.

b. Fast-Acting Analogs (Also Called Rapid-Acting Insulin)

Fast-Acting insulin analogs, which are often called rapid-acting insulins in the art, are modified forms of insulin that typically contain one or more amino acid changes. The analogs are designed to reduce the self-association of the insulin molecule for the purpose of increasing the absorption rate and onset of action as compared to regular insulin. Generally, such analogs are formulated in the presence of zinc, and thus exist as stable zinc hexamers. Due to the modification, however, they have a quicker dissociation from the hexameric state after subcutaneous administration compared to regular insulin.

i. Insulin Lispro

Human insulin lispro is an insulin polypeptide formulation containing amino acid changes at position 28 and 29 of the B-chain such that the Pro-Lys at this position in wild-type insulin B-chain set forth in SEQ ID NO:104 is inverted to Lys-Pro. The sequence of insulin lispro is set forth in SEQ ID NO:103 (A-chain) and SEQ ID NO: 148 (B-chain). It is marketed under the name Humalog® (insulin lispro, rDNA origin). The result of the inversion of these two amino acids is a polypeptide with a decreased propensity to self-associate, which allows for a more rapid onset of action. Specifically, the sequence inversion in the B-chain results in the elimination of two hydrophobic interactions and weakening of two beta-pleated sheet hydrogen bonds that stabilize the dimer (see e.g., DeFelippis et al. (2002) Insulin Chemistry and Pharmacokinetics. In Ellenberg and Rifkin's Diabetes Mellitus (pp. 481-500) McGraw-Hill Professional). The polypeptide self-associates and forms hexamers as a result of excipients provided in the formulation, such as antimicrobial agents (e.g. m-cresol) and zinc for stabilization. Nevertheless, due to the amino acid modification, insulin lispro is more rapidly acting than regular insulin.

ii. Insulin Aspart

Human insulin aspart is an insulin polypeptide formulation containing an amino acid substitution at position 28 of the B-chain of human insulin set forth in SEQ ID NO:104 from a proline to an aspartic acid. The sequence of insulin aspart is set forth in SEQ ID NO:103 (A-chain) and SEQ ID NO:147 (B-chain). It is marketed under the name Novolog® (insulin aspart [rDNA origin] injection). The modification in insulin aspart confers a negatively-charged side-chain carboxyl group to create charge repulsion and destabilize the monomer-monomer interaction. Further, the removal of the proline eliminates a key hydrophobic interaction between monomers (see e.g., DeFelippis et al. (2002) Insulin Chemistry and Pharmacokinetics. In Ellenberg and Rifkin's Diabetes Mellitus (pp. 481-500) McGraw-Hill Professional). The analog exists largely as a monomer, and is less prone to aggregation compared to other fast-acting analogs such as lispro. Generally, insulin aspart and insulin lispro are similar in their respective pharmacokinetic and pharmacodynamic properties.

iii. Insulin Glulisine

Human insulin glulisine is an insulin polypeptide formulation containing an amino acid substitution in the B-chain at position B3 from asparagine to lysine and at amino acid B29 from lysine to glutamic acid compared to the sequence of the B-chain of human insulin set forth in SEQ ID NO:104. The sequence of insulin glulisine is set forth in SEQ ID NO:103 (A-chain) and SEQ ID NO:149 (B-chain). It is marketed under the name Apidra® (insulin glulisine [rDNA origin] injection). The modifications render the polypeptide molecule less prone to self-association compared to human insulin. Unlike other insulin analogs, the polypeptide is commercially formulated in the absence of the hexamer-promoting zinc (Becker et al. (2008) Clinical Pharmacokinetics, 47:7-20). Hence, insulin glulisine has a more rapid rate of onset than insulin lispro and insulin aspart.

### E. HYALURONAN DEGRADING ENZYMES

Hyaluronan-degrading enzymes, such as a hyaluronidase for example a PH20 (e.g. rHuPH20) can be used in the CSII methods herein. The hyaluronan-degrading enzyme can be formulated separately for use, for example, in leading edge embodiments. In other examples, the hyaluronan-degrading enzyme can be formulated together as a co-formulation with a fast-acting insulin for CSII.

Hyaluronan-degrading enzymes act to degrade hyaluronan by cleaving hyaluronan polymers, which are composed of repeating disaccharides units, D-glucuronic acid (GlcA) and N-acetyl-D-glucosamine (GlcNAc), linked together via alternating β-1→4 and β-1→3 glycosidic bonds. Hyaluronan chains can reach about 25,000 disaccharide repeats or more in length and polymers of hyaluronan can range in size from about 5,000 to 20,000,000 Da in vivo. Hyaluronan, also called hyaluronic acid or hyaluronate, is a non-sulfated glycosaminoglycan that is widely distributed throughout connective, epithelial, and neural tissues. Hyaluronan is an essential component of the extracellular matrix and a major constituent of the interstitial barrier. By catalyzing the hydrolysis of hyaluronan, hyaluronan-degrading enzymes lower the viscosity of hyaluronan, thereby increasing tissue permeability and increasing the absorption rate of fluids administered parenterally. As such, hyaluronan-degrading enzymes, such as hyaluronidases, have been used, for example, as spreading or dispersing agents in conjunction with other agents, drugs and proteins to enhance their dispersion and delivery.

Accordingly, hyaluronan-degrading enzymes include any enzyme having the ability to catalyze the cleavage of a hyaluronan disaccharide chain or polymer. In some examples the degrading enzyme cleaves the β-1→4 glycosidic bond in the hyaluronan chain or polymer. In other examples, the degrading enzyme catalyze the cleavage of the β-1→6 glycosidic bond in the hyaluronan chain or polymer. Exemplary of hyaluronan degrading enzymes in the co-formulations provided herein are hyaluronidases that are secreted into the media when expressed from a cell expression system, including natural hyalurondiases that do not contain a glycosylphosphatidylinositol (GPI) anchor or truncated hyaluronidases that lack one or more amino acids of the GPI anchor or hyaluronidases that are otherwise not associated with the cell membrane when expressed therefrom. Such hyaluronidases can be produced recombinantly or synthetically. Other exemplary hyaluronan degrading enzymes include, but are not limited to particular chondroitinases and lyases that have the ability to cleave hyaluronan.

Hyaluronan-degrading enzymes provided in the methods herein also include allelic or species variants or other variants, of a hyaluronan-degrading enzyme as described herein. For example, hyaluronan-degrading enzymes can contain one or more variations in its primary sequence, such as amino acid substitutions, additions and/or deletions. A variant of a hyaluronan-degrading enzyme generally exhibits at least or about 60%, 70%, 80%, 90%, 91%, 92%, 93%, 94%, 95%, 96%, 97%, 98%, 99% or more sequence identity compared to the hyaluronan-degrading enzyme not containing the variation. Any variation can be included in the hyaluronan degrading enzyme for the purposes herein provided the enzyme retains hyaluronidase activity, such as at least or about 5%, 10%, 15%, 20%, 25%, 30%, 35%, 40%, 45%, 50%, 55%, 60%, 65%, 70%, 75%, 80%, 85%, 90%, 95% or more of the activity of a hyaluronan degrading enzyme not containing the variation (as measured by in vitro and/or in vivo assays well known in the art and described herein).

Various forms of hyaluronan degrading enzymes, including hyaluronidases have been prepared and approved for therapeutic use in subjects, including humans. For example, animal-derived hyaluronidase preparations include Vitrase® (ISTA Pharmaceuticals), a purified ovine testicular hyaluronidase, and Amphadase® (Amphastar Pharmaceuticals), a bovine testicular hyaluronidase. Hylenex® (Baxter) is a human recombinant hyaluronidase produced by genetically engineered Chinese Hamster Ovary (CHO) cells containing nucleic acid encoding a truncated human PH20 polypeptide (designated rHuPH20). It is understand that any hyaluronan-degrading enzyme, such as any hyaluronidase can be included in the stable co-formulations provided herein (see, e.g., U.S. Pat. No. 7,767,429, and U.S. Publication Nos. 20040268425 and 20100143457, which are incorporated by reference in their entirety).

Typically, for use herein, a human hyaluronan degrading enzyme, such as a human PH20 and in particular a C-terminal truncated human PH20 as described herein, is used. Although hyaluronan degrading enzymes, such as PH20, from other animals can be utilized, such preparations are potentially immunogenic, since they are animal proteins. For example, a significant proportion of patients demonstrate prior sensitization secondary to ingested foods, and since these are animal proteins, all patients have a risk of subsequent sensitization. Thus, non-human preparations may not be suitable for chronic use. If non-human preparations are desired, they can be prepared to have reduced immunogenicity. Such modifications are within the level of one of skill in the art and can include, for example, removal and/or replacement of one or more antigenic epitopes on the molecule.

Hyaluronan degrading enzymes, including hyaluronidases (e.g., PH20), used in the co-formulations provided herein can be recombinantly produced or can be purified or partially-purified from natural sources, such as, for example, from testes extracts. Methods for production of recombinant proteins, including recombinant hyaluronan degrading enzymes, are provided elsewhere herein and are well known in the art.

1. Hyaluronidases

Hyaluronidases are members of a large family of hyaluronan degrading enzymes. There are three general classes of hyaluronidases: mammalian-type hyaluronidases, bacterial hyaluronidases and hyaluronidases from leeches, other parasites and crustaceans. Such enzymes can be used in the co-formulations provided herein.

a. Mammalian-Type Hyaluronidases

Mammalian-type hyaluronidases (EC 3.2.1.35) are endo-β-N-acetyl-hexosaminidases that hydrolyze the β-1→4 glycosidic bond of hyaluronan into various oligosaccharide lengths such as tetrasaccharides and hexasaccharides. These enzymes have both hydrolytic and transglycosidase activities, and can degrade hyaluronan and chondroitin sulfates (CS), generally C4-S and C6-S. Hyaluronidases of this type include, but are not limited to, hyaluronidases from cows (bovine) (SEQ ID NOS:10, 11 and 64 and BH55 (U.S. Pat. Nos. 5,747,027 and 5,827,721)), sheep (Ovis aries) (SEQ ID NO: 26, 27, 63 and 65), yellow jacket wasp (SEQ ID NOS:12 and 13), honey bee (SEQ ID NO:14), white-face hornet (SEQ ID NO:15), paper wasp (SEQ ID NO:16), mouse (SEQ ID NOS:17-19, 32), pig (SEQ ID NOS:20-21), rat (SEQ ID NOS:22-24, 31), rabbit (SEQ ID NO:25), orangutan (SEQ ID NO:28), cynomolgus monkey (SEQ ID NO:29), guinea pig (SEQ ID NO:30), chimpanzee (SEQ ID NO:185), rhesus monkey (SEQ ID NO:186) and human hyaluronidases.

Mammalian hyaluronidases can be further subdivided into those that are neutral active, predominantly found in testes extracts, and acid active, predominantly found in organs such as the liver. Exemplary neutral active hyaluronidases include PH20, including but not limited to, PH20 derived from different species such as ovine (SEQ ID NO:27), bovine (SEQ ID NO:11) and human (SEQ ID NO:1). Human PH20 (also known as SPAM1 or sperm surface protein PH20), is generally attached to the plasma membrane via a glycosylphosphatidyl inositol (GPI) anchor. It is naturally involved in sperm-egg adhesion and aids penetration by sperm of the layer of cumulus cells by digesting hyaluronic acid. Exemplary of hyaluronidases used in the co-formulations here are neutral active hyaluronidases.

Besides human PH20 (also termed SPAM1), five hyaluronidase-like genes have been identified in the human genome, HYAL1, HYAL2, HYAL3, HYAL4 and HYALP1. HYALP1 is a pseudogene, and HYAL3 (precursor polypeptide set forth in SEQ ID NO:38) has not been shown to possess enzyme activity toward any known substrates. HYAL4 (precursor polypeptide set forth in SEQ ID NO:39) is a chondroitinase and exhibits little activity towards hyaluronan. HYAL1 (precursor polypeptide set forth in SEQ ID NO:36) is the prototypical acid-active enzyme and PH20 (precursor polypeptide set forth in SEQ ID NO:1) is the prototypical neutral-active enzyme. Acid-active hyaluronidases, such as HYAL1 and HYAL2 (precursor polypeptide set forth in SEQ ID NO:37) generally lack catalytic activity at neutral pH (i.e. pH 7). For example, HYAL1 has little catalytic activity in vitro over pH 4.5 (Frost et al. (1997) Anal. Biochem. 251:263-269). HYAL2 is an acid-active enzyme with a very low specific activity in vitro. The hyaluronidase-like enzymes also can be characterized by those which are generally attached to the plasma membrane via a glycosylphosphatidyl inositol (GPI) anchor such as human HYAL2 and human PH20 (Danilkovitch-Miagkova, et al. (2003) Proc Natl Acad Sci USA 100(8):4580-5), and those which are generally soluble such as human HYAL1 (Frost et al. (1997) Biochem Biophys Res Commun. 236(1):10-5).

PH20

PH20, like other mammalian hyaluronidases, is an endo-β-N-acetyl-hexosaminidase that hydrolyzes the β1→4 glycosidic bond of hyaluronic acid into various oligosaccharide lengths such as tetrasaccharides and hexasaccharides. They have both hydrolytic and transglycosidase activities and can degrade hyaluronic acid and chondroitin sulfates, such as C4-S and C6-S. PH20 is naturally involved in sperm-egg adhesion and aids penetration by sperm of the layer of cumulus cells by digesting hyaluronic acid. PH20 is located on the sperm surface, and in the lysosome-derived acrosome, where it is bound to the inner acrosomal membrane. Plasma membrane PH20 has hyaluronidase activity only at neutral pH, while inner acrosomal membrane PH20 has activity at both neutral and acid pH. In addition to being a hyaluronidase, PH20 also appears to be a receptor for HA-induced cell signaling, and a receptor for the zona pellucida surrounding the oocyte.

Exemplary PH20 proteins include, but are not limited to, human (precursor polypeptide set forth in SEQ ID NO:1, mature polypeptide set forth in SEQ ID NO: 2), bovine (SEQ ID NOS: 11 and 64), rabbit (SEQ ID NO: 25), ovine PH20 (SEQ ID NOS: 27, 63 and 65), cynomolgus monkey (SEQ ID NO: 29), guinea pig (SEQ ID NO: 30), rat (SEQ ID NO: 31), mouse (SEQ ID NO: 32), chimpanzee (SEQ ID NO: 185) and rhesus monkey (SEQ ID NO:186) PH20 polypeptides.

Bovine PH20 is a 553 amino acid precursor polypeptide (SEQ ID NO:11). Alignment of bovine PH20 with the human PH20 shows only weak homology, with multiple gaps existing from amino acid 470 through to the respective carboxy termini due to the absence of a GPI anchor in the bovine polypeptide (see e.g., Frost G I (2007) Expert Opin. Drug. Deliv. 4: 427-440). In fact, clear GPI anchors are not predicted in many other PH20 species besides humans. Thus, PH20 polypeptides produced from ovine and bovine naturally exist as soluble forms. Though bovine PH20 exists very loosely attached to the plasma membrane, it is not anchored via a phospholipase sensitive anchor (Lalancette et al. (2001) Biol Reprod. 65(2):628-36). This unique feature of bovine hyaluronidase has permitted the use of the soluble bovine testes hyaluronidase enzyme as an extract for clinical use (Wydase®, Hyalase®).

The human PH20 mRNA transcript is normally translated to generate a 509 amino acid precursor polypeptide (SEQ ID NO:1) containing a 35 amino acid signal sequence at the N-terminus (amino acid residue positions 1-35) and a 19 amino acid glycosylphosphatidylinositol (GPI) anchor attachment signal sequence at the C-terminus (amino acid residue positions 491-509). The mature PH20 is, therefore, a 474 amino acid polypeptide set forth in SEQ ID NO:2. Following transport of the precursor polypeptide to the ER and removal of the signal peptide, the C-terminal GPI-attachment signal peptide is cleaved to facilitate covalent attachment of a GPI anchor to the newly-formed C-terminal amino acid at the amino acid position corresponding to position 490 of the precursor polypeptide set forth in SEQ ID NO:1. Thus, a 474 amino acid GPI-anchored mature polypeptide with an amino acid sequence set forth in SEQ ID NO:2 is produced.

Although human PH20 is a neutral active hyaluronidase when it exists at the plasma membrane via a GPI anchor, it exhibits activity at both neutral and acidic pH when it is expressed on the inner acrosomal membrane. It appears that PH20 contains two catalytic sites at distinct regions of the polypeptide: the Peptide 1 and Peptide 3 regions (Cherr et al., (2001) Matrix Biology 20:515-525). Evidence suggests that the Peptide 1 region of PH20, which corresponds to amino acid positions 107-137 of the mature polypeptide set forth in SEQ ID NO:2 and positions 142-172 of the precursor polypeptide set forth in SEQ ID NO:1, is required for enzyme activity at neutral pH. Amino acids at positions 111 and 113 (corresponding to the mature PH20 polypeptide set forth in SEQ ID NO:2) within this region appear to be important for activity, as mutagenesis by amino acid replacement results in PH20 polypeptides with 3% hyaluronidase activity or undetectable hyaluronidase activity, respectively, compared to the wild-type PH20 (Arming et al., (1997) Eur. J. Biochem. 247:810-814).

The Peptide 3 region, which corresponds to amino acid positions 242-262 of the mature polypeptide set forth in SEQ ID NO:2, and positions 277-297 of the precursor polypeptide set forth in SEQ ID NO:1, appears to be important for enzyme activity at acidic pH. Within this region, amino acids at positions 249 and 252 of the mature PH20 polypeptide appear to be essential for activity, and mutagenesis of either one results in a polypeptide essentially devoid of activity (Arming et al., (1997) Eur. J. Biochem. 247:810-814).

In addition to the catalytic sites, PH20 also contains a hyaluronan-binding site. Experimental evidence suggest that this site is located in the Peptide 2 region, which corresponds to amino acid positions 205-235 of the precursor polypeptide set forth in SEQ ID NO:1 and positions 170-200 of the mature polypeptide set forth in SEQ ID NO:2. This region is highly conserved among hyaluronidases and is similar to the heparin binding motif. Mutation of the arginine residue at position 176 (corresponding to the mature PH20 polypeptide set forth in SEQ ID NO:2) to a glycine results in a polypeptide with only about 1% of the hyaluronidase activity of the wild type polypeptide (Arming et al., (1997) Eur. J. Biochem. 247:810-814).

There are seven potential N-linked glycosylation sites in human PH20 at N82, N166, N235, N254, N368, N393, N490 of the polypeptide exemplified in SEQ ID NO:1. Because amino acids 36 to 464 of SEQ ID NO:1 appears to contain the minimally active human PH20 hyaluronidase domain, the N-linked glycosylation site N-490 is not required for proper hyaluronidase activity. There are six disulfide bonds in human PH20. Two disulfide bonds between the cysteine residues C60 and C351 and between C224 and C238 of the polypeptide exemplified in SEQ ID NO:1 (corresponding to residues C25 and C316, and C189 and C203 of the mature polypeptide set forth in SEQ ID NO:2, respectively). A further four disulfide bonds are formed between the cysteine residues C376 and C387; between C381 and C435; between C437 and C443; and between C458 and C464 of the polypeptide exemplified in SEQ ID NO:1 (corresponding to residues C341 and C352; between C346 and C400; between C402 and C408; and between C423 and C429 of the mature polypeptide set forth in SEQ ID NO:2, respectively).

b. Bacterial Hyaluronidases

Bacterial hyaluronidases (EC 4.2.2.1 or EC 4.2.99.1) degrade hyaluronan and, to various extents, chondroitin sulfates and dermatan sulfates. Hyaluronan lyases isolated from bacteria differ from hyaluronidases (from other sources, e.g., hyaluronoglucosaminidases, EC 3.2.1.35) by their mode of action. They are endo-β-N-acetylhexosaminidases that catalyze an elimination reaction, rather than hydrolysis, of the β1→4-glycosidic linkage between N-acetyl-beta-D-glucosamine and D-glucuronic acid residues in hyaluronan, yielding 3-(4-deoxy-β-D-gluc-4-enuronosyl)-N-acetyl-D-glucosamine tetra- and hexasaccharides, and disaccharide end products. The reaction results in the formation of oligosaccharides with unsaturated hexuronic acid residues at their nonreducing ends.

Exemplary hyaluronidases from bacteria for co-formulations provided herein include, but are not limited to, hyaluronan degrading enzymes in microorganisms, including strains of Arthrobacter, Bdellovibrio, Clostridium, Micrococcus, Streptococcus, Peptococcus, Propionibacterium, Bacteroides, and Streptomyces. Particular examples of such enzymes include, but are not limited to Arthrobacter sp. (strain FB24) (SEQ ID NO:67), Bdellovibrio bacteriovorus (SEQ ID NO:68), Propionibacterium acnes (SEQ ID NO:69), Streptococcus agalactiae ((SEQ ID NO:70); 18RS21 (SEQ ID NO:71); serotype Ia (SEQ ID NO:72); serotype III (SEQ ID NO:73), Staphylococcus aureus (strain COL) (SEQ ID NO:74); strain MRSA252 (SEQ ID NOS:75 and 76); strain MSSA476 (SEQ ID NO:77); strain NCTC 8325 (SEQ ID NO:78); strain bovine RF122 (SEQ ID NOS:79 and 80); strain USA300 (SEQ ID NO:81), Streptococcus pneumoniae ((SEQ ID NO:82); strain ATCC BAA-255/R6 (SEQ ID NO:83); serotype 2, strain D39/NCTC 7466 (SEQ ID NO:84), Streptococcus pyogenes (serotype M1) (SEQ ID NO:85); serotype M2, strain MGAS10270 (SEQ ID NO:86); serotype M4, strain MGAS10750 (SEQ ID NO:87); serotype M6 (SEQ ID NO:88); serotype M12, strain MGAS2096 (SEQ ID NOS:89 and 90); serotype M12, strain MGAS9429 (SEQ ID NO:91); serotype M28 (SEQ ID NO:92); Streptococcus suis (SEQ ID NOS:93-95); Vibrio fischeri (strain ATCC 700601/ES114 (SEQ ID NO:96)), and the Streptomyces hyaluronolyticus hyaluronidase enzyme, which is specific for hyaluronic acid and does not cleave chondroitin or chondroitin sulfate (Ohya, T. and Kaneko, Y. (1970) Biochim. Biophys. Acta 198:607).

c. Hyaluronidases from Leeches, Other Parasites and Crustaceans

Hyaluronidases from leeches, other parasites, and crustaceans (EC 3.2.1.36) are endo-β-glucuronidases that generate tetra- and hexasaccharide end-products. These enzymes catalyze hydrolysis of 1→3-linkages between β-D-glucuronate and N-acetyl-D-glucosamine residues in hyaluronate. Exemplary hyaluronidases from leeches include, but are not limited to, hyaluronidase from Hirudimidae (e.g., Hirudo medicinalis), Erpobdellidae (e.g., Nephelopsis obscura and Erpobdella punctata,), Glossiphoniidae (e.g., Desserobdella picta, Helobdella stagnalis, Glossiphonia complanata, Placobdella ornata and Theromyzon sp.) and Haemopidae (Haemopis marmorata) (Hovingh et al. (1999) Comp Biochem Physiol B Biochem Mol. Biol. 124(3):319-26). An exemplary hyaluronidase from bacteria that has the same mechanism of action as the leech hyaluronidase is that from the cyanobacteria, Synechococcus sp. (strain RCC307, SEQ ID NO:97).

2. Other Hyaluronan Degrading Enzymes

In addition to the hyaluronidase family, other hyaluronan degrading enzymes can be used in the CSII methods provided herein. For example, enzymes, including particular chondroitinases and lyases, that have the ability to cleave hyaluronan can be employed. Exemplary chondroitinases that can degrade hyaluronan include, but are not limited to, chondroitin ABC lyase (also known as chondroitinase ABC), chondroitin AC lyase (also known as chondroitin sulfate lyase or chondroitin sulfate eliminase) and chondroitin C lyase. Methods for production and purification of such enzymes for use in the compositions, combinations, and methods provided are known in the art (e.g., U.S. Pat. No. 6,054,569; Yamagata, et al. (1968) J. Biol. Chem. 243(7):1523-1535; Yang et al. (1985) J. Biol. Chem. 160(30):1849-1857).

Chondroitin ABC lyase contains two enzymes, chondroitin-sulfate-ABC endolyase (EC 4.2.2.20) and chondroitin-sulfate-ABC exolyase (EC 4.2.2.21) (Hamai et al. (1997) J Biol. Chem. 272(14):9123-30), which degrade a variety of glycosaminoglycans of the chondroitin-sulfate- and dermatan-sulfate type. Chondroitin sulfate, chondroitin-sulfate proteoglycan and dermatan sulfate are the preferred substrates for chondroitin-sulfate-ABC endolyase, but the enzyme also can act on hyaluronan at a lower rate. Chondroitin-sulfate-ABC endolyase degrades a variety of glycosaminoglycans of the chondroitin-sulfate- and dermatan-sulfate type, producing a mixture of Δ4-unsaturated oligosaccharides of different sizes that are ultimately degraded to Δ4-unsaturated tetra- and disaccharides. Chondroitin-sulfate-ABC exolyase has the same substrate specificity but removes disaccharide residues from the non-reducing ends of both polymeric chondroitin sulfates and their oligosaccharide fragments produced by chondroitin-sulfate-ABC endolyase (Hamai, A. et al. (1997). J. Biol. Chem. 272:9123-9130). A exemplary chondroitin-sulfate-ABC endolyases and chondroitin-sulfate-ABC exolyases include, but are not limited to, those from Proteus vulgaris and Flavobacterium heparinum (the Proteus vulgaris chondroitin-sulfate-ABC endolyase is set forth in SEQ ID NO: 98 (Sato et al. (1994) Appl. Microbiol. Biotechnol. 41(1):39-46).

Chondroitin AC lyase (EC 4.2.2.5) is active on chondroitin sulfates A and C, chondroitin and hyaluronic acid, but is not active on dermatan sulfate (chondroitin sulfate B). Exemplary chondroitinase AC enzymes from the bacteria include, but are not limited to, those from Flavobacterium heparinum and Victivallis vadensis, set forth in SEQ ID NOS:99 and 100, respectively, and Arthrobacter aurescens (Tkalec et al. (2000) Applied and Environmental Microbiology 66(1):29-35; Ernst et al. (1995) Critical Reviews in Biochemistry and Molecular Biology 30(5):387-444).

Chondroitinase C cleaves chondroitin sulfate C producing tetrasaccharide plus an unsaturated 6-sulfated disaccharide (delta Di-6S). It also cleaves hyaluronic acid producing unsaturated non-sulfated disaccharide (delta Di-OS). Exemplary chondroitinase C enzymes from the bacteria include, but are not limited to, those from Streptococcus and Flavobacterium (Hibi et al. (1989) FEMS-Microbiol-Lett. 48(2):121-4; Michelacci et al. (1976) J. Biol. Chem. 251:1154-8; Tsuda et al. (1999) Eur. I Biochem. 262:127-133)

3. Truncated Hyaluronan Degrading Enzymes or Other Soluble Forms

Hyaluronan-degrading enzymes can exist in membrane-bound or membrane-associated form, or can be secreted into the media when expressed from cells and thereby exist in soluble form. For purposes herein, hyaluronan degrading enzymes include any hyaluronan degrading enzymes that when expressed and secreted from cells are not associated with the cell membrane, and thereby exist in soluble form. Soluble hyaluronan-degrading enzymes include, but are not limited to hyaluronidases, including non-human hyaluronidases (e.g. animal or bacterial hyaluronidases), such as bovine PH20 or ovine PH20, and human hyaluronidases such as Hyall, or truncated forms of non-human or human membrane-associated hyaluronidases, in particular truncated forms of human PH20, allelic variants thereof and other variants thereof. Exemplary of hyaluronan-degrading enzymes in the co-formulations herein are truncated forms of a hyaluronan-degrading enzyme that lack one or more amino acid residues of a glycosylphosphatidylinositol (GPI) anchor and that retain hyaluornidase activity. In one example, the human hyaluronidase PH20, which is normally membrane anchored via a GPI anchor, can be made soluble by truncation of and removal of all or a portion of the GPI anchor at the C-terminus.

Thus, in some instances, a hyaluronan degrading enzyme that is normally GPI-anchored (such as, for example, human PH20) is rendered soluble by truncation at the C-terminus. Such truncation can remove all of the GPI anchor attachment signal sequence, or can remove only some of the GPI anchor attachment signal sequence. The resulting polypeptide, however, is soluble. In instances where the soluble hyaluronan degrading enzyme retains a portion of the GPI anchor attachment signal sequence, 1, 2, 3, 4, 5, 6, 7, 8, 9, 10 or more amino acid residues in the GPI-anchor attachment signal sequence can be retained, provided the polypeptide is soluble (i.e. secreted when expressed from cells) and active. One of skill in the art can determine whether a polypeptide is GPI-anchored using methods well known in the art. Such methods include, but are not limited to, using known algorithms to predict the presence and location of the GPI-anchor attachment signal sequence and co-site, and performing solubility analyses before and after digestion with phosphatidylinositol-specific phospholipase C(PI-PLC) or D (PI-PLD).

Exemplary of a soluble hyaluronidase is PH20 from any species, such as any set forth in any of SEQ ID NOS: 1, 2, 11, 25, 27, 30-32, 63-65 and 185-186, or truncated forms thereof lacking all or a portion of the C-terminal GPI anchor, so long as the hyaluronidase is soluble and retains hyaluronidase activity. Exemplary soluble hyaluronidases that are C-terminally truncated and lack all or a portion of the GPI anchor attachment signal sequence include, but are not limited to, PH20 polypeptides of primate origin, such as, for example, human and chimpanzee PH20 polypeptides. For example, soluble PH20 polypeptides can be made by C-terminal truncation of any of the mature or precursor polypeptides set forth in SEQ ID NOS:1, 2 or 185, or allelic or other variation thereof, including active fragment thereof, wherein the resulting polypeptide is soluble and lacks all or a portion of amino acid residues from the GPI-anchor attachment signal sequence. Also included among soluble hyaluronidases are allelic variants or other variants of any of SEQ ID NOS: 1, 2, 11, 25, 27, 30-32, 63-65 and 185-186, or truncated forms thereof. Allelic variants and other variants are known to one of skill in the art, and include polypeptides having 60%, 70%, 80%, 90%, 91%, 92%, 93%, 94%, 95%. 96%. 97%. 98% or more sequence identity to any of SEQ ID NOS: 1, 2, 11, 25, 27, 30-32, 63-65 and 185-186, or truncated forms thereof. Amino acid variants include conservative and non-conservative mutations. It is understood that residues that are important or otherwise required for the activity of a hyaluronidase, such as any described above or known to skill in the art, are generally invariant and cannot be changed. These include, for example, active site residues. Thus, for example, amino acid residues 111, 113 and 176 (corresponding to residues in the mature PH20 polypeptide set forth in SEQ ID NO:2) of a human PH20 polypeptide, or soluble form thereof, are generally invariant and are not altered. Other residues that confer glycosylation and formation of disulfide bonds required for proper folding also can be invariant.

a. C-Terminal Truncated Human PH20

Exemplary of a soluble hyaluronidase is a C-terminal truncated human PH20. C-terminal truncated forms of recombinant human PH20 have been produced and can be used in the co-formulations described herein. The production of such soluble forms of PH20 is described in U.S. Pat. No. 7,767,429 and U.S. Pat. Application Nos. US20040268425; US 20050260186, US20060104968 and US20100143457.

For example, C-terminal truncated PH20 polypeptides include polypeptides that at least contain amino acids 36-464 (the minimal portion required for hyaluronidase activity), or include a sequence of amino acids that has at least 85%, for example at least 86%, 87%, 88%, 89%, 90%, 91%, 92%, 93%, 94%, 95%, 95%, 97%, 98% sequence identity to a sequence of amino acids that includes at least amino acids 36-464 of SEQ ID NO:1 and retain hyaluronidase activity. Included among these polypeptides are human PH20 polypeptides that completely lack all the GPI-anchor attachment signal sequence. Also include among these polypeptides are human PH20 polypeptides that lack a portion of contiguous amino acid residues of the GPI-anchor attachment signal sequence (termed extended soluble PH20 (esPH20); see e.g. US20100143457). C-terminally truncated PH20 polypeptides can be C-terminally truncated by 1, 2, 3, 4, 5, 6, 7, 8, 9, 10, 11, 12, 13, 14, 15, 16, 17, 18, 19, 20, 25, 30, 35, 40, 45, 50, 55, 60 or more amino acids compared to the full length wild type polypeptide, such as a full length wild type polypeptide with a sequence set forth in SEQ ID NOS:1 or 2, or allelic or species variants or other variants thereof. Thus, instead of having a GPI-anchor covalently attached to the C-terminus of the protein in the ER and being anchored to the extracellular leaflet of the plasma membrane, these polypeptides are secreted when expressed from cells and are soluble.

Exemplary C-terminally truncated human PH20 polypeptides provided herein include any that include at least amino acids 36-464 of SEQ ID NO:1 and are C-terminally truncated after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1, or a variant thereof that exhibits at least 85% sequence identity, such as at least 86%, 87%, 88%, 89%, 90%, 91%, 92%, 93%, 94%, 95%, 95%, 97%, 98% sequence identity thereto and retains hyaluronidase activity. Table 4 provides non-limiting examples of exemplary C-terminally truncated PH20 polypeptides. In Table 4 below, the length (in amino acids) of the precursor and mature polypeptides, and the sequence identifier (SEQ ID NO) in which exemplary amino acid sequences of the precursor and mature polypeptides of the C-terminally truncated PH20 proteins are set forth, are provided. The wild-type PH20 polypeptide also is included in Table 4 for comparison.

TABLE 4
Exemplary C-terminally truncated PH20 polypeptides

| | Precursor | Precursor | Mature | Mature |
|---|---|---|---|---|
| | (amino | SEQ | (amino | SEQ |
| Polypeptide | acids) | ID NO | acids) | ID NO |
| wildtype | 509 | 1 | 474 | 2 |
| SPAM1-SILF | 500 | 223 | 465 | 267 |
| SPAM-VSIL | 499 | 190 | 464 | 234 |
| SPAM1-IVSI | 498 | 224 | 463 | 268 |
| SPAM1-FIVS | 497 | 191 | 462 | 235 |
| SPAM1-MFIV | 496 | 225 | 461 | 269 |
| SPAM1-TMFI | 495 | 192 | 460 | 236 |
| SPAM1-ATMF | 494 | 226 | 459 | 270 |
| SPAM1-SATM | 493 | 193 | 458 | 237 |
| SPAM1-LSAT | 492 | 227 | 457 | 271 |
| SPAM1-TLSA | 491 | 194 | 456 | 238 |
| SPAM1-STLS | 490 | 196 | 455 | 240 |
| SPAM1-PSTL | 489 | 195 | 454 | 239 |
| SPAM1-SPST | 488 | 228 | 453 | 272 |
| SPAM1-ASPS | 487 | 197 | 452 | 241 |
| SPAM1-NASP | 486 | 229 | 451 | 273 |
| SPAM1-YNAS | 485 | 198 | 450 | 242 |
| SPAM1-FYNA | 484 | 199 | 449 | 243 |
| SPAM1-IFYN | 483 | 46 | 448 | 48 |
| SPAM1-QIFY | 482 | 3 | 447 | 4 |
| SPAM1-PQIF | 481 | 45 | 446 | 5 |
| SPAM1-EPQI | 480 | 44 | 445 | 6 |
| SPAM1-EEPQ | 479 | 43 | 444 | 7 |
| SPAM1-TEEP | 478 | 42 | 443 | 8 |
| SPAM1-ETEE | 477 | 41 | 442 | 9 |
| SPAM1-METE | 476 | 200 | 441 | 244 |
| SPAM1-PMET | 475 | 201 | 440 | 245 |
| SPAM1-PPME | 474 | 202 | 439 | 246 |
| SPAM1-KPPM | 473 | 203 | 438 | 247 |
| SPAM1-LKPP | 472 | 204 | 437 | 248 |
| SPAM1-FLKP | 471 | 205 | 436 | 249 |
| SPAM1-AFLK | 470 | 206 | 435 | 250 |
| SPAM1-DAFL | 469 | 207 | 434 | 251 |
| SPAM1-IDAF | 468 | 208 | 433 | 252 |
| SPAM1-CIDA | 467 | 40 | 432 | 47 |
| SPAM1-VCID | 466 | 209 | 431 | 253 |
| SPAM1-GVCI | 465 | 200 | 430 | 254 |

b. rHuPH20

Exemplary of a C-terminal truncated form of SEQ ID NO:1 is a polypeptide thereof that is truncated after amino acid 482 of the sequence set forth in SEQ ID NO:1. Such a polypeptide can be generated from a nucleic acid molecule encoding amino acids 1-482 (set forth in SEQ ID NO:3). Such an exemplary nucleic acid molecule is set forth in SEQ ID NO:49. Post translational processing removes the 35 amino acid signal sequence, leaving a 447 amino acid soluble recombinant human PH20 (SEQ ID NO:4). As produced in the culture medium there is heterogeneity at the C-terminus such that the product, designated rHuPH20, includes a mixture of species that can include any one or more of SEQ ID NOS:4-9 in various abundance. Typically, rHuPH20 is produced in cells that facilitate correct N-glycosylation to retain activity, such as CHO cells (e.g. DG44 CHO cells).

4. Glycosylation of Hyaluronan Degrading Enzymes

Glycosylation, including N- and O-linked glycosylation, of some hyaluronan degrading enzymes, including hyaluronidases, can be important for their catalytic activity and stability. While altering the type of glycan modifying a glycoprotein can have dramatic affects on a protein's antigenicity, structural folding, solubility, and stability, most enzymes are not thought to require glycosylation for optimal enzyme activity. For some hyaluronidases, removal of N-linked glycosylation can result in near complete inactivation of the hyaluronidase activity. Thus, for such hyaluronidases, the presence of N-linked glycans is critical for generating an active enzyme.

N-linked oligosaccharides fall into several major types (oligomannose, complex, hybrid, sulfated), all of which have (Man)-3-GlcNAc-GlcNAc-cores attached via the amide nitrogen of Asn residues that fall within -Asn-Xaa-Thr/Ser-sequences (where Xaa is not Pro). Glycosylation at an -Asn-Xaa-Cys- site has been reported for coagulation protein C. In some instances, a hyaluronan degrading enzyme, such as a hyaluronidase, can contain both N-glycosidic and O-glycosidic linkages. For example, PH20 has O-linked oligosaccharides as well as N-linked oligosaccharides. There are seven potential N-linked glycosylation sites at N82, N166, N235, N254, N368, N393, N490 of human PH20 exemplified in SEQ ID NO: 1. Amino acid residues N82, N166 and N254 are occupied by complex type glycans whereas amino acid residues N368 and N393 are occupied by high mannose type glycans. Amino acid residue N235 is occupied by approximately 80% high mannose type glycans and 20% complex type glycans. As noted above, N-linked glycosylation at N490 is not required for hyaluronidase activity.

In some examples, the hyaluronan degrading enzymes for use herein are glycosylated at one or all of the glycosylation sites. For example, for human PH20, or a soluble form thereof, 2, 3, 4, 5, or 6 of the N-glycosylation sites corresponding to amino acids N82, N166, N235, N254, N368, and N393 of SEQ ID NO:1 are glycosylated. In some examples the hyaluronan degrading enzymes are glycosylated at one or more native glycosylation sites. Generally soluble forms of PH20 are produced using protein expression systems that facilitate correct N-glycosylation to ensure the polypeptide retains activity, since glycosylation is important for the catalytic activity and stability of hyaluronidases. Such cells include, for example Chinese Hamster Ovary (CHO) cells (e.g. DG44 CHO cells).

In other examples, the hyaluronan degrading enzymes are modified at one or more non-native glycosylation sites to confer glycosylation of the polypeptide at one or more additional site. In such examples, attachment of additional sugar moieties can enhance the pharmacokinetic properties of the molecule, such as improved half-life and/or improved activity.

In other examples, the hyaluronan degrading enzymes, such as a PH20 or human PH20, used in the methods provided herein are partially deglycosylated (or N-partially glycosylated polypeptides) (see e.g. US20100143457). Glycosidases, or glycoside hydrolases, are enzymes that catalyze the hydrolysis of the glycosidic linkage to generate two smaller sugars. The major types of N-glycans in vertebrates include high mannose glycans, hybrid glycans and complex glycans. There are several glycosidases that result in only partial protein deglycosylation, including: EndoF1, which cleaves high mannose and hybrid type glycans; EndoF2, which cleaves biantennary complex type glycans; EndoF3, which cleaves biantennary and more branched complex glycans; and EndoH, which cleaves high mannose and hybrid type glycans. For example, treatment of PH20 (e.g. a recombinant PH20 designated rHuPH20) with one or all of the above glycosidases (e.g. EndoF1, EndoF2 EndoF3 and/or EndoH) results in partial deglycosylation. These partially deglycosylated PH20 polypeptides can exhibit hyaluronidase enzymatic activity that is comparable to the fully glycosylated polypeptides. In contrast, treatment of PH20 with PNGaseF, a glycosidase that cleaves all N-glycans, or with the GlcNAc phosphotransferase (GPT) inhibitor tunicamycin, results in complete deglycosylation of all N-glycans and thereby renders PH20 enzymatically inactive. Thus, although all N-linked glycosylation sites (such as, for example, those at amino acids N82, N166, N235, N254, N368, and N393 of human PH20, exemplified in SEQ ID NO:1) can be glycosylated, treatment with one or more glycosidases can render the extent of glycosylation reduced compared to a hyaluronidase that is not digested with one or more glycosidases.

Hence, partially deglycosylated hyaluronan degrading enzymes, such as partially deglycosylated soluble hyaluronidases, can be produced by digestion with one or more glycosidases, generally a glycosidase that does not remove all N-glycans but only partially deglycosylates the protein. The partially deglycosylated hyaluronan degrading enzymes, including partially deglycosylated soluble PH20 polypeptides, can have 10%, 20%, 30%, 40%, 50%, 60%, 70% or 80% of the level of glycosylation of a fully glycosylated polypeptide. In one example, 1, 2, 3, 4, 5 or 6 of the N-glycosylation sites corresponding to amino acids N82, N166, N235, N254, N368, and N393 of SEQ ID NO:1 are partially deglycosylated, such that they no longer contain high mannose or complex type glycans, but rather contain at least an N-acetylglucosamine moiety. In some examples, 1, 2 or 3 of the N-glycosylation sites corresponding to amino acids N82, N166 and N254 of SEQ ID NO:1 are deglycosylated, that is, they do not contain a sugar moiety. In other examples, 3, 4, 5, or 6 of the N-glycosylation sites corresponding to amino acids N82, N166, N235, N254, N368, and N393 of SEQ ID NO:1 are glycosylated. Glycosylated amino acid residues minimally contain an N-acetylglucosamine moiety. Typically, the partially deglycosylated hyaluronan degrading enzymes, including partially deglycosylated soluble PH20 polypeptides, exhibit hyaluronidase activity that is 10%, 20%, 30%, 40%, 50%, 60%, 70%, 80%, 90%, 100%, 110%, 120%, 130%, 140%, 150%, 200%, 300%, 400%, 500%, 1000% or more of the hyaluronidase activity exhibited by the fully glycosylated polypeptide.

5. Modifications of Hyaluronan Degrading Enzymes to Improve their Pharmacokinetic Properties

Hyaluronan degrading enzymes can be modified to improve their pharmacokinetic properties, such as increasing their half-life in vivo and/or activities. The modification of hyaluronan degrading enzymes for use in the methods provided herein can include attaching, directly or indirectly via a linker, such as covalently or by other stable linkage, a polymer, such as dextran, a polyethylene glycol (pegylation(PEG)) or sialyl moiety, or other such polymers, such as natural or sugar polymers.

Pegylation of therapeutics is known to increase resistance to proteolysis, increase plasma half-life, and decrease antigenicity and immunogenicity. Covalent or other stable attachment (conjugation) of polymeric molecules, such as polyethylene glycol moiety (PEG), to the hyaluronan degrading enzyme thus can impart beneficial properties to the resulting enzyme-polymer composition. Such properties include improved biocompatibility, extension of protein (and enzymatic activity) half-life in the blood, cells and/or in other tissues within a subject, effective shielding of the protein from proteases and hydrolysis, improved biodistribution, enhanced pharmacokinetics and/or pharmacodynamics, and increased water solubility.

Exemplary polymers that can be conjugated to the hyaluronan degrading enzyme, include natural and synthetic homopolymers, such as polyols (i.e. poly-OH), polyamines (i.e. poly-NH₂) and polycarboxyl acids (i.e. poly-COOH), and further heteropolymers i.e. polymers comprising one or more different coupling groups e.g. a hydroxyl group and amine groups. Examples of suitable polymeric molecules include polymeric molecules selected from among polyalkylene oxides (PAO), such as polyalkylene glycols (PAG), including polypropylene glycols (PEG), methoxypolyethylene glycols (mPEG) and polypropylene glycols, PEG-glycidyl ethers (Epox-PEG), PEG-oxycarbonylimidazole (CDI-PEG) branched polyethylene glycols (PEGs), polyvinyl alcohol (PVA), polycarboxylates, polyvinylpyrrolidone, poly-D, L-amino acids, polyethylene-co-maleic acid anhydride, polystyrene-co-maleic acid anhydride, dextrans including carboxymethyl-dextrans, heparin, homologous albumin, celluloses, including methylcellulose, carboxymethylcellulose, ethylcellulose, hydroxyethylcellulose, carboxyethylcellulose and hydroxypropylcellulose, hydrolysates of chitosan, starches such as hydroxyethyl-starches and hydroxypropyl-starches, glycogen, agaroses and derivatives thereof, guar gum, pullulan, inulin, xanthan gum, carrageenan, pectin, alginic acid hydrolysates and bio-polymers.

Typically, the polymers are polyalkylene oxides (PAO), such as polyethylene oxides, such as PEG, typically mPEG, which, in comparison to polysaccharides such as dextran, pullulan and the like, have few reactive groups capable of cross-linking. Typically, the polymers are non-toxic polymeric molecules such as (m)polyethylene glycol (mPEG) which can be covalently conjugated to the hyaluronan degrading enzyme (e.g., to attachment groups on the protein surface) using relatively simple chemistry.

Suitable polymeric molecules for attachment to the hyaluronan degrading enzyme include, but are not limited to, polyethylene glycol (PEG) and PEG derivatives such as methoxy-polyethylene glycols (mPEG), PEG-glycidyl ethers (Epox-PEG), PEG-oxycarbonylimidazole (CDI-PEG), branched PEGs, and polyethylene oxide (PEO) (see e.g. Roberts et al., Advanced Drug Delivery Review 2002, 54: 459-476; Harris and Zalipsky, S (eds.) “Poly(ethylene glycol), Chemistry and Biological Applications” ACS Symposium Series 680, 1997; Mehvar et al., J. Pharm. Pharmaceut. Sci., 3(1):125-136, 2000; Harris, Nature Reviews 2:215 (2003); and Tsubery, J Biol. Chem. 279(37):38118-24, 2004). The polymeric molecule can be of a molecular weight typically ranging from about 3 kDa to about 60 kDa. In some embodiments the polymeric molecule that is conjugated to a protein, such as rHuPH20, has a molecular weight of 5, 10, 15, 20, 25, 30, 35, 40, 45, 50, 55, 60 or more than 60 kDa.

Various methods of modifying polypeptides by covalently attaching (conjugating) a PEG or PEG derivative (i.e. “PEGylation”) are known in the art (see e.g., U.S. Pat. Pub. Nos. 20060104968 and U.S. 20040235734; U.S. Pat. No. 5,672,662 and U.S. Pat. No. 6,737,505). Techniques for PEGylation include, but are not limited to, specialized linkers and coupling chemistries (see e.g., Harris, Adv. Drug Deliv. Rev. 54:459-476, 2002), attachment of multiple PEG moieties to a single conjugation site (such as via use of branched PEGs; see e.g., Veronese et al., Bioorg. Med. Chem. Lett. 12:177-180, 2002), site-specific PEGylation and/or mono-PEGylation (see e.g., Chapman et al., Nature Biotech. 17:780-783, 1999), and site-directed enzymatic PEGylation (see e.g., Sato, Adv. Drug Deliv. Rev., 54:487-504, 2002) (see, also, for example, Lu and Felix (1994) Int. J. Peptide Protein Res. 43:127-138; Lu and Felix (1993) Peptide Res. 6:142-6, 1993; Felix et al. (1995) Int. J.

Peptide Res. 46:253-64; Benhar et al. (1994) J. Biol. Chem. 269:13398-404; Brumeanu et al. (1995) J Immunol. 154:3088-95; see also, Caliceti et al. (2003) Adv. Drug Deliv. Rev. 55(10):1261-77 and Molineux (2003) Pharmacotherapy 23 (8 Pt 2):3S-8S). Methods and techniques described in the art can produce proteins having 1, 2, 3, 4, 5, 6, 7, 8, 9, 10 or more than 10 PEG or PEG derivatives attached to a single protein molecule (see e.g., U.S. Pat. Pub. No. 20060104968).

Numerous reagents for PEGylation have been described in the art. Such reagents include, but are not limited to, N-hydroxysuccinimidyl (NHS) activated PEG, succinimidyl mPEG, mPEG2-N-hydroxysuccinimide, mPEG succinimidyl alpha-methylbutanoate, mPEG succinimidyl propionate, mPEG succinimidyl butanoate, mPEG carboxymethyl 3-hydroxybutanoic acid succinimidyl ester, homobifunctional PEG-succinimidyl propionate, homobifunctional PEG propionaldehyde, homobifunctional PEG butyraldehyde, PEG maleimide, PEG hydrazide, p-nitrophenyl-carbonate PEG, mPEG-benzotriazole carbonate, propionaldehyde PEG, mPEG butryaldehyde, branched mPEG2 butyraldehyde, mPEG acetyl, mPEG piperidone, mPEG methylketone, mPEG “linkerless” maleimide, mPEG vinyl sulfone, mPEG thiol, mPEG orthopyridylthioester, mPEG orthopyridyl disulfide, Fmoc-PEG-NHS, Boc-PEG-NHS, vinylsulfone PEG-NHS, acrylate PEG-NHS, fluorescein PEG-NHS, and biotin PEG-NHS (see e.g., Monfardini et al., Bioconjugate Chem. 6:62-69, 1995; Veronese et al., J. Bioactive Compatible Polymers 12:197-207, 1997; U.S. Pat. No. 5,672,662; U.S. Pat. No. 5,932,462; U.S. Pat. No. 6,495,659; U.S. Pat. No. 6,737,505; U.S. Pat. No. 4,002,531; U.S. Pat. No. 4,179,337; U.S. Pat. No. 5,122,614; U.S. Pat. No. 5,324,844; U.S. Pat. No. 5,446,090; U.S. Pat. No. 5,612,460; U.S. Pat. No. 5,643,575; U.S. Pat. No. 5,766,581; U.S. Pat. No. 5,795,569; U.S. Pat. No. 5,808,096; U.S. Pat. No. 5,900,461; U.S. Pat. No. 5,919,455; U.S. Pat. No. 5,985,263; U.S. Pat. No. 5,990,237; U.S. Pat. No. 6,113,906; U.S. Pat. No. 6,214,966; U.S. Pat. No. 6,258,351; U.S. Pat. No. 6,340,742; U.S. Pat. No. 6,413,507; U.S. Pat. No. 6,420,339; U.S. Pat. No. 6,437,025; U.S. Pat. No. 6,448,369; U.S. Pat. No. 6,461,802; U.S. Pat. No. 6,828,401; U.S. Pat. No. 6,858,736; U.S. 2001/0021763; U.S. 2001/0044526; U.S. 2001/0046481; U.S. 2002/0052430; U.S. 2002/0072573; U.S. 2002/0156047; U.S. 2003/0114647; U.S. 2003/0143596; U.S. 2003/0158333; U.S. 2003/0220447; U.S. 2004/0013637; US 2004/0235734; U.S. 2005/0114037; U.S. 2005/0171328; U.S. 2005/0209416; EP 01064951; EP 0822199; WO 00176640; WO 0002017; WO 0249673; WO 0500360; WO 9428024; and WO 0187925).

### F. SUPER FAST-ACTING INSULIN FORMULATIONS, AND STABLE FORMULATIONS THEREOF

Super-fast acting insulin compositions are co-formulations containing a fast-acting insulin, such as a fast-acting insulin analog (or rapid acting analog), and a hyaluronan-degrading enzyme. Such compositions can be used in the CSII methods herein. A super-fast acting insulin composition provides an ultra-fast insulin response that more closely mimics the endogenous (i.e. natural) post-prandial insulin release of a nondiabetic subject compared to conventional fast-acting insulins, such as insulin analogs. Such super-fast acting insulin compositions are known in the art (see e.g. U.S. publication No. US20090304665).

A super-fast acting insulin compositions contains a therapeutically effective amount of a fast-acting insulin for controlling blood glucose levels and an amount of a hyaluronan-degrading enzyme sufficient to render the composition a super fast-acting insulin composition. Any fast-acting insulin described in Section D and any hyaluronan-degrading enzyme described in Section E can be combined in a co-formulation to generate a super fast-acting insulin composition so long as the resulting composition effects an ultra-fast insulin response when administered.

Generally, the amount of a fast-acting insulin in a super-fast acting insulin composition is from or from about 10 U/mL to 1000 U/mL, and the amount of a hyaluronan-degrading enzyme is functionally equivalent to 1 U/mL to 10,000 U/mL. For example, the amount of a fast-acting insulin is or is about or at least 100 U/mL and the amount of a hyaluronan-degrading enzyme is functionally equivalent to or about to or at least 600 U/mL. In some examples where the fast-acting insulin is a regular insulin, insulin lispro, insulin aspart or insulin glulisine or other similarly sized fast-acting insulin, the amount of insulin in the super-fast acting composition is from or from about 0.35 mg/mL to 35 mg/mL.

In particular examples, the hyaluronan-degrading enzyme is a stable co-formulation as described in U.S. provisional application No. 61/520,962 and entitled “Stable co-formulations of a hyaluronan-degrading enzyme and insulin.” In particular examples, for purposes of continuous subcutaneous infusion, a super-fast acting insulin composition is stable for at least 3 days at a temperature from or from about 32° C. to 40° C.

1. Stable Co-Formulations

The co-formulations provided herein contain a therapeutically effective amount of a fast-acting insulin, such as a rapid acting insulin analog (e.g. insulin lispro, insulin aspart or insulin glulisine). For example, the co-formulations contain a fast-acting insulin in an amount between or about between 10 U/mL to 1000 U/mL, 100 U/mL to 1000 U/mL, or 500 U/mL to 1000 U/mL, such as at least or about at least 10 U/mL, 20 U/mL, 30 U/mL, 40 U/mL, 50 U/mL, 60 U/mL, 70 U/mL, 80 U/mL, 90 U/mL, 100 U/mL, 150 U/mL, 200 U/mL, 250 U/mL, 300 U/mL, 350 U/mL, 400 U/mL, 450 U/ml, 500 U/mL or 1000 U/mL. For example, the co-formulations provided herein contain a fast-acting insulin, such as a rapid acting insulin analog (e.g. insulin lispro, insulin aspart or insulin glulisine) in an amount that is at least or at least about 100 U/mL.

The amount of hyaluronan degrading enzyme, such as a hyaluronidase for example a PH20 (e.g. rHuPH20), in the stable co-formulations is an amount that renders the composition super-fast acting. For example, the hyaluronan-degrading enzyme is in an amount that is functionally equivalent to at least or about at least 30 Units/mL. For example, the stable co-formulations contain a hyaluronan-degrading enzyme, such as a hyaluronidase for example a PH20 (e.g. rHuPH20) in an amount between or about between 30 Units/mL to 3000 U/mL, 300 U/mL to 2000 U/mL or 600 U/mL to 2000 U/mL or 600 U/mL to 1000 U/mL, such as at least or about at least 30 U/mL, 35 U/mL, 40 U/mL, 50 U/mL, 100 U/mL, 200 U/mL, 300 U/mL, 400 U/mL, 500 U/mL, 600 U/mL, 700 U/mL, 800 U/mL, 900 U/mL, 1000 U/ml, 2000 U/mL or 3000 U/mL. For example, the co-formulations provided herein contain a PH20 (e.g. rHuPH20) that is in an amount that is at least 100 U/mL to 1000 U/mL, for example at least or about at least or about or 600 U/mL.

The volume of the stable co-formulations can be any volume suitable for the container in which it is provided. In some examples, the co-formulations are provided in a vial, syringe, pen, reservoir for a pump or a closed loop system, or any other suitable container. For example, the co-formulations provided herein are between or about between 0.1 mL to 500 mL, such as 0.1 mL to 100 mL, 1 mL to 100 mL, 0.1 mL to 50 mL, such as at least or about at least or about or 0.1 mL, 1 mL, 2 mL, 3 mL, 4 mL, 5 mL, 10 mL, 15 mL, 20 mL, 30 mL, 40 mL, 50 mL or more.

In the stable co-formulations, the stability of the insulin, including insulin analogs, in the formulations is a function of the recovery, purity and/or activity of the insulin under storage at temperatures of at least or about 32° C. to 40° C. The formulations provided herein retain insulin recovery, purity and/or activity such that the formulations are suitable for therapeutic use as described herein. For example, in the formulations provided herein, the insulin purity (e.g. as assessed by RP-HPLC or other similar method) over time and under storage or use conditions as described herein is at least 90% of the purity, potency or recovery of insulin in the formulation prior to storage or use, for example, at least 90%, 91%, 92%, 93%, 94%, 95%, 96%, 97%, 98%, 99% or more. Generally, for insulin purity (e.g. by RP-HPLC) the target acceptable specification is at least or about 90% purity or about or greater than 90% purity. In other examples, insulin purity can be assessed as a function of aggregation of the insulin, for example, using non-denaturing or denaturing size exclusion chromatography (SEC). In such examples, in the co-formulations provided herein contain less than 2% high molecular weight (HMWt) insulin species by peak area, for example, less than 1.9%, 1.8%, 1.7%, 1.6%, 1.5%, 1.4%, 1.3%, 1.2%, 1.1%, 1.0% or less.

In the stable co-formulations, the stability of a hyaluronan-degrading enzyme, including a hyaluronidase such as a PH20 (e.g. rHuPH20), in the formulations is a function of the recovery and/or activity of the enzyme under storage at temperatures of at least or about 32° C. to 40° C. The formulations provided herein retain hyaluronidase recovery and/or activity such that the formulations are suitable for therapeutic use as described herein. In the stable co-formulations provided herein, the activity of the hyaluronan degrading enzyme, such as a hyaluronidase, for example a PH20, typically is greater than 50% of the initial hyaluronidase activity for at least 3 days at a temperature from or from about 32° C. to 40° C., such as at least or greater than 55%, 60%, 65%, 70%, 80%, 90%, 91%, 92%, 93%, 94%, 95%, 96%, 97%, 98%, 99% or more. Generally, for hyaluronidase activity the target acceptable specification for stability is at least 62% of the activity of the enzyme. Thus, for example, in a solution formulated with 600 U/mL of a hyaluronan-degrading enzyme, for example rHuPH20, at least or about at least 360 Units/mL, 365 U/mL, 370 U/mL, 375 U/mL, 380 U/mL, 390 U/mL, 420 U/mL, 480 U/mL, 540 U/mL, 546 U/mL, 552 U/mL, 558 U/mL, 564 U/mL, 570 U/mL, 576 U/mL, 582 U/mL, 588 U/mL, 594 U/mL or more activity is retained over time and under storage or use conditions. In other examples, stability can be assessed as a function of recovery of the enzyme, for example, using RP-HPLC. In such examples, the hyaluronidase enzyme recovery in the stable co-formulations provided herein is from between or about between 60% to 140%. For example, in the formulations provided herein the hyaluronidase enzyme recovery is from between or about between 3-7 μg/mL.

Typically, the compounds are formulated into pharmaceutical compositions using techniques and procedures well known in the art (see e.g., Ansel Introduction to Pharmaceutical Dosage Forms, Fourth Edition, 1985, 126). Pharmaceutically acceptable compositions are prepared in view of approvals for a regulatory agency or other agency prepared in accordance with generally recognized pharmacopeia for use in animals and in humans. The formulation should suit the mode of administration.

The stable co-formulations can be provided as a pharmaceutical preparation in liquid form as solutions, syrups or suspensions. In liquid form, the pharmaceutical preparations can be provided as a concentrated preparation to be diluted to a therapeutically effective concentration before use. Generally, the preparations are provided in a dosage form that does not require dilution for use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). In another example, pharmaceutical preparations can be presented in lyophilized form for reconstitution with water or other suitable vehicle before use.

Provided below is a description of the further components, besides insulin and hyaluronan-degrading enzyme, that are provided in the stable co-formulations herein. The particular balance of requirements to maximize stability of both proteins as contained in the co-formulations provided herein continuous subcutaneous infusion of the co-formulation for at least 3 days achievable, while maintaining stability of the proteins. A description of each of the components or conditions, such as excipients, stabilizers or pH, is provided below.

Typically, the stable co-formulation composition has a pH of between or about between 6.5 to 7.5 and also contains NaCl at a concentration between or about between 120 mM to 200 mM, an anti-microbial effective amount of a preservative or mixture of preservatives, a stabilizing agent or agents.

a. NaCl and pH

In particular, it is found herein that although insulin crystallizes at 2° C. to 8° C. at high salt concentrations and low pH, it does not crystallize at high salt concentrations and low pH at higher temperatures of 32° C. to 40° C. Accordingly, the opposing requirement of high salt concentration and low pH required by a hyaluronan-degrading enzymes (e.g. PH20) to maintain its stability at high temperatures of 32° C. to 40° C. is more compatible at higher temperatures for at least a short period of time of at least 3 days. Also, the same high salt and low pH formulations confer similar stability between and among the insulin analogs, despite differences in apparent solubility that affect stability of insulin at the lower temperatures.

For example, co-formulations provided herein that are stable at elevated temperature of 32° C. to 40° C. for at least 3 days contain 120 mM to 200 mM NaCl, such as 150 mM NaCl to 200 mM NaCl or 160 mM NaCl to 180 mM NaCl, for example at or about 120 mM, 130 mM, 140 mM, 150 mM, 155 mM, 160 mM, 165 mM, 170 mM, 175 mM, 180 mM, 185 mM, 190 mM, 195 mM or 200 mM NaCl.

Also, the co-formulations provided herein that are stable under elevated temperature of 32° C. to 40° C. for at least 3 days contain a pH of 6.5 to 7.5 or 6.5 to 7.2, such as or about a pH of 6.5±0.2, 6.6±0.2, 6.7±0.2, 6.8±0.2, 6.9±0.2 7.0 0.2, 7.1 0±0.2, 7.2 0±0.2, 7.3±0.2, 7.4±0.2 or 7.5±0.2. Insulin solubility, particularly at refrigerated temperatures, decreases in these reduced pH and increased salt conditions. Thus such formulations typically are not stored at refrigerated or ambient temperatures prior to use.

b. Hyaluronidase Inhibitor

In another example, the stable co-formulations contain as a stabilizing agent a hyaluronidase inhibitor to stabilize the hyaluronan-degrading enzyme in the co-formulation. In particular examples, the hyaluronidase inhibitor is one that reacts with insulin or hyaluronan-degrading enzyme in an associative and non-covalent manner, and does not form covalent complexes with insulin or a hyaluronan-degrading enzyme. The hyaluronidase inhibitor is provided at least at its equilibrium concentration. One of skill in the art is familiar with various classes of hyaluronidase inhibitors (see e.g. Girish et al. (2009) Current Medicinal Chemistry, 16:2261-2288, and references cited therein). One of skill in the art knows or can determine by standard methods in the art the equilibrium concentration of a hyaluronidase inhibitor in a reaction or stable composition herein. The choice of hyaluronidase inhibitor will depend on the particular hyaluronan-degrading enzyme used in the composition. For example, hyaluronan is an exemplary hyaluronidase inhibitor for use in the stable compositions herein when the hyaluronan-degrading enzyme is a PH20.

Exemplary hyaluronidase inhibitors for use as stabilizing agents herein include, but are not limited to, a protein, glycosaminoglycan (GAG), polysaccharides, fatty acid, lanostanoids, antibiotics, anti-nematodes, synthetic organic compounds or a plant-derived bioactive component. For example, a hyaluronidase plant-derived bioactive component can be an alkaloid, antioxidant, polyphenol, flavonoids, terpenoids and anti-inflammatory drugs. Exemplary hyaluronidase inhibitors include, for example, serum hyaluronidase inhibitor, Withania somnifera glycoprotein (WSG), heparin, heparin sulfate, dermatan sulfate, chitosans, β-(1,4)-galacto-oligosaccharides, sulphated verbascose, sulphated planteose, pectin, poly(styrene-4-sulfonate), dextran sulfate, sodium alginate, polysaccharide from Undaria pinnatifida, mandelic acid condensation polymer, eicosatrienoic acid, nervonic acid, oleanolic acid, aristolochic acid, ajmaline, reserpine, flavone, desmethoxycentauredine, quercetin, apigenin, kaempferol, silybin, luteolin, luteolin-7-glucoside, phloretin, apiin, hesperidin, sulphonated hesperidin, calycosin-7-O-β-D-glucopyranoside, sodium flavone-7-sulphate, flavone 7-fluoro-4′-hydroxyflavone, 4′-chloro-4,6-dimethoxychalcone, sodium 5-hydroxyflavone 7-sulphate, myricetin, rutin, morin, glycyrrhizin, vitamin C, D-isoascorbic acid, D-saccharic 1,4-lactone, L-ascorbic acid-6-hexadecanoate (Vcpal), 6-O-acylated vitamin C, catechin, nordihydroguaiaretic acid, curcumin, N-propyl gallate, tannic acid, ellagic acid, gallic acid, phlorofucofuroeckol A, dieckol, 8,8′-bieckol, procyanidine, gossypol, celecoxib, nimesulide, dexamethasone, indomethcin, fenoprofen, phenylbutazone, oxyphenbutazone, salicylates, disodium cromoglycate, sodium aurothiomalate, transilist, traxanox, ivermectin, linocomycin and spectinomycin, sulfamethoxazole and trimerthoprim, neomycin sulphate, 3α-acetylpolyporenic acid A, (25S)-(+)-12α-hydroxy-3α-methylcarboxyacetate-24-methyllanosta-8,24(31)-diene-26-oic acid, lanostanoid, polyporenic acid c, PS53 (hydroquinone-sulfonic acid-formaldehyde polymer), polymer of poly (styrene-4-sulfonate), VERSA-TL 502, 1-tetradecane sulfonic acid, mandelic acid condensation polymer (SAMMA), 1,3-diacetylbenzimidazole-2-thione, N-monoacylated benzimidazol-2thione, N,N′-diacylated benzimidazol-2-thione, alkyl-2-phenylindole derivate, 3-propanoylbenzoxazole-2-thione, N-alkylated indole derivative, N-acylated indole derivate, benzothiazole derivative, N-substituted indole-2- and 3-carboxamide derivative, halogenated analogs (chloro and fluoro) of N-substituted indole-2- and 3-carboxamide derivative, 2-(4-hydroxyphenyl)-3-phenylindole, indole carboxamides, indole acetamides, 3-benzolyl-1-methyl-4-phenyl-4-piperidinol, benzoyl phenyl benzoate derivative, 1-arginine derivative, guanidium HCL, L-NAME, HCN, linamarin, amygdalin, hederagenin, aescin, CIS-hinokiresinol and 1,3-di-p-hydroxyphenyl-4-penten-1-one.

For example, hyaluronan (HA) is included in the co-formulations provided herein that are stable at stress conditions of elevated temperatures of 32° C. to 40° C. for at least 3 days. Since HA oligomers are the substrate/product of the enzymatic reaction of a hyaluronan-degrading enzyme with hyaluronan, the hyaluronan oligomers can bind to the enzyme active site and cause the stabilizing effect. In examples herein, stable co-formulations contain hyaluronan (hyaluronic acid; HA) that has a molecular weight of 5 kDa to 5,000 kDa, 5 kDa to or to about 1,000 kDa, 5 kDa to or to about 200 kDa, or 5 kDa to or to about 50 kDa. In particular, the molecular weight of HA is less than 10 kDa. The HA can be an oligosaccharide, composed of disaccharides, such as a 2mer to 30mer or a 4mer to 16mer. The co-formulations of insulin and a hyaluronan-degrading enzyme such as a hyaluronidase, for example, a PH20 (e.g. rHuPH20) contain HA at a concentration of between or about between 1 mg/mL to 20 mg/mL, such as at least or about 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL or 20 mg/mL or more HA. Exemplary stable co-formulations include from or from about 8 mg/mL to or to about 12 mg/mL HA, such as, for example 10 mg/mL or about 10 mg/mL. In some examples, the molar ratio of HA to hyaluronan degrading enzyme is or is about 100,000:1, 95,000:1, 90,000:1, 85,000:1, 80,000:1, 75,000:1, 70,000:1, 65,000:1, 60,000:1, 55,000:1, 50,000:1, 45,000:1, 40,000:1, 35,000:1, 30,000:1, 25,000:1, 20,000:1, 15,000:1, 10,000:1, 5,000:1, 1,000:1, 900:1, 800:1, 700:1, 600:1, 500:1, 400:1, 300:1, 200:1, or 100:1 or less.

Nevertheless, it is also found that over time under stress conditions of elevated temperatures of 32° C. to 40° C., such as greater than 1 week or 2 weeks at 37° C., the presence of a hyaluronidase inhibitor, such as HA, in the co-formulation can result in degradation of insulin, thereby resulting in covalent HA-insulin analog adducts. For example, the presence of high concentrations of HA in the co-formulations provided herein has been shown by reverse-phase high performance liquid chromatography (RP-HPLC) to cause degradation of insulin Aspart® after 1 week at 37° C. and insulin Glulisine® after 2 weeks at 30° C. Liquid chromatography-mass spectrometry (LC-MS) analysis indicated that some of the degradation products are covalent HA-insulin analog glycation adducts formed by reaction of insulin with the reducing end of the HA. For example, one peak was determined to be the product of insulin Aspart® and a HA 7mer while another peak was the product of insulin Aspart® and a HA 2mer.

The presence of a hyaluronidase inhibitor, such as HA, also can have effects on the precipitation and color change of the co-formulation. Hence, while HA improves the stability of hyaluronan-degrading enzyme at stress conditions of elevated temperatures of 32° C. to 40° C., it also can have effects on insulin degradation, precipitation and color change of the co-formulation. It is within the level of one of skill in the art to monitor these conditions within desired safety and pharmacologic parameters and guidelines. Generally, stable co-formulations provided herein that contain a hyaluronidase inhibitor, such as HA, are stable at elevated temperatures, such as under stress conditions of temperatures of 32° C. to 40° C. for at least 3 hours but no more than 7 days due to effects on these parameters.

In some examples provided herein, a hyaluronidase inhibitor is used that is not capable of forming covalent complexes with insulin or a hyaluronan-degrading enzymes. Hence, non-covalent inhibitors that act by associative binding are contemplated in the formulations herein. For example, stable co-formulations contain HA with a reacted reducing end so that it is no longer possible to form glycation adducts with insulin. For example, in some examples, the HA used in the co-formulations provided herein has been modified by reductive amination. Reductive amination involves formation of a Schiff base between an aldehyde and amine, which is then reduced to form the more stable amine. The reducing end of a sugar, i.e., HA, exists as an equilibrium mixture of the cyclic hemiacetal form and the open chain aldehyde form. Under suitable conditions known of one of skill in the art, amine groups will condense with the sugar aldehyde to form an iminium ion which can be reduced to an amine, with a reducing agent such as sodium cyanoborohydride (see, e.g., Gildersleeve et al., (2008) Bioconjug Chem 19(7):1485-1490). The resulting HA is unreactive to the insulin and unable to form insulin glycation adducts.

c. Buffer

Any buffer can be used in co-formulations provided herein so long as it does not adversely affect the stability of the co-formulation, and supports the requisite pH range required. Examples of particularly suitable buffers include Tris, succinate, acetate, phosphate buffers, citrate, aconitate, malate and carbonate. Those of skill in the art, however, will recognize that formulations provided herein are not limited to a particular buffer, so long as the buffer provides an acceptable degree of pH stability, or “buffer capacity” in the range indicated. Generally, a buffer has an adequate buffer capacity within about 1 pH unit of its pK (Lachman et al., 1986). Buffer suitability can be estimated based on published pK tabulations or can be determined empirically by methods well known in the art. The pH of the solution can be adjusted to the desired endpoint within the range as described above, for example, using any acceptable acid or base.

Buffers that can be included in the co-formulations provided herein include, but are not limited to, Tris (Tromethamine), histidine, phosphate buffers, such as dibasic sodium phosphate, and citrate buffers. Generally, the buffering agent is present in an amount herein to maintain the pH range of the co-formulation between or about between 7.0 to 7.6. Such buffering agents can be present in the co-formulations at concentrations between or about between 1 mM to 100 mM, such as 10 mM to 50 mM or 20 mM to 40 mM, such as at or about 30 mM. For example, such buffering agents can be present in the co-formulations in a concentration of or about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, 50 mM, 55 mM, 60 mM, 65 mM, 70 mM, 75 mM, or more.

Exemplary of the buffers in the co-formulations herein are non-metal binding buffers such as Tris, which reduce insulin precipitation compared to metal-binding buffers, such as phosphate buffers. The inclusion of Tris as a buffer in the co-formulations provided herein has additional benefits. For example, the pH of a solution that is buffered with Tris is affected by the temperature at which the solution is held. Thus, when the insulin and hyaluronan-degrading enzyme co-formulations are prepared at room temperature at pH 7.3, upon refrigeration, the pH increases to approximately pH 7.6. Such a pH promotes insulin solubility at a temperature where insulin is otherwise likely to be insoluble. Conversely, at increased temperatures, the pH of the formulation decreases to approximately pH 7.1, which promotes hyaluronan-degrading enzyme stability at a temperature at which the enzyme is otherwise likely to become unstable. Thus, the solubility and stability of insulin and a hyaluronan-degrading enzyme, such as a hyaluronidase for example PH20 (e.g. rHuPH20) is maximized when the co-formulations contains Tris as a buffer compared to other buffers. Further, because Tris is a positive ion, the addition of NaCl into the solution as a counterion is not required. This also is beneficial to the overall stability of the co-formulation because NaCl at high concentrations is detrimental to insulin solubility.

Typically, Tris is included in the co-formulations provided herein at a concentration of or about 10 mM to 50 mM, such as, for example, 10 mM, 15 mM, 20 mM, 25 mM, 30 mM, 35 mM, 40 mM, 45 mM or 50 mM. In particular examples, the co-formulations contain or contain about 20 mM to 30 mM Tris, such as 21 mM, 22 mM, 23 mM, 24 mM, 25 mM, 26 mM, 27 mM, 28 mM, 29 mM or 30 mM Tris. In particular examples, the co-formulations provided herein contain Tris at a concentration of or about 30 mM.

d. Preservatives

Preservatives can have a deleterious effect on the solubility of insulin and the stability and activity of hyaluronan degrading enzymes, such as a PH20 (e.g. rHuPH20), while at the same time stabilizing the hexameric insulin molecules and being necessary as an anti-microbial agent in multidose formulations. Thus, the one or more preservatives present in the co-formulation cannot substantially destabilize the hyaluronan degrading enzyme, such as a hyaluronidase for example a PH20 (e.g. rHuPH20), so that it loses its activity over storage conditions (e.g. over time and at varied temperature). Further, these preservatives must be present in a sufficient concentration to stabilize the insulin hexamers and exert the required anti-microbial effect, but not be so concentrated as to decrease solubility of the insulin. Importantly, the preservatives must be present in a sufficient concentration to provide the anti-microbial requirements of, for example, the United States Pharmacopoeia (USP) and the European Pharmacopoeia (EP). Typically, formulations that meet EP (EPA or EPB) anti-microbial requirements contain more preservative than those formulated only to meet USP anti-microbial requirements.

Hence, the stable co-formulations contain preservative(s) in an amount that exhibits anti-microbial activity by killing or inhibiting the propagation of microbial organisms in a sample of the composition as assessed in an antimicrobial preservative effectiveness test (APET). One of skill in the art is familiar with the antimicrobial preservative effectiveness test and standards to be meet under the USP and EPA or EPB in order to meet minimum requirements. In general, the antimicrobial preservative effectiveness test involves challenging a composition, e.g., a co-formulation provided herein, with prescribed inoculums of suitable microorganisms, i.e., bacteria, yeast and fungi, storing the inoculated preparation at a prescribed temperature, withdrawing samples at specified intervals of time and counting the organisms in the sample (see, Sutton and Porter, (2002) PDA Journal of Pharmaceutical Science and Technology 56(11); 300-311; The United States Pharmacopeial Convention, Inc., (effective Jan. 1, 2002), The United States Pharmacopeia 25^{th} Revision, Rockville, Md., Chapter <51> Antimicrobial Effectiveness Testing; and European Pharmacopoeia, Chapter 5.1.3, Efficacy of Antimicrobial Preservation). The microorganisms used in the challenge generally include three strains of bacteria, namely E. coli (ATCC No. 8739), Pseudomonas aeruginosa (ATCC No. 9027) and Staphylococcus aureus (ATCC No. 6538), yeast (Candida albicans ATCC No. 10231) and fungus (Aspergillus niger ATCC No. 16404), all of which are added such that the inoculated composition contains 10⁵ or 10⁶ colony forming units (cfu) of microorganism per mL of composition. The preservative properties of the composition are deemed adequate if, under the conditions of the test, there is a significant fall or no increase, as specified in Table 5, below, in the number of microorganisms in the inoculated composition after the times and at the temperatures prescribed. The criteria for evaluation are given in terms of the log reduction in the number of viable microorganism as compared to the initial sample or the previous timepoint.

TABLE 5
USP and EP requirements for antimicrobial effectiveness testing

| | Criteria for passage |
|---|---|
| USP | |
| Bac- | Not less than 1.0 log reduction from the initial calculated count |
| teria | at 7 days, not less than 3.0 log reduction from the initial count |
| | at 14 days, and no increase from the 14 days count at 28 days. |
| | No increase is defined as not more than 0.5 log₁₀ unit higher |
| | than the previous measured value. |
| Yeast | No increase from the initial calculated count at 7, 14 and 28 |
| or | days. No increase is defined as not more than 0.5 log₁₀ unit |
| mold | higher than the previous measured value. |
| EPA | |
| Bac- | 2 log reduction in the number of viable microorganisms against |
| teria | the value obtained for the inoculum at 6 hours, a 3 log reduction |
| | in the number of viable microorganisms against the value |
| | obtained for the inoculum at 24 hours and no recovery at 28 days. |
| Yeast | 2 log reduction in the number of viable microorganisms against |
| or | the value obtained for the inoculum at 7 days and no increase at |
| mold | 28 days. No increase is defined as not more than 0.5 log₁₀ unit |
| | higher than the previous measured value. |
| EPB | |
| Bac- | 1 log reduction in the number of viable microorganisms against |
| teria | the value obtained for the inoculum at 24 hours, a 3 log reduction |
| | in the number of viable microorganisms against the value obtained |
| | for the inoculum at 7 days and no increase at 28 days. No increase |
| | is defined as not more than 0.5 log₁₀ unit higher than the previous |
| | measured value. |
| Yeast | 1 log reduction in the number of viable microorganisms against |
| or | the value obtained for the inoculum at 7 days and no increase at |
| mold | 28 days. No increase is defined as not more that 0.5 log₁₀ unit |
| | higher than the previous measured value. |

Specifically, the composition, for example, the co-formulation, is aliquoted into at least 5 containers, one each for each of the bacteria or fungi (Escherichia coli (ATCC No. 8739), Pseudomonas aeruginosa (ATCC No. 9027), Staphylococcus aureus (ATCC No. 6538), Candida albicans (ATCC No. 10231) and Aspergillus niger (ATCC No. 16404)). Each container is then inoculated with one of the test organisms to give an inoculum of 10⁵ or 10⁶ microorganisms per mL of the composition, with the inoculum not exceeding 1% of the volume of the composition. The inoculated compositions are maintained at a temperature between 20 and 25° C. for a period of 28 days, and samples removed at 6 hours, 24 hours, 7 days, 14 days and 28 days, depending upon the criteria set forth in Table 5 above. The number of viable microorganisms (cfu) in each sample is determined by plate count or membrane filtration. Finally, the cfu for each sample is compared to either the inoculum or the previous sample and log reduction is determined.

Under USP standards, the rate or level of the anti-microbial activity of preservatives in samples inoculated with the microbial organisms is at least a 1.0 log₁₀ unit reduction of bacterial organisms at 7 days following inoculation; at least a 3.0 log₁₀ unit reduction of bacterial organisms at 14 days following inoculation; and at least no further increase, i.e., not more than a 0.5 log₁₀ unit increase, in bacterial organisms from day 14 to day 28 following inoculation of the composition with the microbial inoculum. For fungal organisms according to USP standards, the rate or level of the anti-microbial activity of preservatives in samples inoculated with the microbial organisms is at least no increase from the initial amount after 7, 14 and 28 days following inoculation of the composition with the microbial inoculum. Under EPB, or minimum EP standards, the rate or level of the anti-microbial activity of preservatives in samples inoculated with the microbial organisms is at least 1 log₁₀ unit reduction of bacterial organisms at 24 hours following inoculation; at least a 3 log₁₀ unit reduction of bacterial organisms at 7 days following inoculation; and at least no further increase, i.e., not more than a 0.5 log₁₀ unit increase, in bacterial organisms 28 days following inoculation of the composition with the microbial inoculum. EPA standards require at least a 2 log₁₀ unit reduction of bacterial organisms at 6 hours following inoculation, with at least a 3 log₁₀ unit reduction of bacterial organisms at 24 hours following inoculation, and no recovery of microbial organisms 28 days after inoculation. For fungal organisms according to minimum EPB standards, the rate or level of the anti-microbial activity of preservatives in samples inoculated with the microbial organisms is at least 1 log₁₀ unit reduction of fungal organisms at 14 days following inoculation and no increase in fungal organisms at 28 days following inoculation of the composition, and increased EPA standards require a 2 log₁₀ unit reduction at 7 days following inoculation and no increase in fungal organisms at 28 days following inoculation of the composition.

Non-limiting examples of preservatives that can be included in the co-formulations provided herein include, but are not limited to, phenol, meta-cresol (m-cresol), methylparaben, benzyl alcohol, thimerosal, benzalkonium chloride, 4-chloro-1-butanol, chlorhexidine dihydrochloride, chlorhexidine digluconate, L-phenylalanine, EDTA, bronopol (2-bromo-2-nitropropane-1,3-diol), phenylmercuric acetate, glycerol (glycerin), imidurea, chlorhexidine, sodium dehydroacetate, ortho-cresol (o-cresol), para-cresol (p-cresol), chlorocresol, cetrimide, benzethonium chloride, ethylparaben, propylparaben or butylparaben and any combination thereof. For example, co-formulations provided herein can contain a single preservative. In other examples, the co-formulations contain at least two different preservatives or at least three different preservatives. For example, co-formulations provided herein can contain two preservatives such as L-phenylalanine and m-cresol, L-phenylalanine and methylparaben, L-phenylalanine and phenol, m-cresol and methylparaben, phenol and methylparaben, m-cresol and phenol or other similar combinations. In one example, the preservative in the co-formulation contains at least one phenolic preservative. For example, the co-formulation contains phenol, m-cresol or phenol and m-cresol.

In the co-formulations provided herein, the total amount of the one or more preservative agents as a percentage (%) of mass concentration (w/v) in the formulation can be, for example, between from or between about from 0.1% to 0.4%, such as 0.1% to 0.3%, 0.15% to 0.325%, 0.15% to 0.25%, 0.1% to 0.2%, 0.2% to 0.3%, or 0.3% to 0.4%. Generally, the co-formulations contain less than 0.4% (w/v) preservative. For example, the co-formulations provided herein contain at least or about at least 0.1%, 0.12%, 0.125%, 0.13%, 0.14%, 0.15%, 0.16% 0.17%, 0.175%, 0.18%, 0.19%, 0.2%, 0.25%, 0.3%, 0.325%, 0.35% but less than 0.4% total preservative.

In some examples, the stable co-formulations provided herein contain between or between about 0.1% to 0.25% phenol, and between or about between 0.05% to 0.2% m-cresol, such as between or about between 0.10% to 0.2% phenol and between or about between 0.6% to 0.18% m-cresol or between or about between 0.1% to 0.15% phenol and between or about between 0.8% to 0.15% m-cresol. For example, stable co-formulations provided herein contain or contain about 0.1% phenol and 0.075% m-cresol; 0.1% phenol and 0.15% m-cresol; 0.125% phenol and 0.075% m-cresol; 0.13% phenol and 0.075% m-cresol; 0.13% phenol and 0.08% m-cresol; 0.15% phenol and 0.175% m-cresol; or 0.17% phenol and 0.13% m-cresol.

e. Stabilizers

Included among the types of stabilizers that can be contained in the formulations provided herein are amino acids, amino acid derivatives, amines, sugars, polyols, salts and buffers, surfactants, and other agents. The co-formulations provided herein contain at least one stabilizer. For example, the co-formulations provided herein contain at least one, two, three, four, five, six or more stabilizers. Hence, any one or more of an amino acids, amino acid derivatives, amines, sugars, polyols, salts and buffers, surfactants, and other agents can be included in the co-formulations herein. Generally, the co-formulations herein contain at least contain a surfactant and an appropriate buffer. Optionally, the co-formulations provided herein can contain other additional stabilizers.

Exemplary amino acid stabilizers, amino acid derivatives or amines include, but are not limited to, L-Arginine, Glutamine, glycine, Lysine, Methionine, Proline, Lys-Lys, Gly-Gly, Trimethylamine oxide (TMAO) or betaine. Exemplary of sugars and polyols include, but are not limited to, glycerol, sorbitol, mannitol, inositol, sucrose or trehalose. Exemplary of salts and buffers include, but are not limited to, magnesium chloride, sodium sulfate, Tris such as Tris (100 mM), or sodium Benzoate. Exemplary surfactants include, but are not limited to, poloxamer 188 (e.g. Pluronic® F68), polysorbate 80 (PS80), polysorbate 20 (PS20). Other preservatives include, but are not limited to, hyaluronic acid (HA), human serum albumin (HSA), phenyl butyric acid, taurocholic acid, polyvinylpyrolidone (PVP) or zinc.

i. Surfactant

In some examples, the stable co-formulations contain one or more surfactants. Such surfactants inhibit aggregation of the hyaluronan-degrading enzyme, such as a hyaluronidase for example a PH20 (e.g. rHuPH20) and minimize absorptive loss. The surfactants generally are non-ionic surfactants. Surfactants that can be included in the co-formulations herein include, but are not limited to, partial and fatty acid esters and ethers of polyhydric alcohols such as of glycerol, or sorbitol, poloxamers and polysorbates. For example, exemplary surfactants in the co-formulations herein include any one or more of poloxamer 188 (PLURONICS® such as PLURONIC®F68), TETRONICS®, polysorbate 20, polysorbate 80, PEG 400, PEG 3000, Tween® (e.g. Tween® 20 or Tween® 80), Triton® X-100, SPAN®, MYRJ®, BRIJ®, CREMOPHOR®, polypropylene glycols or polyethylene glycols. In some examples, the co-formulations herein contain poloxamer 188, polysorbate 20, polysorbate 80, generally poloxamer 188 (pluronic F68). The co-formulations provided herein generally contain at least one surfactant, such as 1, 2 or 3 surfactants.

In the stable co-formulations, the total amount of the one or more surfactants as a percentage (%) of mass concentration (w/v) in the formulation can be, for example, between from or between about from 0.005% to 1.0%, such as between from or between about from 0.01% to 0.5%, such as 0.01% to 0.1% or 0.01% to 0.02%. Generally, the co-formulations contain at least 0.01% surfactant and contain less than 1.0%, such as less than 0.5% or less than 0.1% surfactant. For example, the co-formulations provided herein can contain at or about 0.001%, 0.005%, 0.01%, 0.015%, 0.02%, 0.025%, 0.03%, 0.035%, 0.04%, 0.045%, 0.05%, 0.055%, 0.06%, 0.065%, 0.07%, 0.08%, or 0.09%. In particular examples, the co-formulations provided herein contain or contain about 0.01% to or to about 0.05% surfactant.

Oxidation of the enzyme can be increased with increasing levels of surfactant. Also, the surfactant poloxamer 188 causes less oxidation than the polysorbates. Hence, the co-formulations herein generally contain poloxamer 188. Thus, although surfactants are able to stabilize a hyaluronan-degrading enzyme, the inclusion of surfactants in the co-formulations provided herein can result in oxidation of the hyaluronan-degrading enzyme at high concentrations. Thus, generally lower concentrations of surfactant are used in the co-formulations herein, for example, as a percentage (%) of mass concentration (w/v) of less than 1.0% and generally between or about between 0.01% or 0.05%. Also, as provided herein below, optionally an anti-oxidation agent can be included in the formulation to reduce or prevent oxidation.

Exemplary co-formulations provided herein contain poloxamer 188. Poloxamer 188 has a higher critical micelle concentration (cmc). Thus, use of poloxamer 188 can reduce the formation of micelles in the formulation, which can in turn reduce the effectiveness of the preservatives. Thus, among the co-formulations provided herein are those that contain or contain about 0.01% or 0.05% poloxamer 188.

ii. Other Stabilizers

The stable co-formulations optionally can contain other components that, when combined with the preservatives, salt and stabilizers at the appropriate pH, as discussed above, result in a stable co-formulation. Other components include, for example, one or more tonicity modifiers, one or more anti-oxidation agents, zinc or other stabilizer.

For example, tonicity modifiers can be included in the formulation to produce a solution with the desired osmolarity. The stable co-formulations have an osmolarity of between or about between 245 mOsm/kg to 305 mOsm/kg. For example, the osmolarity is or is about 245 mOsm/kg, 250 mOsm/kg, 255 mOsm/kg, 260 mOsm/kg, 265 mOsm/kg, 270 mOsm/kg, 275 mOsm/kg, 280 mOsm/kg, 285 mOsm/kg, 290 mOsm/kg, 295 mOsm/kg, 300 mOsm/kg or 305 mOsm/kg. In some examples, the co-formulations of an insulin and a hyaluronan-degrading enzyme, such as a hyaluronidase for example a PH20 (e.g. rHuPH20) have an osmolarity of or of about 275 mOsm/kg.

Tonicity modifiers include, but are not limited to, glycerin, NaCl, amino acids, polyalcohols, trehalose, and other salts and/or sugars. In other instances, glycerin (glycerol) is included in the co-formulations. For example, co-formulations provided herein typically contain less than 60 mM glycerin, such as less than 55 mM, less than 50 mM, less than 45 mM, less than 40 mM, less than 35 mM, less than 30 mM, less than 25 mM, less than 20 mM, less than 15 mM, 10 mM or less. The amount of glycerin typically depends on the amount of NaCl present: the more NaCl present in the co-formulation, the less glycerin is required to achieve the desired osmolarity. Thus, for example, in co-formulations containing higher NaCl concentrations, such as those formulated with insulins with higher apparent solubility (e.g. insulin glulisine), little or no glycerin need be included in the formulation. In contrast, in co-formulations containing slightly lower NaCl concentrations, such as those formulated with insulins with lower apparent solubility (e.g. insulin aspart), glycerin can be included. For example, co-formulations contain insulin aspart contain glycerin at a concentration less than 50 mM, such as 20 mM to 50 mM, for example at or about 50 mM. In co-formulations containing an even lower NaCl concentration, such as those formulated with insulins with the lowest apparent solubility (e.g. insulin lispro or regular insulin), glycerin is included at a concentration of or of about, for example, 40 mM to 60 mM.

The co-formulations also can contain antioxidants to reduce or prevent oxidation, in particular oxidation of the hyaluronan-degrading enzyme. Exemplary antioxidants include, but are not limited to, cysteine, tryptophan and methionine. In particular examples, the anti-oxidant is methionine. The co-formulations provided herein containing an insulin and a hyaluronan-degrading enzyme, such as a hyaluronidase for example a PH20 (e.g. rHuPH20) can include an antioxidant at a concentration from between or from about between 5 mM to or to about 50 mM, such as 5 mM to 40 mM, 5 mM to 20 mM or 10 mM to 20 mM. For example, methionine can be provided in the co-formulations herein at a concentration from between or from about between 5 mM to or to about 50 mM, such as 5 mM to 40 mM, 5 mM to 20 mM or 10 mM to 20 mM. For example, an antioxidant, for example methionine, can be included at a concentration that is or is about 5 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 35 mM, 40 mM, 45 mM or 50 mM. In some examples, the co-formulations contain 10 mM to 20 mM methionine, such as or about 10 mM or 20 mM methionine.

In some instances, zinc is included in the co-formulations as a stabilizer for insulin hexamers. For example, formulations containing regular insulin, insulin lispro or insulin aspart typically contain zinc, whereas formulations containing insulin glulisine do not contain zinc. Zinc can be provided, for example, as zinc oxide, zinc acetate or zinc chloride. Zinc can be present in a composition provided herein at between or about between 0.001 to 0.1 mg per 100 units of insulin (mg/100 U), 0.001 to 0.05 mg per 100 U or 0.01 to 05 mg per 100 U. For example, the co-formulations provided herein can contain zinc at or about 0.002 milligrams per 100 units of insulin (mg/100 U), 0.005 mg/100 U, 0.01 mg/100 U, 0.012 mg/100 U, 0.014 mg/100 U, 0.016 mg/100 U, 0.017 mg/100 U, 0.018 mg/100 U, 0.02 mg/100 U, 0.022 mg/100 U, 0.024 mg/100 U, 0.026 mg/100 U, 0.028 mg/100 U, 0.03 mg/100 U, 0.04 mg/100 U, 0.05 mg/100 U, 0.06 mg/100 U, 0.07 mg/100 U, 0.08 mg/100 U or 0.1 mg/100 U.

The stable co-formulation also can contain an amino acid stabilizer, which contributes to the stability of the preparation. The stabilizer can be a non-polar and basic amino acids. Exemplary non-polar and basic amino acids include, but are not limited to, alanine, histidine, arginine, lysine, ornithine, isoleucine, valine, methionine, glycine and proline. For example, the amino acid stabilizer is glycine or proline, typically glycine. The stabilizer can be a single amino acid or it can be a combination of 2 or more such amino acids. The amino acid stabilizers can be natural amino acids, amino acid analogues, modified amino acids or amino acid equivalents. Generally, the amino acid is an L-amino acid. For example, when proline is used as the stabilizer, it is generally L-proline. It is also possible to use amino acid equivalents, for example, proline analogues. The concentration of amino acid stabilizer, for example glycine, included in the co-formulation ranges from 0.1 M to 1 M amino acid, typically 0.1 M to 0.75 M, generally 0.2 M to 0.5 M, for example, at least at or about 0.1 M, 0.15 M, 0.2 M, 0.25 M, 0.3 M, 0.35 M, 0.4 M, 0.45 M, 0.5 M, 0.6 M, 0.7 M, 0.75 M or more. The amino acid, for example glycine, can be used in a form of a pharmaceutically acceptable salt, such as hydrochloride, hydrobromide, sulfate, acetate, etc. The purity of the amino acid, for example glycine, should be at least 98%, at least 99%, or at least 99.5% or more.

2. Other Excipients or Agents

Optionally, the stable co-formulations can include carriers such as a diluent, adjuvant, excipient, or vehicle with which the co-formulation is administered. Examples of suitable pharmaceutical carriers are described in “Remington's Pharmaceutical Sciences” by E. W. Martin. Such compositions will contain a therapeutically effective amount of the compound, generally in purified form or partially purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions.

For example, pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers, which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acacia, gelatin, glucose, molasses, polyvinylpyrrolidone, celluloses and derivatives thereof, povidone, crospovidones and other such binders known to those of skill in the art.

For example, an excipient protein can be added to the co-formulation that can be any of a number of pharmaceutically acceptable proteins or peptides. Generally, the excipient protein is selected for its ability to be administered to a mammalian subject without provoking an immune response. For example, human serum albumin is well-suited for use in pharmaceutical formulations. Other known pharmaceutical protein excipients include, but are not limited to, starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. The excipient is included in the formulation at a sufficient concentration to prevent adsorption of the protein to the holding vessel or vial. The concentration of the excipient will vary according to the nature of the excipient and the concentration of the protein in the co-formulation.

A composition, if desired, also can contain minor amounts of wetting or emulsifying agents, or pH buffering agents, for example, acetate, sodium citrate, cyclodextrin derivatives, sorbitan monolaurate, triethanolamine sodium acetate, triethanolamine oleate, and other such agents.

### G. METHODS OF PRODUCING NUCLEIC ACIDS ENCODING AN INSULIN OR HYALURONAN DEGRADING ENZYME AND POLYPEPTIDES THEREOF

Polypeptides of an insulin and hyaluronan degrading enzyme set forth herein can be obtained by methods well known in the art for protein purification and recombinant protein expression. Polypeptides also can be synthesized chemically. For example, the A-chain and B-chain of insulin can be chemically synthesized and then cross-linked by disulfide bonds through, for example, a reduction-reoxidation reaction. When the polypeptides are produced by recombinant means, any method known to those of skill in the art for identification of nucleic acids that encode desired genes can be used. Any method available in the art can be used to obtain a full length (i.e., encompassing the entire coding region) cDNA or genomic DNA clone encoding a hyaluronidase, such as from a cell or tissue source. Modified or variant insulins or hyaluronan degrading enzymes can be engineered from a wildtype polypeptide, such as by site-directed mutagenesis.

Polypeptides can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

Methods for amplification of nucleic acids can be used to isolate nucleic acid molecules encoding a desired polypeptide, including for example, polymerase chain reaction (PCR) methods. A nucleic acid containing material can be used as a starting material from which a desired polypeptide-encoding nucleic acid molecule can be isolated. For example, DNA and mRNA preparations, cell extracts, tissue extracts, fluid samples (e.g. blood, serum, saliva), and samples from healthy and/or diseased subjects can be used in amplification methods. Nucleic acid libraries also can be used as a source of starting material. Primers can be designed to amplify a desired polypeptide. For example, primers can be designed based on expressed sequences from which a desired polypeptide is generated. Primers can be designed based on back-translation of a polypeptide amino acid sequence. Nucleic acid molecules generated by amplification can be sequenced and confirmed to encode a desired polypeptide.

Additional nucleotide sequences can be joined to a polypeptide-encoding nucleic acid molecule, including linker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to a polypeptide-encoding nucleic acid molecule. Examples of such sequences include, but are not limited to, promoter sequences designed to facilitate intracellular protein expression, and secretion sequences, for example heterologous signal sequences, designed to facilitate protein secretion. Such sequences are known to those of skill in the art. Additional nucleotide residues sequences such as sequences of bases specifying protein binding regions also can be linked to enzyme-encoding nucleic acid molecules. Such regions include, but are not limited to, sequences of residues that facilitate or encode proteins that facilitate uptake of an enzyme into specific target cells, or otherwise alter pharmacokinetics of a product of a synthetic gene. For example, enzymes can be linked to PEG moieties.

In addition, tags or other moieties can be added, for example, to aid in detection or affinity purification of the polypeptide. For example, additional nucleotide residues sequences such as sequences of bases specifying an epitope tag or other detectable marker also can be linked to enzyme-encoding nucleic acid molecules. Exemplary of such sequences include nucleic acid sequences encoding a His tag (e.g., 6×His, HHHHHH; SEQ ID NO:54) or Flag Tag (DYKDDDDK; SEQ ID NO:55).

The identified and isolated nucleic acids can then be inserted into an appropriate cloning vector. A large number of vector-host systems known in the art can be used. Possible vectors include, but are not limited to, plasmids or modified viruses, but the vector system must be compatible with the host cell used. Such vectors include, but are not limited to, bacteriophages such as lambda derivatives, or plasmids such as pCMV4, pBR322 or pUC plasmid derivatives or the Bluescript vector (Stratagene, La Jolla, Calif.). Other expression vectors include the HZ24 expression vector exemplified herein. The insertion into a cloning vector can, for example, be accomplished by ligating the DNA fragment into a cloning vector which has complementary cohesive termini. Insertion can be effected using TOPO cloning vectors (Invitrogen, Carlsbad, Calif.). If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized oligonucleotides encoding restriction endonuclease recognition sequences. In an alternative method, the cleaved vector and protein gene can be modified by homopolymeric tailing. Recombinant molecules can be introduced into host cells via, for example, transformation, transfection, infection, electroporation and sonoporation, so that many copies of the gene sequence are generated.

Insulin can be produced using a variety of techniques (see e.g. Ladisch et al. (1992) Biotechnol. Prog. 8:469-478). In some examples, nucleic acid encoding a preproinsulin or proinsulin polypeptide is inserted into an expression vector. Upon expression, the preproinsulin or proinsulin polypeptide is converted to insulin by enzymatic or chemical methods that cleave the signal sequence and/or the C peptide, resulting in the A- and B-chains that are cross-linked by disulfide bonds through, for example, a reduction-reoxidation reaction (see e.g. Cousens et al., (1987) Gene 61:265-275, Chance et al., (1993) Diabetes Care 4:147-154). In another example, the nucleic acid encoding the A-chain and B-chain of an insulin are inserted into one or two expression vectors for co-expression as a single polypeptide from one expression vector or expression as two polypeptides from one or two expression vectors. Thus, the A- and B-chain polypeptides can be expressed separately and then combined to generate an insulin, or can be co-expressed, in the absence of a C chain. In instances where the A- and B-chains are co-expressed as a single polypeptide, the nucleic acid encoding the subunits also can encode a linker or spacer between the B-chain and A-chain, such as a linker or spacer described below. The nucleic acid inserted into the expression vector can contain, for example, nucleic acid encoding the insulin B-chain, a linker, such as for example, an alanine-alanine-lysine linker, and the A-chain, resulting in expression of, for example, “insulin B chain-Ala-Ala-Lys-insulin A chain.”

In specific embodiments, transformation of host cells with recombinant DNA molecules that incorporate the isolated protein gene, cDNA, or synthesized DNA sequence enables generation of multiple copies of the gene. Thus, the gene can be obtained in large quantities by growing transformants, isolating the recombinant DNA molecules from the transformants and, when necessary, retrieving the inserted gene from the isolated recombinant DNA.

1. Vectors and Cells

For recombinant expression of one or more of the desired proteins, such as any described herein, the nucleic acid containing all or a portion of the nucleotide sequence encoding the protein can be inserted into an appropriate expression vector, i.e., a vector that contains the necessary elements for the transcription and translation of the inserted protein coding sequence. The necessary transcriptional and translational signals also can be supplied by the native promoter for enzyme genes, and/or their flanking regions.

Also provided are vectors that contain a nucleic acid encoding the enzyme. Cells containing the vectors also are provided. The cells include eukaryotic and prokaryotic cells, and the vectors are any suitable vector for use therein.

Prokaryotic and eukaryotic cells, including endothelial cells, containing the vectors are provided. Such cells include bacterial cells, yeast cells, fungal cells, Archea, plant cells, insect cells and animal cells. The cells are used to produce a protein thereof by growing the above-described cells under conditions whereby the encoded protein is expressed by the cell, and recovering the expressed protein. For purposes herein, for example, the enzyme can be secreted into the medium.

Provided are vectors that contain a sequence of nucleotides that encodes the soluble hyaluronidase polypeptide coupled to the native or heterologous signal sequence, as well as multiple copies thereof. The vectors can be selected for expression of the enzyme protein in the cell or such that the enzyme protein is expressed as a secreted protein.

A variety of host-vector systems can be used to express the protein encoding sequence. These include but are not limited to mammalian cell systems infected with virus (e.g. vaccinia virus, adenovirus and other viruses); insect cell systems infected with virus (e.g. baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system used, any one of a number of suitable transcription and translation elements can be used.

Any methods known to those of skill in the art for the insertion of DNA fragments into a vector can be used to construct expression vectors containing a chimeric gene containing appropriate transcriptional/translational control signals and protein coding sequences. These methods can include in vitro recombinant DNA and synthetic techniques and in vivo recombinants (genetic recombination). Expression of nucleic acid sequences encoding protein, or domains, derivatives, fragments or homologs thereof, can be regulated by a second nucleic acid sequence so that the genes or fragments thereof are expressed in a host transformed with the recombinant DNA molecule(s). For example, expression of the proteins can be controlled by any promoter/enhancer known in the art. In a specific embodiment, the promoter is not native to the genes for a desired protein. Promoters which can be used include but are not limited to the SV40 early promoter (Bernoist and Chambon, Nature 290:304-310 (1981)), the promoter contained in the 3′ long terminal repeat of Rous sarcoma virus (Yamamoto et al. Cell 22:787-797 (1980)), the herpes thymidine kinase promoter (Wagner et al., Proc. Natl. Acad. Sci. USA 78:1441-1445 (1981)), the regulatory sequences of the metallothionein gene (Brinster et al., Nature 296:39-42 (1982)); prokaryotic expression vectors such as the β-lactamase promoter (Jay et al., (1981) Proc. Natl. Acad. Sci. USA 78:5543) or the tac promoter (DeBoer et al., Proc. Natl. Acad. Sci. USA 80:21-25 (1983)); see also “Useful Proteins from Recombinant Bacteria”: in Scientific American 242:79-94 (1980)); plant expression vectors containing the nopaline synthetase promoter (Herrara-Estrella et al., Nature 303:209-213 (1984)) or the cauliflower mosaic virus 35S RNA promoter (Garder et al., Nucleic Acids Res. 9:2871 (1981)), and the promoter of the photosynthetic enzyme ribulose bisphosphate carboxylase (Herrera-Estrella et al., Nature 310:115-120 (1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 38:639-646 (1984); Ornitz et al., Cold Spring Harbor Symp. Quant. Biol. 50:399-409 (1986); MacDonald, Hepatology 7:425-515 (1987)); insulin gene control region which is active in pancreatic beta cells (Hanahan et al., Nature 315:115-122 (1985)), immunoglobulin gene control region which is active in lymphoid cells (Grosschedl et al., Cell 38:647-658 (1984); Adams et al., Nature 318:533-538 (1985); Alexander et al., Mol. Cell. Biol. 7:1436-1444 (1987)), mouse mammary tumor virus control region which is active in testicular, breast, lymphoid and mast cells (Leder et al., Cell 45:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-1648 (1985); Hammer et al., Science 235:53-58 1987)), alpha-1 antitrypsin gene control region which is active in liver (Kelsey et al., Genes and Devel. 1:161-171 (1987)), beta globin gene control region which is active in myeloid cells (Magram et al., Nature 315:338-340 (1985); Kollias et al., Cell 46:89-94 (1986)), myelin basic protein gene control region which is active in oligodendrocyte cells of the brain (Readhead et al., Cell 48:703-712 (1987)), myosin light chain-2 gene control region which is active in skeletal muscle (Shani, Nature 314:283-286 (1985)), and gonadotrophic releasing hormone gene control region which is active in gonadotrophs of the hypothalamus (Mason et al., Science 234:1372-1378 (1986)).

In a specific embodiment, a vector is used that contains a promoter operably linked to nucleic acids encoding a desired protein, or a domain, fragment, derivative or homolog, thereof, one or more origins of replication, and optionally, one or more selectable markers (e.g., an antibiotic resistance gene). Exemplary plasmid vectors for transformation of E. coli cells, include, for example, the pQE expression vectors (available from Qiagen, Valencia, Calif.; see also literature published by Qiagen describing the system). pQE vectors have a phage T5 promoter (recognized by E. coli RNA polymerase) and a double lac operator repression module to provide tightly regulated, high-level expression of recombinant proteins in E. coli, a synthetic ribosomal binding site (RBS II) for efficient translation, a 6×His tag coding sequence, t₀ and T1 transcriptional terminators, ColE1 origin of replication, and a beta-lactamase gene for conferring ampicillin resistance. The pQE vectors enable placement of a 6×His tag at either the N- or C-terminus of the recombinant protein. Such plasmids include pQE 32, pQE 30, and pQE 31 which provide multiple cloning sites for all three reading frames and provide for the expression of N-terminally 6×His-tagged proteins. Other exemplary plasmid vectors for transformation of E. coli cells include, for example, the pET expression vectors (see, U.S. Pat. No. 4,952,496; available from Novagen, Madison, Wis.; see, also literature published by Novagen describing the system). Such plasmids include pET 11a, which contains the T7-lac promoter, T7 terminator, the inducible E. coli lac operator, and the lac repressor gene; pET 12a-c, which contains the T7 promoter, T7 terminator, and the E. coli ompT secretion signal; and pET 15b and pET19b (Novagen, Madison, Wis.), which contain a His-Tag™ leader sequence for use in purification with a His column and a thrombin cleavage site that permits cleavage following purification over the column, the T7-lac promoter region and the T7 terminator.

Exemplary of a vector for mammalian cell expression is the HZ24 expression vector. The HZ24 expression vector was derived from the pCI vector backbone (Promega). It contains DNA encoding the Beta-lactamase resistance gene (AmpR), an F1 origin of replication, a Cytomegalovirus immediate-early enhancer/promoter region (CMV), and an SV40 late polyadenylation signal (SV40). The expression vector also has an internal ribosome entry site (IRES) from the ECMV virus (Clontech) and the mouse dihydrofolate reductase (DHFR) gene.

2. Linker Moieties

In some examples, insulin is prepared by generating the A-chain and B-chain polypeptides with a linker, such that, for example, the C-terminus of the B-chain is joined to the N-terminus of the A-chain by a short linker. The A-chain and B-chains can be expressed from a single polypeptide containing a linker, or can be expressed separately and then joined by a linker. The linker moiety is selected depending upon the properties desired. The linker moiety should be long enough and flexible enough to allow the A-chain and B-chain to mimic the natural conformation of the insulin.

Linkers can be any moiety suitable to the insulin A-chain and B-chain. Such moieties include, but are not limited to, peptidic linkages; amino acid and peptide linkages, typically containing between one and about 60 amino acids; chemical linkers, such as heterobifunctional cleavable cross-linkers, photocleavable linkers and acid cleavable linkers.

The linker moieties can be peptides. The peptide typically has from about 2 to about 60 amino acid residues, for example from about 5 to about 40, or from about 10 to about 30 amino acid residues. Peptidic linkers can conveniently be encoded by nucleic acid and incorporated in fusion proteins upon expression in a host cell, such as E. coli. In one example, an alanine-alanine-lysine (AAK) (SEQ ID NO:178) linker is encoded in a nucleic acid between nucleic acid encoding the insulin B-chain and nucleic acid encoding the A-chain, such that upon expression, an “insulin B-chain-AAK-insulin A chain” polypeptide is produced. Peptide linkers can be a flexible spacer amino acid sequence, such as those known in single-chain antibody research. Examples of such known linker moieties include, but are not limited to, RPPPPC (SEQ ID NO:166) or SSPPPPC (SEQ ID NO:167), GGGGS (SEQ ID NO:168), (GGGGS)ₙ (SEQ. ID NO:169), GKSSGSGSESKS (SEQ ID NO:170), GSTSGSGKSSEGKG (SEQ. ID NO:171), GSTSGSGKSSEGSGSTKG (SEQ ID NO:172), GSTSGSGKSSEGKG (SEQ ID NO:173), GSTSGSGKPGSGEGSTKG (SEQ ID NO:174), EGKSSGSGSESKEF (SEQ ID NO:175), SRSSG (SEQ. ID NO:176) and SGSSC (SEQ ID NO:177).

Alternatively, the peptide linker moiety can be VM (SEQ ID NO: 179) or AM (SEQ ID NO: 180), or have the structure described by the formula: AM(G_{2 to 4}S)ₓAM wherein X is an integer from 1 to 11 (SEQ ID NO: 181). Additional linking moieties are described, for example, in Huston et al. (1988) Proc. Natl. Acad. Sci. U.S.A. 85:5879-5883; Whitlow, M., et al. (1993) Protein Engineering 6:989-995; Newton et al. (1996) Biochemistry 35:545-553; A. J. Cumber et al. (1992) Bioconj. Chem. 3:397-401; Ladurner et al., (1997) J. Mol. Biol. 273:330-337; and U.S. Pat. No. 4,894,443.

In some examples, peptide linkers are encoded by nucleic acid and incorporated between the B-chain and A-chain upon expression in a host cell, such as E. coli or S. cerevisiae. In other examples, a peptide linker is synthesized by chemical methods. This can be performed in a separate protocol to the synthesis of one or more of the A- and B-chain, after which the components are joined, such as with the use of heterobifunctional linkers. Alternatively, a peptide linker can be synthesized at the N- or C-terminus of one of the insulin chains, which is then linked to the other chain via the peptide linker, such as with a heterobifunctional linker.

Any linker known to those of skill in the art can be used herein to link the insulin A-chain and B-chain. Linkers and linkages that are suitable for chemically linking the chains include, but are not limited to, disulfide bonds, thioether bonds, hindered disulfide bonds, and covalent bonds between free reactive groups, such as amine and thiol groups. These bonds are produced using heterobifunctional reagents to produce reactive thiol groups on one or both of the polypeptides and then reacting the thiol groups on one polypeptide with reactive thiol groups or amine groups to which reactive maleimido groups or thiol groups can be attached on the other. Other linkers include, acid cleavable linkers, such as bismaleimideothoxy propane, acid labile-transferrin conjugates and adipic acid dihydrazide, that would be cleaved in more acidic intracellular compartments; cross linkers that are cleaved upon exposure to UV or visible light and linkers, such as the various domains, such as CH1, CH2, and CH3, from the constant region of human IgG1 (see, Batra et al. (1993) Molecular Immunol. 30:379-386). In some embodiments, several linkers can be included in order to take advantage of desired properties of each linker. Chemical linkers and peptide linkers can be inserted by covalently coupling the linker to the insulin A-chain and B-chain. The heterobifunctional agents, described below, can be used to effect such covalent coupling. Peptide linkers also can be linked by expressing DNA encoding the linker between the B-chain and A-chain.

Other linkers that can be used to join the A-chain and B-chain of insulin include: enzyme substrates, such as cathepsin B substrate, cathepsin D substrate, trypsin substrate, thrombin substrate, subtilisin substrate, Factor Xa substrate, and enterokinase substrate; linkers that increase solubility, flexibility, and/or intracellular cleavability include linkers, such as (glyₘser)ₙ and (serₘgly)ₙ, in which m is 1 to 6, preferably 1 to 4, more preferably 2 to 4, and n is 1 to 30, preferably 1 to 10, more preferably 1 to 4 (see, e.g., International PCT application No. WO 96/06641, which provides exemplary linkers). In some embodiments, several linkers can be included in order to take advantage of desired properties of each linker.

3. Expression

Insulin and hyaluronan degrading enzyme polypeptides can be produced by any method known to those of skill in the art including in vivo and in vitro methods.

Desired proteins can be expressed in any organism suitable to produce the required amounts and forms of the proteins, such as for example, needed for administration and treatment. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

Many expression vectors are available and known to those of skill in the art and can be used for expression of proteins. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

Soluble hyaluronidase polypeptides also can be utilized or expressed as protein fusions. For example, an enzyme fusion can be generated to add additional functionality to an enzyme. Examples of enzyme fusion proteins include, but are not limited to, fusions of a signal sequence, a tag such as for localization, e.g. a his₆ tag or a myc tag, or a tag for purification, for example, a GST fusion, and a sequence for directing protein secretion and/or membrane association.

a. Prokaryotic Cells

Prokaryotes, especially E. coli, provide a system for producing large amounts of proteins. Transformation of E. coli is a simple and rapid technique well known to those of skill in the art. Expression vectors for E. coli can contain inducible promoters, which include promoters that are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated APL promoter.

Proteins, such as any provided herein, can be expressed in the cytoplasmic environment of E. coli. The cytoplasm is a reducing environment and for some molecules, this can result in the formation of insoluble inclusion bodies. Reducing agents such as dithiothreitol and β-mercaptoethanol and denaturants, such as guanidine-HCl and urea can be used to resolubilize the proteins. An alternative approach is the expression of proteins in the periplasmic space of bacteria, which contains an oxidizing environment and chaperonin-like and disulfide isomerase and can lead to the production of soluble protein. Typically, a leader sequence is fused to the protein to be expressed which directs the protein to the periplasm. The leader is then removed by signal peptidases inside the periplasm. Examples of periplasmic-targeting leader sequences include the pelB leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Typically, bacteria produce aglycosylated proteins. Thus, if proteins require glycosylation for function, glycosylation can be added in vitro after purification from host cells.

b. Yeast Cells

Yeasts such, as Saccharomyces cerevisae, Schizosaccharomyces pombe, Yarrowia hpolytica, Kluyveromyces lactis and Pichia pastoris are well known yeast expression hosts that can be used for production of proteins, such as any described herein. Yeast can be transformed with episomal replicating vectors or by stable chromosomal integration by homologous recombination. Typically, inducible promoters are used to regulate gene expression. Examples of such promoters include GAL1, GAL7 and GAL5 and metallothionein promoters, such as CUP1, AOX1 or other Pichia or other yeast promoter. Expression vectors often include a selectable marker such as LEU2, TRP1, HIS3 and URA3 for selection and maintenance of the transformed DNA. Proteins expressed in yeast are often soluble. Co-expression with chaperonins such as Bip and protein disulfide isomerase can improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site such as for the Kex-2 protease, can be engineered to remove the fused sequences from the expressed polypeptides as they exit the secretion pathway. Yeast also is capable of glycosylation at Asn-X-Ser/Thr motifs.

c. Insect Cells

Insect cells, particularly using baculovirus expression, are useful for expressing polypeptides such as hyaluronidase polypeptides. Insect cells express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter of baculovirus. Commonly used baculovirus systems include the baculoviruses such as Autographa californica nuclear polyhedrosis virus (AcNPV), and the Bombyx mori nuclear polyhedrosis virus (BmNPV) and an insect cell line such as Sf9 derived from Spodoptera frugiperda, Pseudaletia unipuncta (A7S) and Danaus plexippus (DpN1). For high-level expression, the nucleotide sequence of the molecule to be expressed is fused immediately downstream of the polyhedrin initiation codon of the virus.

Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schneider 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

d. Mammalian Cells

Mammalian expression systems can be used to express proteins including soluble hyaluronidase polypeptides. Expression constructs can be transferred to mammalian cells by viral infection such as adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. IRES elements also can be added to permit bicistronic expression with another gene, such as a selectable marker. Such vectors often include transcriptional promoter-enhancers for high-level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase (DHFR) and thymidine kinase. For example, expression can be performed in the presence of methotrexate to select for only those cells expressing the DHFR gene. Fusion with cell surface signaling molecules such as TCR-ζ and Fc_{ε}RI-γ can direct expression of the proteins in an active state on the cell surface.

Many cell lines are available for mammalian expression including mouse, rat human, monkey, chicken and hamster cells. Exemplary cell lines include but are not limited to CHO, Balb/3T3, HeLa, MT2, mouse NS0 (nonsecreting) and other myeloma cell lines, hybridoma and heterohybridoma cell lines, lymphocytes, fibroblasts, Sp2/0, COS, NIH3T3, HEK293, 293S, 2B8, and HKB cells. Cell lines also are available adapted to serum-free media which facilitates purification of secreted proteins from the cell culture media. Examples include CHO—S cells (Invitrogen, Carlsbad, Calif., cat #11619-012) and the serum free EBNA-1 cell line (Pham et al., (2003) Biotechnol. Bioeng. 84:332-42.). Cell lines also are available that are adapted to grow in special mediums optimized for maximal expression. For example, DG44 CHO cells are adapted to grow in suspension culture in a chemically defined, animal product-free medium.

e. Plants

Transgenic plant cells and plants can be used to express proteins such as any described herein. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline synthase promoter, the ribose bisphosphate carboxylase promoter and the ubiquitin and UBQ3 promoters. Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce hyaluronidase polypeptides. Because plants have different glycosylation patterns than mammalian cells, this can influence the choice of protein produced in these hosts.

4. Purification Techniques

Method for purification of polypeptides, including insulin and hyaluronan degrading enzyme polypeptides or other proteins, from host cells will depend on the chosen host cells and expression systems. For secreted molecules, proteins are generally purified from the culture media after removing the cells. For intracellular expression, cells can be lysed and the proteins purified from the extract. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary, the proteins can be extracted and further purified using standard methods in the art.

Proteins, such as insulin polypeptides or hyaluronan degrading enzyme polypeptides, can be purified using standard protein purification techniques known in the art including but not limited to, SDS-PAGE, size fractionation and size exclusion chromatography, ammonium sulfate precipitation and ionic exchange chromatography, such as anion exchange chromatography. Affinity purification techniques also can be utilized to improve the efficiency and purity of the preparations. For example, antibodies, receptors and other molecules that bind hyaluronidase enzymes can be used in affinity purification. Expression constructs also can be engineered to add an affinity tag to a protein such as a myc epitope, GST fusion or His₆ and affinity purified with myc antibody, glutathione resin and Ni-resin, respectively. Purity can be assessed by any method known in the art including gel electrophoresis, orthogonal HPLC methods, staining and spectrophotometric techniques.

### H. THERAPEUTIC USES

The CSII methods, including hyaluronan-degrading enzyme leading edge CSII methods, provided herein can be used for treatment of any condition for which a fast-acting insulin is employed. This section provides exemplary therapeutic uses of fast-acting insulin. The therapeutic uses described below are exemplary and do not limit the applications of the methods described herein. Therapeutic uses include, but are not limited to, treatment for type 1 diabetes mellitus, type 2 diabetes mellitus, gestational diabetes, and for glycemic control in critically ill patients. It is within the skill of a treating physician to identify such diseases or conditions.

As discussed above, particular dosages and treatment protocols are typically individualized for each subject. If necessary, a particular dosage and duration and treatment protocol can be empirically determined or extrapolated. For example, exemplary doses of fast-acting insulin without a hyaluronan degrading enzyme can be used as a starting point to determine appropriate dosages in the methods provided herein. Dosage levels can be determined based on a variety of factors, such as body weight of the individual, general health, age, the activity of the specific compound employed, sex, diet, metabolic activity, blood glucose concentrations, time of administration, rate of excretion, drug combination, the severity and course of the disease, and the patient's disposition to the disease and the judgment of the treating physician. In particular, blood glucose levels, such as measured by a blood glucose sensor, can be measured and used to determine the amount of insulin and a hyaluronan degrading enzyme to be administered to achieve glycemic control. Algorithms are known in the art that can be used to determine a dose based on the rate of absorption and level of absorption of the co-formulations of a fast acting insulin and a hyaluronan degrading enzyme provided herein, and also based upon blood glucose levels. Dosages of insulin for post-prandial glycemic control also can be calculated or adjusted, for example, by determining the carbohydrate content of a meal (see, e.g., Bergenstal et al., (2008) Diabetes Care 31:1305-1310, Lowe et al., (2008) Diabetes Res. Clin. Pract. 80:439-443, Chiesa et a/42005) Acta Biomed 76:44-48).

1. Diabetes Mellitus

Diabetes mellitus (or diabetes) is characterized by an impaired glucose metabolism. Blood glucose is derived from carbohydrates absorbed in the gut and produced in the liver. Increasing blood glucose levels stimulate insulin release. The postprandial glucose influx can be 20 to 30 times higher than the hepatic production of glucose observed between meals. Early phase insulin release, lasting 10 minutes or thereabouts, suppresses hepatic glucose production and precedes a longer (late) phase of release, which lasts two hours or more and covers mealtime carbohydrate influx. Between meals, a low continuous insulin level, basal insulin, covers ongoing metabolic requirements, in particular to regulate hepatic glucose output as well as glucose utilization by adipose tissue, muscle tissue and other target sites. Patients with diabetes present with elevated blood glucose levels (hyperglycemia). Diabetes can be classified into two major groups: type 1 diabetes and type 2 diabetes. Type 1 diabetes, or insulin dependent diabetes mellitus (IDDM), is characterized by a loss of the insulin-producing β-cell of the islets of Langerhans in the pancreas, leading to a deficiency of insulin. The primary cause of the β-cell deficiency is T-cell mediated autoimmunity. Type 2 diabetes, or non-insulin dependent diabetes mellitus (NIDDM), occurs in patients with an impaired β-cell function. These patients have insulin resistance or reduced insulin sensitivity, combined with reduced insulin secretion. Type 2 diabetes may eventually develop into type 1 diabetes. Also included in diabetes is gestational diabetes. Patients with diabetes can be administered insulin to both maintain basal insulin levels and to prevent glycemic excursions, such as following a meal.

a. Type 1 Diabetes

Type 1 diabetes is a T-cell dependent autoimmune disease characterized by infiltration of the islets of Langerhans, the endocrine unit of the pancreas, and destruction of β-cells, leading to a deficiency in insulin production and hyperglycemia. Type 1 diabetes is most commonly diagnosed in children and young adults but can be diagnosed at any age. Patients with type 1 diabetes can present with, in addition to low insulin levels and high blood glucose levels, polyuria, polydipsia, polyphagia, blurred vision and fatigue. Patients can be diagnosed by presenting with fasting plasma glucose levels at or above 126 mg/dL (7.0 mmol/l), plasma glucose levels at or above 200 mg/dL (11.1 mmol/l) two hours after a 75 g oral glucose load, such as in a glucose tolerance test, and/or random plasma glucose levels at or above 200 mg/dL (11.1 mmol/l).

The primary treatment for patients with type 1 diabetes is administration of insulin as replacement therapy, which is typically performed in conjunction with blood glucose monitoring. Without sufficient replacement insulin, diabetic ketoacidosis can develop, which can result in coma or death. Patients can be administered subcutaneous injections of fast-acting insulin using, for example, a syringe or insulin pen, or an insulin pump to maintain appropriate blood glucose levels throughout the day and also to control post-prandial glucose levels. In some instances, an insulin pump, including in the context of a closed loop system, can be used to deliver insulin intraperitoneally. Thus, patients with type 1 diabetes can be administered the co-formulations of a fast acting insulin and hyaluronan degrading enzyme described herein subcutaneously or intraperitoneally via syringe, insulin pen, or insulin pump, or any other means useful for delivering insulin, to more rapidly control blood glucose and insulin levels.

b. Type 2 Diabetes

Type 2 diabetes is associated with insulin resistance and, in some populations, also by insulinopenia (loss of β-cell function). In type 2 diabetes, phase 1 release of insulin is absent, and phase 2 release is delayed and inadequate. The sharp spike of insulin release occurring in healthy subjects during and following a meal is delayed, prolonged, and insufficient in amount in patients with type 2 diabetes, resulting in hyperglycemia. Patients with type 2 diabetes can be administered insulin to control blood glucose levels (Mayfield et al. (2004) Am Earn Physican 70:489-500). This can be done in combination with other treatments and treatment regimes, including diet, exercise and other anti-diabetic therapies (e.g. sulphonylureas, biguanides, meglitinides, thiazolidinediones and alpha-glucosidase inhibitors). Thus, patients with type 2 diabetes can be administered the co-formulations of a fast acting insulin and hyaluronan degrading enzyme described herein subcutaneously or intraperitoneally via syringe, insulin pen, or insulin pump, or any other means useful for delivering insulin, to more rapidly control blood glucose and insulin levels.

c. Gestational Diabetes

Pregnant women who have never had diabetes before but who have high blood glucose levels during pregnancy are diagnosed with gestational diabetes. This type of diabetes affects approximately 1-14% of all pregnant women, depending upon the population studied (Carr et al., (1998) Clinical Diabetes 16). While the underlying cause remains unknown, it appears likely that hormones produced during pregnancy reduce the pregnant woman's sensitivity to insulin. The mechanism of insulin resistance is likely a postreceptor defect, since normal insulin binding by insulin-sensitive cells has been demonstrated. The pancreas releases 1.5-2.5 times more insulin in order to respond to the resultant increase in insulin resistance. Patients with normal pancreatic function are able to meet these demands. Patients with borderline pancreatic function have difficulty increasing insulin secretion and consequently produce inadequate levels of insulin. Gestational diabetes thus results when there is delayed or insufficient insulin secretion in the presence of increasing peripheral insulin resistance.

Patients with gestational diabetes can be administered insulin to control blood glucose level. Thus, patients with gestational diabetes can be administered the co-formulations of a fast acting insulin and hyaluronan degrading enzyme described herein subcutaneously via syringe, insulin pen, insulin pump or artificial pancreas, or any other means, to more rapidly control blood glucose and insulin levels.

2. Insulin Therapy for Critically Ill Patients

Hyperglycemia and insulin resistance occurs frequently in medically and/or surgically critically ill patients and has been associated with increased morbidity and mortality in both diabetic and non-diabetic patients and in patients with traumatic injury, stroke, anoxic brain injury, acute myocardial infarction, post-cardiac surgery, and other causes of critical illness (McCowen et al. (2001) Crit. Clin. Care 17:107-124). Critically ill patients with hyperglycemia have been treated with insulin to control blood glucose levels. Such treatment can reduce morbidity and mortality amongst this group (Van den Berghe et al. (2006) N. Eng. J Med 354:449-461). Insulin is typically administered intravenously to the patient, such as by injection with a syringe by a medical practitioner or by infusion using an insulin pump. In some examples, algorithms and software are used to calculate the dose. Thus, critically ill patients with hyperglycemia can be administered a co-formulation of a fast acting insulin and hyaluronan degrading enzyme described herein to control blood glucose levels, thereby alleviating the hyperglycemia and reducing morbidity and mortality.

### J. COMBINATION THERAPIES

The methods described herein can further include a step of administering, prior to, intermittently with, or subsequent to, other therapeutic agents including but not limited to, other biologics and small molecule compounds. For any disease or condition, including all those exemplified above, for which a fast-acting insulin is indicated or has been used and for which other agents and treatments are available, they can be further used in the methods herein. Depending on the disease or condition to be treated, exemplary other therapeutic agents include, but are not limited to, other anti-diabetic drugs, including, but not limited to, sulfonylureas, biguanides, meglitinides, thiazolidinediones, alpha-glucosidase inhibitors, peptide analogs, including glucagon-like peptide (GLP) analogs and, gastric inhibitory peptide (GIP) analogs and DPP-4 inhibitors. In another example, the methods can further include administering in combination with, prior to, intermittently with, or subsequent to, with one or more other insulins, including fast-acting insulin, and basal-acting insulins.

### K. EXAMPLES

The following examples are included for illustrative purposes only and are not intended to limit the scope of the invention.

### Example 1

### Insulin and Insulin-PH20 Formulations

A. Insulin Aspart

The insulin aspart used in these studies was the commercial product Insulin Aspart: Novo Nordisk, NovoRapid® (insulin Aspart, which is designated NovoLog® in the United States; Lot XS60195). This product contains 100 U/mL insulin aspart, 0.1096 mg/mL zinc, 1.25 mg/mL (7 mM) disodium hydrogen phosphate dihydrate, 0.58 mg/mL (10 mM) NaCl, 16 mg/mL (170 mM) glycerin, 1.5 mg/mL (0.15%) phenol and 1.72 mg/mL (0.172%) m-cresol.

B. Insulin Aspart-PH20 Formulation

The drug product Aspart-PH20 is a sterile, multiple-dose preserved formulation of the active pharmaceutical ingredient recombinant insulin aspart with recombinant human hyaluronidase (rHuPH20, see Examples 5-7) in a neutral pH, buffered isotonic aqueous solution. Each mL of aqueous solution contains insulin aspart (recombinant insulin aspart) 3.50 mg; rHuPH20 (recombinant human hyaluronidase) 5.0 ug; tromethamine (Tris base) 3.63 mg; sodium chloride 2.92 mg; methionine 14.9 mg; poloxamer 188 (Pluronic F68) 0.10 mg; metacresol 0.78 mg; phenol 1.34 mg; and sodium hydroxide and/or hydrochloric acid for pH adjustment to pH 7.4. In some formulations, each mL of aqueous solution contains insulin aspart (recombinant insulin aspart) 3.50 mg; rHuPH20 (recombinant human hyaluronidase) 5.0 ug; tromethamine (Tris base) 3.63 mg; sodium chloride 2.92 mg; methionine 14.9 mg; poloxamer 188 (Pluronic F68) 0.10 mg; metacresol 0.75 mg; phenol 1.25 mg; and sodium hydroxide and/or hydrochloric acid for pH adjustment to pH 7.4.

### Example 2

### Pharmacokinetics (PK) and Glucodynamics of Insulin Aspart and PH20 Formulation by Continuous Subcutaneous Insulin Infusion (CSII)

The insulin aspart formulation (Aspart-PH20) with human hyaluronidase (rHuPH20) described in Example 1 was compared to the commercial insulin aspart formulation (NovoLog®) for three days of diabetes treatment when delivered by continuous subcutaneous infusion in an inpatient setting. Sixteen subjects with type 1 diabetes who were already using continuous subcutaneous insulin infusion (CSII) received each study drug by CSII in random order on either of two visits. The subjects were confined to an inpatient setting for three days of study.

A. Study Protocol

On the afternoon of the first day (day 1) using a Medtronic Paradigm pump system, the subjects had a new infusion site placed and the reservoirs were filled with either aspart-PH20 or Novolog®. The study design allowed comparison of infusion set performance over an observation period of approximately 72 hours.

Twelve to fourteen (12-14) hours after insertion of the new insulin infusion catheter set, a euglycemic glucose clamp experiment was conducted (1^{st} clamp; ½ days after infusion placement). The euglycemic glucose clamps were conducted with a Biostator to provide continuous glucose measurements and adjustment of variable rate intravenous infusion of 20% glucose in water to maintain constant blood glucose levels (Heinemann L, Anderson J H, Jr. Measurement of insulin absorption and insulin action. Diabetes Technol Ther 2004; 6:698-718); a basal intravenous insulin infusion was not employed in this study. Blood glucose was clamped at 90% of the fasting level to suppress endogenous insulin release during the study. A 0.15 U/kg bolus was administered through the insulin pump; and the usual individual basal rate was continued during clamps and PK results are thus baseline-subtracted.

During the euglycemic glucose clamp experiment, the subjects were followed for six (6) hours during which blood was drawn and free insulin levels and glucose infusion rates required to maintain euglycemia were determined. A validated conventional competitive radioimmunoassay (RIA) method was used to determine the insulin aspart concentrations in human serum samples. The tracer and primary antibody used in the RIA were [¹²⁵I]-insulin tracer (Millipore, Catalog #9011) and a guinea pig anti-insulin (Millipore, Catalog #1013-K) antiserum (which cross reacts 100% with human insulin, rat insulin, dog insulin, and insulin lispro). IRI concentrations in the test samples were estimated by interpolation from a standard curve of insulin aspart that ranged in concentration from 10 to 5,000 μM.

Approximately 60 hours after infusion set placement on day 4, and approximately 48 hours after the 1^{st} clamp, the euglycemic glucose clamp experiment was repeated (2^{nd} clamp; 2½ days after infusion placement). The subjects were followed for six (6) hours during which blood was drawn and free insulin levels and glucose infusion rates required to maintain euglycemia were determined as described above.

B. Results

1. Pharmacokinetics of Insulin

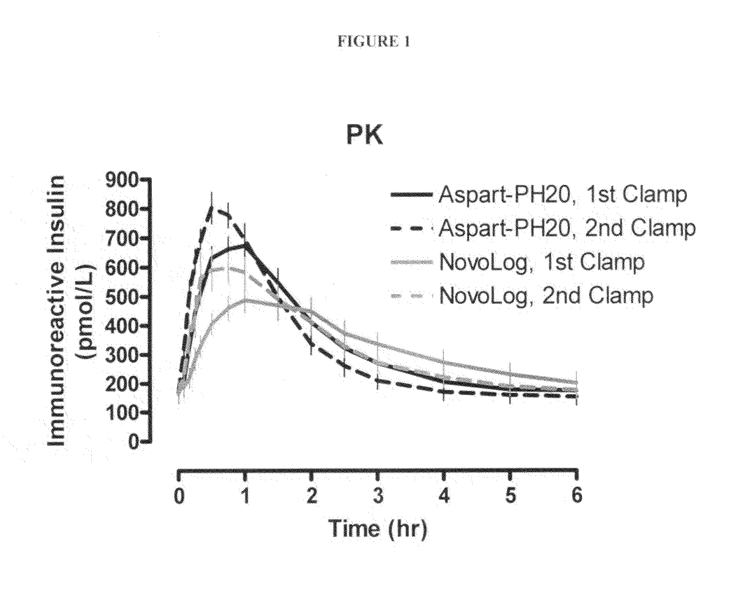

The results for the 1^{st} and 2^{nd} clamp study are depicted as serum immunoreactive insulin (IRI in pmol/L) concentration-versus-time in Table 6. Table 7 depicts the serum immunoreactive insulin results (mean+/−SD). The results also are depicted in FIG. 1.

TABLE 6
Serum Immunoreactive Insulin

| | Aspart-PH20, | | Aspart-PH20, | | NovoLog, 1st | | NovoLog, 2nd | |
|---|---|---|---|---|---|---|---|---|
| Time | 1st Clamp | | 2nd Clamp | | Clamp | | Clamp | |
| (hr) | Mean | SEM | Mean | SEM | Mean | SEM | Mean | SEM |
| 0 | 168 | 35 | 171 | 37 | 176 | 34 | 167 | 32 |
| 0.083 | 213 | 33 | 314 | 49 | 191 | 39 | 229 | 39 |
| 0.167 | 329 | 35 | 508 | 50 | 219 | 36 | 360 | 63 |
| 0.25 | 446 | 37 | 621 | 43 | 285 | 39 | 432 | 73 |
| 0.333 | 512 | 35 | 689 | 43 | 329 | 37 | 565 | 94 |
| 0.5 | 632 | 41 | 803 | 54 | 406 | 38 | 591 | 71 |
| 0.75 | 663 | 42 | 778 | 43 | 461 | 42 | 599 | 52 |
| 1 | 675 | 45 | 695 | 55 | 488 | 42 | 583 | 52 |
| 1.5 | 548 | 48 | 498 | 41 | 471 | 53 | 492 | 51 |
| 2 | 412 | 43 | 338 | 37 | 450 | 48 | 413 | 45 |
| 2.5 | 324 | 42 | 263 | 39 | 374 | 43 | 330 | 39 |
| 3 | 272 | 43 | 212 | 31 | 337 | 40 | 273 | 37 |
| 4 | 206 | 34 | 171 | 29 | 273 | 44 | 223 | 36 |
| 5 | 180 | 33 | 160 | 30 | 232 | 40 | 191 | 35 |
| 6 | 176 | 38 | 155 | 31 | 202 | 39 | 178 | 32 |

TABLE 7
Parameters

| | Aspart CSII Day ½ | | Aspart CSII Day 2½ | |
|---|---|---|---|---|
| | Alone | +rHuPH20 | Alone | +rHuPH20 |
| Early t_{50%} (min) | 26 ± 14 | 17 ± 9 | 17 ± 6 | 11 ± 4 |
| Tₘₐₓ (minutes) | 68 ± 30 | 60 ± 23 | 63 ± 33 | 33 ± 11 |
| Time to 50% AUC (min) | 118 ± 21 | 84 ± 19 | 92 ± 27 | 62 ± 15 |
| Late t_{50%} (min) | 182 ± 40 | 124 ± 38 | 137 ± 48 | 77 ± 28 |
| MRT (min) | 130 ± 19 | 96 ± 21 | 104 ± 29 | 73 ± 23 |
| Cₘₐₓ (pmol/L)^{b} | 344 ± 107 | 554 ± 166 | 549 ± 371 | 700 ± 163 |
| Total AUC | 56 ± 14 | 64 ± 16 | 63 ± 23 | 63 ± 23 |
| (min*nmol/L)^{b} | | | | |
| % AUC 0-60 min | 21 ± 6 | 35 ± 9 | 33 ± 15 | 51 ± 14 |
| % AUC >2 hr | 48 ± 10 | 29 ± 12 | 34 ± 14 | 17 ± 11 |

In the presence of rHuPH20, aspart absorption is accelerated compared to aspart alone after both ½ day CSII (1^{st} clamp) and 2½ days CSII (2^{nd} clamp) (see FIG. 1). For example, ½ day CSII results show insulin exposure in the first hour for the insulin aspart-PH20 formulation was 35% of total AUC and for aspart alone was 21% of total AUC, while exposure beyond 2 hours was 29 and 48%, respectively, of total AUC. 2½ day CSII results show insulin exposure in the first hour for the insulin aspart-PH20 formulation was 51% of total AUC and for aspart alone was 33% of total AUC, while exposure beyond 2 hours was 17 and 34%, respectively, of total AUC. This is consistent with previous studies that show that rHuPH20 accelerates insulin exposure.

For commercial aspart (Novolog®), insulin absorption was accelerated after 2½ days relative to ½ day CSH, with insulin exposure in the first hour increasing from 21 to 33% of total AUC, and exposure beyond 2 hours decreasing from 48 to 34%. For insulin aspart-PH20 formulation, insulin exposure also increased after 2½ days CSII compared to ½ day, with insulin exposure in the first hour increasing from 35 to 51% and exposure beyond 2 hours decreasing from 29 to 17% of total exposure. Absolute insulin exposure in the first hour also increased for insulin aspart from 11.4 nM*Min on day ½ to 21.4 nM*Min on day 2½. This corresponds to a 67% increase in the geometric mean ratio. In addition to the increase in exposure in the first hour on day 2½, the inter-patient variability in exposure was also increased, as the coefficient of variation (CV) increased from 33% to 63%. For the insulin aspart-rHuPH20 formulation, insulin exposure in the first hour increased less, from 22.4 nM*Min on day ½ to 30.0 nM*Min on day 2½. This corresponds to a 39% increase in the geometric mean ratio. The insulin aspart-rHuPH20 formulation also exhibited no increase in inter-patient variability, with the CV actually decreasing slightly from 35% to 28%.

Total insulin exposure (from 0 to 6 hours) was generally the same (no statistically significant difference) for either insulin aspart alone or formulated with rHuPH20 when comparing ½ day to 2½ days of infusion set wear.

2. Glucodynamics

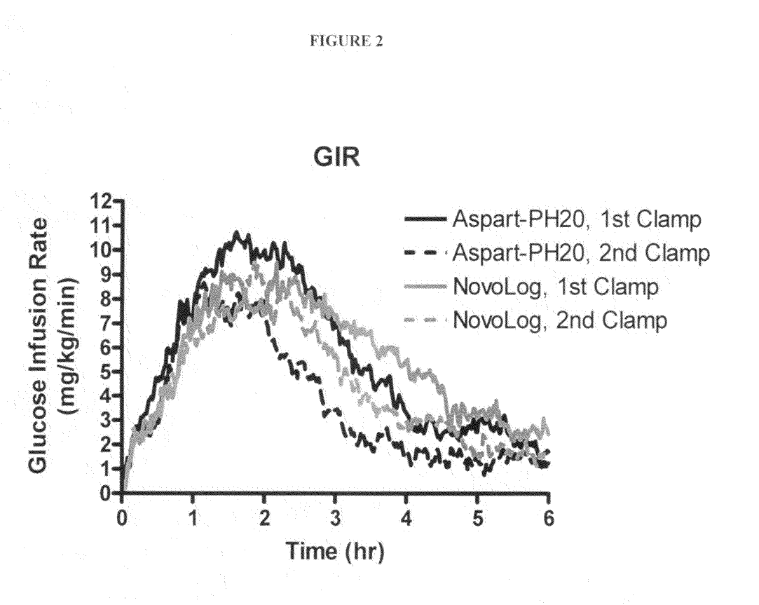

Glucodynamics was measured by determining the infusion rate of glucose necessary to maintain euglycemia following the administration of bolus insulin. The glucodynamic results for each of the treatment groups are summarized in Table 8. The GIR infusion rates are also depicted in FIG. 2. The results are consistent with the acceleration in pharmacokinetics described above.

TABLE 8

| | Aspart CSII Day ½ | | Aspart CSII Day 2½ | |
|---|---|---|---|---|
| | Alone | +rHuPH20 | Alone | +rHuPH20 |
| Early t_{GIRmax50%} | 47 ± 18 | 35 ± 18 | 35 ± 29 | 40 ± 19 |
| (min) | | | | |
| t_{GIRmax} (min) | 115 ± 59 | 102 ± 38 | 91 ± 21 | 84 ± 26 |
| Late t_{GIRmax50%} | 162 ± 57 | 158 ± 53 | 132 ± 47 | 114 ± 29 |
| (min) | | | | |
| Duration of Action | 164 ± 15 | 147 ± 16 | 147 ± 24 | 133 ± 16 |
| (min) | | | | |
| GIRₘₐₓ (mg/kg/min) | 13.4 ± 4.2 | 12.5 ± 3.2 | 11.9 ± 3.6 | 11.5 ± 3.7 |
| Gₜₒₜ (g/kg) | 2.0 ± 0.6 | 2.0 ± 0.8 | 1.6 ± 0.5 | 1.3 ± 0.5 |
| G 0-1 hr | 0.2 ± 0.1 | 0.3 ± 0.1 | 0.2 ± 0.1 | 0.2 ± 0.1 |
| G 0-2 hr | 0.7 ± 0.2 | 0.8 ± 0.3 | 0.7 ± 0.2 | 0.7 ± 0.3 |
| G 0-3 hr | 1.2 ± 0.4 | 1.4 ± 0.5 | 1.1 ± 0.4 | 1.0 ± 0.4 |
| G 0-4 hr | 1.6 ± 0.5 | 1.7 ± 0.6 | 1.3 ± 0.5 | 1.2 ± 0.4 |
| % G 0-½ hr | 2.9 ± 2.1 | 3.6 ± 2.5 | 4.0 ± 2.3 | 4.4 ± 3.0 |
| % G 0-1 hr | 11 ± 4 | 13 ± 5 | 14 ± 6 | 17 ± 8 |
| % G 0-2 hr | 36 ± 7 | 43 ± 9 | 43 ± 13 | 53 ± 7 |
| % G 0-3 hr | 60 ± 8 | 71 ± 9 | 70 ± 12 | 77 ± 7 |
| % G >4 hr | 21 ± 7 | 14 ± 6 | 16 ± 8 | 12 ± 6 |

GIRₘₐₓ: Peak rate of glucose infusion; G (0-1, 0-2, 0-3, 0-4): total glucose infused (g/kg) in time interval

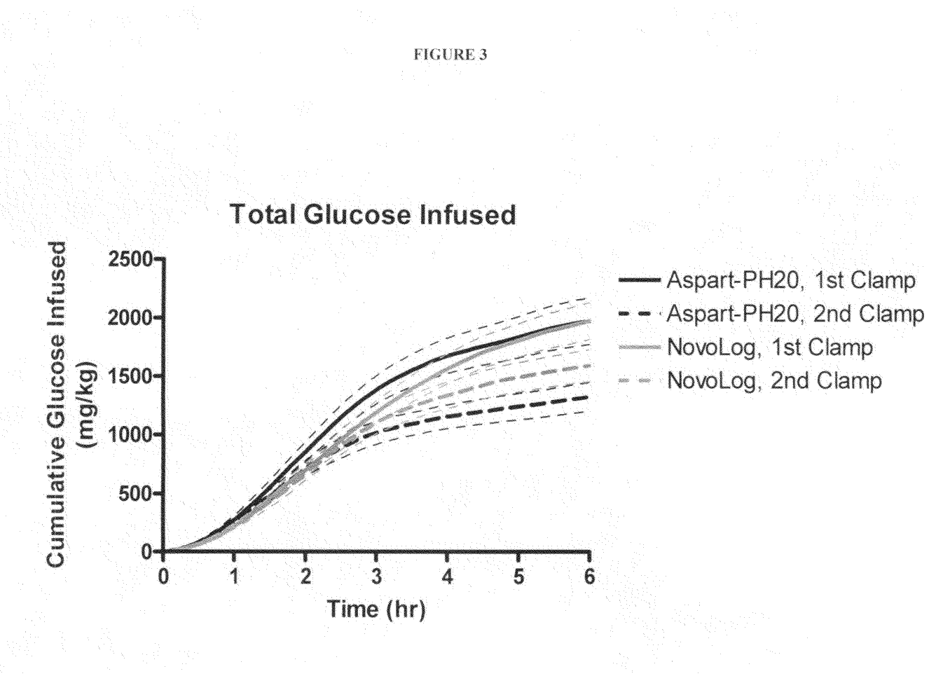

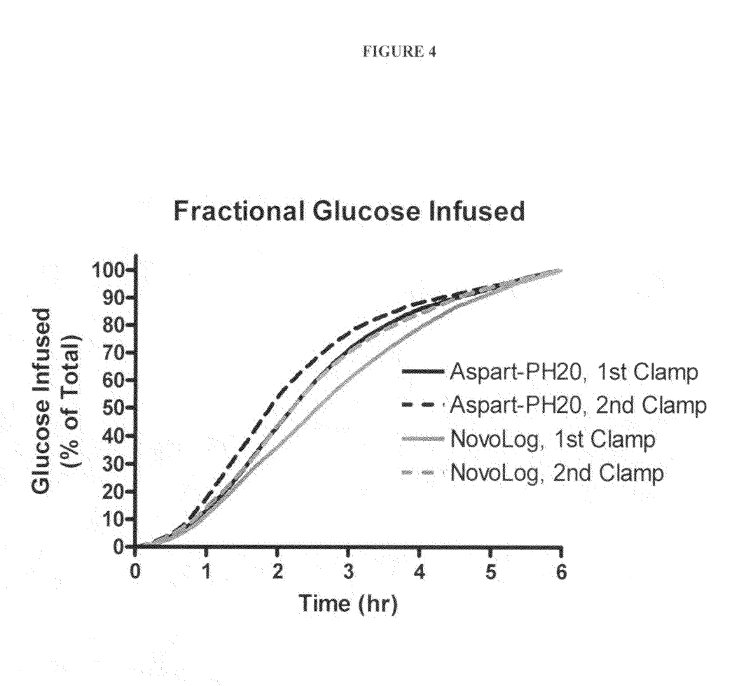

In addition to the faster onset and shorter duration of action seen over the course of infusion set life (1^{st} clamp compared to 2^{nd} clamp), the results also show that total insulin action (Gₜₒₜ; cumulative glucose infused over the course of the experiment) as assayed by the euglycemic clamp method declined over the life of the infusion set. For example, both commercial aspart alone (Novolog®) and insulin aspart-rHuPH20 formulation exhibited the same total insulin action at the time of the 1^{st} clamp, 2.0 g/kg. Two days later at the 2^{nd} clamp, however, the total insulin action was reduced for both study drugs, although to a greater degree for the insulin aspart-rHuPH20 formulation (see FIG. 3). Both treatments (commercial insulin aspart alone or insulin aspart-rHuPH20 formulation) accelerated from the 1^{st} clamp to the 2^{nd} clamp, and the addition of rHuPH20 to insulin aspart resulted in a faster time-action profile as compared to commercial insulin aspart alone at both time points (see FIG. 4).

3. Blood Glucose Response to Meal

The blood glucose response to the meal is described in Table 9.

TABLE 9
Postprandial Glucose Response Parameters (mean)

| PPG Parameter (mg/dL) | With PH20 | Aspart Alone | P-Value* |
|---|---|---|---|
| 1 hr PPG | 125 | 145 | 0.006 |
| 1 hr Excursion | 2 | 25 | 0.077 |
| 90 min PPG | 118 | 146 | 0.055 |
| 90 min Excursion | −5 | 16 | 0.007 |
| 2 hr PPG | 121 | 146 | 0.098 |
| 2 hr Excursion | −2 | 16 | 0.020 |

With aspart-rHuPH20 the meal excursions were consistently well controlled and postprandial hyperglycemia was better than without.

4. Adverse Events Adverse events were assessed during the course or infusion treatment. Table 10 sets forth observed adverse events. The results show that no moderate or severe adverse events were associated with rHuPH20 exposure.

TABLE 10
Adverse Events

| | # (%) of | # (%) of |
|---|---|---|
| | patients | patients |
| | with PH20 | aspart alone |
| | (N = 18) | (N = 20) |
| Any Adverse Event | 13 (72%) | 16 (80%) |
| moderate | 0 | 2 (10%) |
| severe | 0 | 0 |
| All Adverse Events | 23 | 27 |
| Procedural at IV infusion or biopsy sites | 10 | 6 |
| Injection Site | 6 | 5 |
| Headache | 3 | 7 |
| GI | 1 | 3 |
| Musculoskeletal Pain | 0 | 1 |
| Anaemia | 1 | 0 |
| Miscellaneous other events | 2 | 5 |

5. Summary

The results show that rHuPH20 when co-administered with insulin reduces, but does not eliminate, the acceleration of insulin absorption over time after 2½ days relative to ½ day CSII. This is correlated to a reduction in the day-to-day variability in insulin exposure and action as a function of infusion set life. With rHuPH20 present, the data show greater consistency in the time-exposure and total insulin action-normalized time-action profiles.

### Example 3

### Administration of Insulin Aspart with and without PH20 Pretreatment by Continuous Subcutaneous Insulin Infusion (CSII)

The commercial insulin aspart formulation (NovoLog®) was delivered for three days of diabetes treatment by continuous subcutaneous infusion in an inpatient setting. Initially four subjects with type 1 diabetes who were already using continuous subcutaneous insulin infusion (CSII) received NovoLog® by CSII either with or without pretreatment with 150 units (U) of rHuPH20 (prepared as described in Examples 5-7) in random order on either of two visits. The study was continued to include 15 subjects who completed the study protocol, and was further continued to include 17 subjects who completed the study protocol. The subjects were confined to an inpatient setting for three days of study.

A. Study Protocol

On the morning of the first day, the subjects had a new infusion site cannula placed (Medtronic Quick-set) and received either a sham injection or an injection of 1 mL of rHuPH20 (150 U/mL recombinant human hyaluronidase formulated in phosphate buffered saline with 1 mg/mL human serum albumin) through the infusion set and cannula. Immediately after (e.g. within a few minutes) of administration of the rHuPH20, the reservoir was filled with NovoLog® and the patients received insulin by CSII (Medtronic Paradigm pump system) over an observation period of approximately 3 days.

Approximately 2 hours after insertion of the new insulin infusion catheter set, a euglycemic glucose clamp experiment was conducted (1′ clamp). The euglycemic glucose clamps were conducted with a Biostator to provide continuous glucose measurements and adjustment of variable rate intravenous infusion of 20% glucose in water to maintain constant blood glucose levels (Heinemann L, Anderson J H, Jr. Measurement of insulin absorption and insulin action. Diabetes Technol Ther 2004; 6:698-718); a basal intravenous insulin infusion was not employed in this study. Blood glucose was clamped at 90% of the fasting level to suppress endogenous insulin release during the study. A 0.15 U/kg bolus was administered through the insulin pump; and the usual individual basal rate was continued during clamps and PK results are thus baseline-subtracted.

During the euglycemic glucose clamp experiment, the subjects were followed for six (6) hours during which blood was drawn and free insulin levels and glucose infusion rates required to maintain euglycemia were determined. A validated conventional competitive radioimmunoassay (RIA) method was used to determine the insulin aspart concentrations in human serum samples. The tracer and primary antibody used in the RIA were [¹²⁵I]-insulin tracer (Millipore, Catalog #9011) and a guinea pig anti-insulin (Millipore, Catalog #1013-K) antiserum (which cross reacts 100% with human insulin, rat insulin, dog insulin, and insulin lispro). IRI concentrations in the test samples were estimated by interpolation from a standard curve of insulin aspart that ranged in concentration from 10 to 5,000 pM.

Approximately 26 hours after infusion set placement, and approximately 24 hours after the 1^{st} clamp, the euglycemic glucose clamp experiment was repeated (2^{nd} clamp). Approximately 74 hours after infusion set placement, and approximately 48 hours after the 2nd clamp, the euglycemic glucose clamp experiment was again repeated (3^{rd} clamp). In each experiment, the subjects were followed for six (6) hours during which blood was drawn and free insulin levels and glucose infusion rates required to maintain euglycemia were determined as described above.

Patients also received standardized solid evening meals (45-50% CHO, 18-22% protein, 30-34% fat) on each of four consecutive days (approximately 2 hours after a new infusion set without rHuPH20, and after approximately ½, 1½, and 2½ days of infusion set use with or without rHuPH20). Immediately prior to each meal, patients received a patient and meal specific bolus infusion of NovoLog® via the insulin pump, and blood glucose response to the meal was determined.

B. Results

1. Pharmacokinetics of Insulin

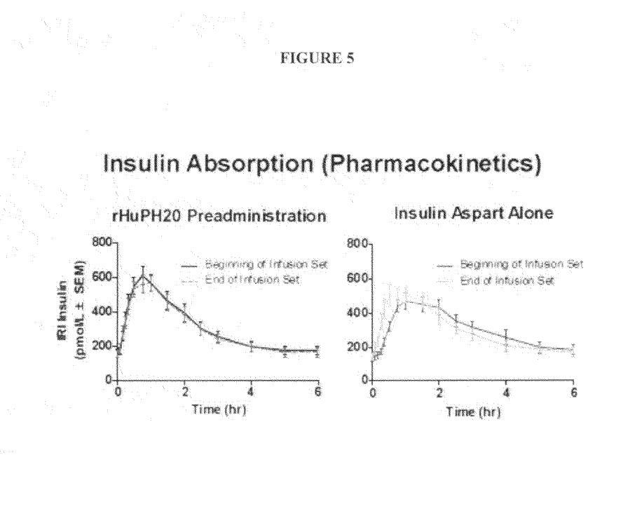

The results from each clamp experiment are presented in Table 11, with results summarized for the 15 completers (Table 11a) and the full 17 completers (Table 11b). The results also are depicted in FIG. 5.

TABLE 11a
Pharmacokinetic Parameters (mean)

| | | Aspart Alone | | | Aspart with rHuPH20 | | |
|---|---|---|---|---|---|---|---|
| | | 1^{st} | 2^{nd} | 3^{rd} | 1^{st} | 2^{nd} | 3^{rd} |
| | | Clamp | Clamp | Clamp | Clamp | Clamp | Clamp |
| Early | | 36.8 | 24.8 | 24.7 | 24.1 | 21.3 | 21.9 |
| t_{50%} (min) | | | | | | | |
| Tₘₐₓ | | 95.0 | 79.0 | 66.3 | 46.2 | 52.3 | 53.0 |
| (minutes) | | | | | | | |
| Late | | 193.5 | 147.2 | 134.3 | 119.8 | 98.5 | 111.6 |
| t_{50%} (min) | | | | | | | |
| MRT (min) | | 143.0 | 120.1 | 116.4 | 103.6 | 91.0 | 99.6 |
| Cₘₐₓ | ^{b} | 369 | 354 | 425 | 474 | 587 | 441 |
| (pmol/L) | | 388.1 | 381.2 | 460.1 | 99.5 | 687.6 | 461.9 |
| Total AUC | ^{b} | 58.9 | 50.7 | 55.1 | 52.4 | 52.8 | 49.9 |
| (min*nmol/ | | 61.1 | 55.6 | 57.7 | 54.3 | 56.1 | 52.4 |
| L) | | | | | | | |

| | | | | | | |
|---|---|---|---|---|---|---|
| % AUC 0-60 | 15.0 | 21.9 | 26.7 | 31.0 | 37.2 | 31.9 |
| min | | | | | | |
| % AUC >2 hr | 53.5 | 42.6 | 39.3 | 32.4 | 25.5 | 30.9 |

^{b} depicted as geometric mean

TABLE 11b
Pharmacokinetic Parameters (mean)

| | Aspart Alone | | | Aspart with rHuPH20 | | |
|---|---|---|---|---|---|---|
| | 1^{st} | 2^{nd} | 3^{rd} | 1^{st} | 2^{nd} | 3^{rd} |
| | Clamp | Clamp | Clamp | Clamp | Clamp | Clamp |
| Early | 35.6 | 23.9 | 24.4 | 23.4 | 21.0 | 21.4 |
| t_{50%} (min) | | | | | | |
| Tₘₐₓ | 89.7 | 74.4 | 63.7 | 44.7 | 50.6 | 51.8 |
| (minutes) | | | | | | |
| Late | 189.9 | 147.9 | 132.3 | 114.8 | 101.0 | 110.9 |
| t_{50%} (min) | | | | | | |
| MRT (min) | 142.3 | 120.6 | 114.7 | 101.7 | 92.6 | 98.6 |
| Cₘₐₓ | 373.6 | 375.1 | 456.2 | 481.7 | 556.3 | 448.9 |
| (pmol/L) | | | | | | |
| Total AUC | 58.8 | 55.4 | 56.6 | 51.7 | 53.7 | 50.7 |
| (min*nmol/ | | | | | | |
| L) | | | | | | |
| % AUC 0-60 | 15.7 | 22.3 | 27.3 | 32.1 | 36.4 | 32.6 |
| min | | | | | | |
| % AUC >2 hr | 53.0 | 42.9 | 38.4 | 31.8 | 26.0 | 30.2 |

With rHuPH20 pretreatment, the insulin was rapidly absorbed throughout the infusion site life. Relative to the 1^{st} clamp without rHuPH20, all clamps with rHuPH20 had characteristic ultrafast profiles, with greater exposure in the 1^{st} hour, greater and earlier peak exposure, and less exposure beyond 2 hours.

Each of the clamps following rHuPH20 pretreatment had similar ultrafast profiles, while each of the clamps without rHuPH20 demonstrated a systematic variation in insulin absorption as the infusion set aged.

2. Glucodynamics

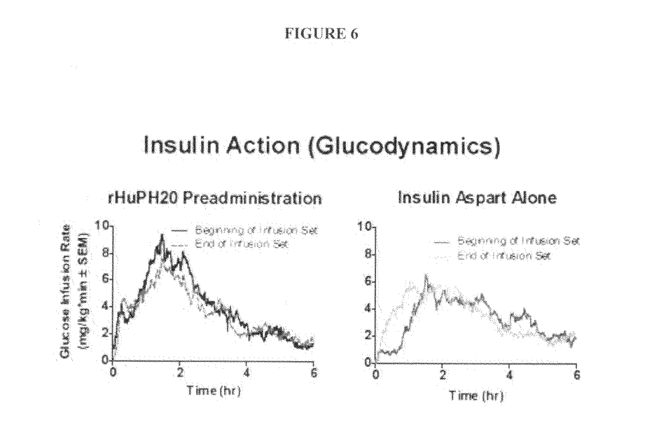

The insulin action profile as a function of time, or glucodynamics, was measured by determining the rate of glucose infusion necessary to maintain euglycemia following the bolus insulin infusion. The results from each clamp experiment are presented in Table 12, with results summarized for the 15 completers (Table 12a) and the full 17 completers (Table 12b). FIG. 6 also depicts the results.

TABLE 12a
Glucodynamic Parameters (mean)

| | Aspart Alone | | | Aspart with rHuPH20 | | |
|---|---|---|---|---|---|---|
| | 1^{st} | 2^{nd} | 3^{rd} | 1^{st} | 2^{nd} | 3^{rd} |
| | Clamp | Clamp | Clamp | Clamp | Clamp | Clamp |
| Early | 60.0 | 33.7 | 29.9 | 34.1 | 32.2 | 31.5 |
| t_{GIRmax50%} | | | | | | |
| (min) | | | | | | |
| t_{GIRmax50%} | 130.1 | 138.5 | 118.7 | 79.5 | 78.9 | 83.5 |
| (min) | | | | | | |
| Late | 144.5 | 157.3 | 138.6 | 97.3 | 103.5 | 113.9 |
| t_{GIRmax50%} | | | | | | |
| (min) | | | | | | |
| GIRmax | 9.6 | 9.7 | 9.6 | 12.3 | 11.6 | 10.5 |
| (mg/kg*min) | | | | | | |
| Duration of | 180.2 | 164.4 | 156.0 | 139.2 | 133.8 | 146.3 |
| Action (min) | | | | | | |
| Gₜₒₜ (g/kg) | 1.16 | 1.30 | 1.21 | 1.45 | 1.36 | 1.30 |
| % G 0-2 hr | 29.3 | 37.4 | 40.6 | 48.9 | 52.0 | 46.7 |

TABLE 12b
Glucodynamic Parameters (mean)

| | Aspart Alone | | | Aspart with rHuPH20 | | |
|---|---|---|---|---|---|---|
| | 1^{st} | 2^{nd} | 3^{rd} | 1^{st} | 2^{nd} | 3^{rd} |
| | Clamp | Clamp | Clamp | Clamp | Clamp | Clamp |
| Early | 58.5 | 32.6 | 27.3 | 31.1 | 31.5 | 28.9 |
| t_{GIRmax50%} | | | | | | |
| (min) | | | | | | |
| t_{GIRmax50%} | 127.7 | 135.2 | 117.8 | 78.5 | 79.5 | 79.5 |
| (min) | | | | | | |
| Late | 144.2 | 153.4 | 136.9 | 98.7 | 102.9 | 110.6 |
| t_{GIRmax50%} | | | | | | |
| (min) | | | | | | |
| GIRmax | 9.6 | 10.1 | 10.0 | 12.6 | 12.0 | 10.8 |
| (mg/kg*min) | | | | | | |
| Duration of | 180.4 | 164.7 | 156.1 | 138.6 | 135.7 | 145.8 |
| Action (min) | | | | | | |
| Gₜₒₜ (g/kg) | 1.20 | 1.37 | 1.29 | 1.51 | 1.41 | 1.37 |
| % G 0-2 hr | 29.5 | 37.4 | 40.7 | 49.5 | 51.3 | 47.1 |

With rHuPH20 pretreatment, the rapid insulin absorption was mirrored with an ultrafast insulin action profile throughout the infusion site life. Relative to the 1^{st} clamp without rHuPH20, all clamps with rHuPH20 had characteristic ultrafast profiles, with greater action in the 1^{st} 1-2 hours, and earlier onset of action (Early t50%), shorter duration of action, and less action beyond 4 hours.

Each of the clamps following rHuPH20 pretreatment had similar ultrafast profiles, while each of the clamps without rHuPH20 demonstrated a systematic variation in insulin action as the infusion set aged.

3. Blood Glucose Response to Meal

The blood glucose response to the meal is described in Table 13, with results summarized for the 15 completers (Table 13a) and the full 17 completers (Table 13b).

TABLE 13a
Postprandial Glucose Response Parameters (mean)

| PPG Parameter (mg/dL) | With PH20 | Aspart Alone | P-Value* |
|---|---|---|---|
| 1 hr PPG | 139.8 | 147.9 | 0.23 |
| 1 hr Excursion | 32.6 | 46.0 | 0.047 |
| 90 min PPG | 124.1 | 141.8 | 0.030 |
| 90 min Excursion | 16.7 | 39.6 | 0.004 |
| 2 hr PPG | 117.1 | 132.8 | 0.073 |
| 2 hr Excursion | 9.9 | 30.6 | 0.017 |

TABLE 13b
Postprandial Glucose Response Parameters (mean)

| PPG Parameter (mg/dL) | With PH20 | Aspart Alone | P-Value* |
|---|---|---|---|
| 1 hr PPG | 135.2 | 140.2 | 0.37 |
| 1 hr Excursion | 29.3 | 39.8 | 0.077 |
| 90 min PPG | 119.0 | 133.4 | 0.055 |
| 90 min Excursion | 12.9 | 32.6 | 0.007 |
| 2 hr PPG | 112.1 | 125.5 | 0.098 |
| 2 hr Excursion | 6.2 | 24.7 | 0.020 |

With rHuPH20 pretreatment the meal excursions were consistently well controlled and postprandial hyperglycemia was better than without.

4. Adverse Events

Adverse events were assessed during the course or infusion treatment. Table 14 sets forth observed adverse events, with results summarized for the 15 completers (Table 14a) and the full 17 completers (Table 14b). The results show that of the adverse events related to CSII infusion sites, two subjects had events associated with rHuPH20 exposure (infusion site pain and infusion site hemorrhage) and one subject had an event associated with insulin aspart alone (infusion site pain).

TABLE 14a
Adverse Events

| | # (%) of | | # (%) of | |
|---|---|---|---|---|
| | patients | | patients | |
| | with PH20 | | aspart alone | |
| | (N = 19) | | (N = 20) | |
| Any Adverse Event | 11 | (57.9%) | 9 | (45.0%) |
| General disorders and administration site | 6 | (31.6%) | 3 | (15.0%) |
| conditions¹ | | | | |
| Nervous system disorders² | 5 | (26.3%) | 3 | (15.0%) |
| Infections and infestations³ | 1 | (5.3%) | 3 | (15.0%) |
| Gastrointestinal disorders⁴ | 2 | (10.5% | 1 | (5.0%) |
| Musculoskeletal and connective tissue | 1 | (5.3%) | 1 | (5.0%) |
| disorders⁵ | | | | |
| skin and subcutaneous tissue disorders⁶ | 1 | (5.3%) | 1 | (5.0%) |

| | | | |
|---|---|---|---|
| Blood and lymphatic system disorders⁷ | 1 | (5.3%) | 0 |
| Metabolism and nutrition disorders⁸ | 1 | (5.3%) | 0 |

¹CSII infusion site pain (n = 2); CSII infusion site hemorrhange (n = 1); peripheral edema (n = 2); the other events were all related to IV infusion sites used for euglycemic clamp procedures
²Primarily headache; dizziness (n = 1), tremor (n = 1)
³IV infusion site infection (n = 1), fungal infection (n = 1), hordeolum (n = 1), IV infusion site cellulitis (n = 1)
⁴Nausea (n = 2), Dyspepsia (n = 1)
⁵Neck pain (n = 1), pain in extremity (n = 1)
⁶Dry skin (n = 1), hyperhidrosis (n = 1)
⁷Anemia (n = 1)
⁸Hypokalemia (n = 1)

TABLE 14b
Adverse Events

| | # (%) of | | # (%) of | |
|---|---|---|---|---|
| | patients | | patients | |
| | with PH20 | | aspart alone | |
| | (N = 22) | | (N = 23) | |
| Any Adverse Event | 12 | (54.5%) | 14 | (60.9%) |
| General disorders and administration site | 6 | (27.3%) | 6 | (26.1%) |
| conditions¹ | | | | |
| Nervous system disorders² | 5 | (22.7%) | 4 | (17.4%) |
| Infections and infestations³ | 1 | (4.5%) | 4 | (17.4%) |
| Gastrointestinal disorders⁴ | 2 | (9.1%) | 1 | (4.3%) |
| Musculoskeletal and connective tissue | 1 | (4.5%) | 2 | (8.7%) |
| disorders⁵ | | | | |
| skin and subcutaneous tissue disorders⁶ | 1 | (4.5%) | 2 | (8.7%) |
| Blood and lymphatic system disorders⁷ | 2 | (9.1%) | 0 | |
| Injury, poisoning and procedural | 1 | (4.5%) | 0 | |
| complications⁸ | | | | |

| | | | |
|---|---|---|---|
| Metabolism and nutrition disorders⁸ | 1 | (4.5%) | 0 |

¹CSII infusion site pain (n = 2); CSII infusion site hemorrhange (n = 1); peripheral edema (n = 2); the other events were all related to IV infusion sites used for euglycemic clamp procedures
²Headache (n = 8), dizziness (n = 1), tremor (n = 1)
³IV infusion site infection (n = 2), fungal infection (n = 1), hordeolum (n = 1), IV infusion site cellulitis (n = 1), vaginal infection (n = 1)
⁴Nausea (n = 2), Dyspepsia (n = 1)
⁵Neck pain (n = 1), pain in extremity (n = 2)
⁶Dry skin (n = 1), ecchymosis (n = 1), hyperhidrosis (n = 1)
⁷Anemia (n = 2)
⁸Burn, 1^{st} degree (n = 1)
⁹Hypokalemia (n = 1)

5. Summary of Results

Consistent with previous reports, insulin absorption and action varied significantly over three days of infusion set use. For example, for the patients treated without rHuPH20, from beginning to end of three days of infusion, results from 15 completers showed early insulin exposure varied from 15 to 27% (p=0.0004), onset of action varied from 60 min to 30 min (p<0.0001), and duration of action varied from 180 to 156 minutes (p=0.0005). Results from 17 completers showed that from beginning to end of three days of infusion, early insulin exposure varied from 16 to 27% (p<0.0001), onset of action varied from 59 min to 27 min (p<0.0001), and duration of action varied from 180 to 156 minutes (p=0.0001).

Pretreatment with rHuPH20 eliminated this variability as there were no significant differences in early insulin exposure, onset or duration of action over three days of continuous infusion. rHuPH20 pretreatment also accelerated insulin absorption. For example, a summary of the results from 15 completers showed the rHuPH20 resulted in 56% more early insulin exposure (P<0.0001), a 9 minute faster onset of action (p=0.037), and a 27 minute shorter duration of action (p<0.0001), and for the 17 completers resulted in a 55% more early insulin exposure (P<0.0001), a 9 minute faster onset of action (p=0.018), and a 27 minute shorter duration of action (p<0.0001). This consistent and ultrafast profile translated into consistently reduced postprandial excursions. For example, a summary of the results from 15 completers showed that the 2 hour postprandial glucose (PPG) was 117 mg/dL and 133 mg/dL without (p=0.073) and for the 17 completers was 112 mg/dL with rHuPH20 and 126 mg/dL without (p=0.098). Also, the reduction in 2 hour glycemic excursion of 21 mg/dL was significant (p=0.017) for the 15 completers. Similarly, the reduction in 2 hour glycemic excursion for the full 17 completers o 19 mg/dL also was significant (p=0.020). Insulin aspart infusion with and without rHuPH20 was similarly well tolerated.

Thus, the results show that preadministration with 150 U of rHuPH20 produced a consistent ultrafast profile for 3½ days of continuous infusion, which provided consistent postprandial control of mixed dinner meals and allowed more patients to consistently achieve target levels of PPG control.

### Example 4

### Administration of Insulin Aspart with and without PH20 Pretreatment by Continuous Subcutaneous Insulin Infusion (CSII)

Patients with type 1 diabetes participated in a randomized, double-blind, 2-way crossover design clinical study comparing the administration of a single hyaluronidase injection at each infusion set change to sham injections in a CSII therapy. The study compared euglycemic clamp endpoints at the beginning and end of 3 days of continuous infusion and glycemic response to a series of four breakfast solid meal challenges. The results are depicted below for the first three subjects that completed the study. In addition, continuous glucose monitoring of the three subjects to compare glucose control in routine outpatient diabetes care also was assessed.

A. Study Protocol

Patients were randomized to receive either sham injection or rHuPH20 hyaluronidase injection (prepared as described in Examples 5-7) for two approximately 16-day treatment periods. In each period, the subjects first presented to the clinical research unit (CRU) to receive a new infusion set as described in Example 3. Briefly, the subjects had a new infusion site cannula placed and received either a sham injection or an injection of 1 mL of rHuPH20 (Hylenex®; 150 USP units of recombinant human hyaluronidase formulated with 8.5 mg sodium chloride, 1.4 mg dibasic sodium phosphate, 1.0 mg albumin human, 0.9 mg edetate disodium, 0.3 mg calcium chloride, pH 7.4). Immediately after (e.g. within a few minutes) of administration of the rHuPH20, the patients received insulin by CSII for infusion over 3 days. Within 4 hours after insertion of the infusion catheter set, a euglycemic clamp experiment was performed as described in Example 3. Subjects were released from the CRU the same day.

Subjects returned 3 days later for a second euglycemic clamp after 3 days of continuous infusion. After the clamp experiment the infusion set was changed and the patients discharged. Over the next 12 days, subjects treated their diabetes normally with unmasked continuous glucose monitoring by sensor augmented CSII covering 4 infusion set cycles each. Subjects returned to the CRU approximately every 3 days to receive a new infusion set and receive a patient specific standardized breakfast meal and insulin bolus. A single administration of 1 mL of 150 Units of rHuPH20 (Hylenex) was administered at the time of each infusion set change. To maintain the double-blind study design, a trained professional not otherwise involved in the study administered either rHuPH20 or a sham injection while the patient looked away. After completion of the phase, patients returned to the CRU within 21 days to repeat these steps with the alternate treatment. During the study patients used their regular insulin pump, infusion set and rapid acting insulin analog, unless incompatible with the hyaluronidase administration procedure (e.g. Omnipod pump, Sure-T infusion set) in which case they were switched to a compatible alternative for the duration of the study.

B. Results

1. Glucodynamics

The insulin action profile as a function of time, or glucodynamics, was measured by determining the rate of glucose infusion necessary to maintain euglycemia following the bolus insulin infusion. The results from each clamp experiment are presented in Table 15.

TABLE 15
Glucodynamic Parameters (mean)

| | Insulin Analog | | Insulin Analog with | |
|---|---|---|---|---|
| | Alone | | rHuPH20 | |
| | 1^{st} Clamp | 2^{nd} Clamp | 1^{st} Clamp | 2^{nd} Clamp |
| Early t_{GIRmax50%} | 66 | 41 | 36 | 29 |
| (min) | | | | |
| t_{GIRmax50%} (min) | 148 | 96 | 87 | 89 |
| Late t_{GIRmax50%} | 168 | 111 | 115 | 132 |
| (min) | | | | |
| GIRmax | 10.9 | 12.1 | 13.9 | 11.2 |
| (mg/kg*min) | | | | |
| Duration of | 160 | 132 | 119 | 113 |
| Action (min) | | | | |
| Gₜₒₜ (g/kg) | 1.14 | 1.14 | 1.30 | 1.12 |
| % G 0-2 hr | 35 | 48 | 58 | 61 |

With rHuPH20 pretreatment, there was an ultrafast insulin action profile throughout the infusion site life. Relative to the 1^{st} clamp without rHuPH20, all clamps with rHuPH20 had characteristic ultrafast profiles, with greater action in the 1^{st} 1-2 hours, and earlier onset of action (Early t50%), shorter duration of action, and less action beyond 4 hours.

Each of the clamps following rHuPH20 pretreatment had similar ultrafast profiles, while each of the clamps without rHuPH20 demonstrated a systematic variation in insulin action as the infusion set aged.

2. Blood Glucose Response to Meal

The blood glucose response to the meal is described in Table 16.

TABLE 16
Postprandial Glucose Response Parameters (mean)

| PPG Parameter (mg/dL) | With PH20 | Insulin analog Alone | P-Value |
|---|---|---|---|
| 1 hr PPG | 143 | 184 | <.0001 |
| 1 hr Excursion | 32 | 74 | <.0001 |
| 90 min PPG | 134 | 175 | <.0001 |
| 90 min Excursion | 23 | 64 | <.0001 |
| 2 hr PPG | 131 | 162 | 0.001 |
| 2 hr Excursion | 20 | 51 | 0.001 |

With rHuPH20 pretreatment the meal excursions were consistently well controlled and postprandial hyperglycemia was better than without rHuPH20 pretreatment.

3. Routine Diabetes Management Endpoints

The first three subjects completing the study represent the initial clinical experience using rHuPH20 preadministration for outpatient control of blood glucose, through approximately 2 weeks of treatment covering 4 infusion set cycles each. All three patients were able to achieve tighter glucose control both lowering their mean CGM glucose and glucose variability, primarily by decreasing hyperglycemia. Hypoglycemic events (determined from symptoms and SMBG records with values ≦70 mg/dL) were mild, and of similar frequency, with 6 episodes during analog alone and 7 episodes after rHuPH20 pretreatment. These results are summarized in Table 17.

TABLE 17
Sensor Glucose Values and Distribution

| Glucose | Sub #1 | Sub #2 | Sub #3 | | | |
|---|---|---|---|---|---|---|
| level | (Analog | (Analog | (Analog | Sub #1 | Sub # 2 | Sub #3 |
| (mg/dL) | alone) | alone) | alone) | (+PH20) | (+PH20) | (+PH20) |
| Mean | 167 | 187 | 232 | 162 | 177 | 202 |
| SD | 74 | 85 | 104 | 63 | 74 | 84 |
| # of Values | N = 2723 | N = 2263 | N = 2547 | N = 2736 | N = 2205 | N = 2524 |
| <70 | 129 (5%) | 101 (4%) | 43 (2%) | 89 (3%) | 53 (2%) | 134 (5%) |
| 70-180 | 1631 (60%) | 1071 (47%) | 941 (37%) | 1810 (66%) | 1314 (60%) | 960 (38%) |
| >180 | 963 (35%) | 1091 (48%) | 1563 (61%) | 837 (31%) | 835 (38%) | 1430 (57%) |
| >240 | 359 (13%) | 541 (24%) | 1123 (44%) | 318 (12%) | 408 (19%) | 804 (32%) |
| Total | 4 | 2 | 0 | 3 | 1 | 0 |
| Hypoglycemic | | | | | | |
| Events | | | | | | |

4. Adverse Events Adverse events were assessed during the course or infusion treatment. Eighteen (18) adverse events were observed in six of eleven evaluable subjects. All were mild and resolved without sequaelae. The most common event was headache (n=4). Potential local site reactions included two (2) instances of pruritis (sham), an abdominal bruise (rHuPH20), pain at the infusion site (rHuPH20) and a stinging sensation during infusion (rHuPH20).

5. Summary of Results

Consistent with previous reports, and Example 3 above, insulin action varied significantly over three days of infusion set use in the absence of rHuPH20 pretreatment. For example, from beginning to end of three days of infusion, onset of action varied from 66 min to 41 min (p=0.01), and duration of action varied from 160 to 132 minutes (p=0.002).

Pretreatment with rHuPH20 eliminated this variability as there were no significant differences in onset or duration of action over three days of continuous infusion. rHuPH20 pretreatment also accelerated insulin action resulting in a 21 minute faster onset of action (p=0.005), and a 30 minute shorter duration of action (p<0.0001). This consistent and ultrafast profile translated into consistently reduced postprandial excursions. For example, the 2 hour postprandial glucose (PPG) was 131 mg/dL with rHuPH20 and 162 mg/dL without (p=0.001).

Thus, the results show that preadministration with 150 U of rHuPH20 produced a consistent ultrafast profile for 3 days of continuous infusion, which provided consistent postprandial control of mixed breakfast meals. Improvements in routine diabetes care parameters also were observed for the initial three subject.

### Example 5

### Generation of a Soluble rHuPH20-Expressing Cell Line

The HZ24 plasmid (set forth in SEQ ID NO: 52) was used to transfect Chinese Hamster Ovary (CHO cells) (see e.g. U.S. Pat. Nos. 7,76,429 and 7,781,607 and U.S. Publication No. 2006-0104968). The HZ24 plasmid vector for expression of soluble rHuPH20 contains a pCI vector backbone (Promega), DNA encoding amino acids 1-482 of human PH20 hyaluronidase (SEQ ID NO:49), an internal ribosomal entry site (IRES) from the ECMV virus (Clontech), and the mouse dihydrofolate reductase (DHFR) gene. The pCI vector backbone also includes DNA encoding the Beta-lactamase resistance gene (AmpR), an f1 origin of replication, a Cytomegalovirus immediate-early enhancer/promoter region (CMV), a chimeric intron, and an SV40 late polyadenylation signal (SV40). The DNA encoding the soluble rHuPH20 construct contains an NheI site and a Kozak consensus sequence prior to the DNA encoding the methionine at amino acid position 1 of the native 35 amino acid signal sequence of human PH20, and a stop codon following the DNA encoding the tyrosine corresponding to amino acid position 482 of the human PH20 hyaluronidase set forth in SEQ ID NO:1, followed by a BamHI restriction site. The construct pCI-PH20-IRES-DHFR-SV40pa (HZ24), therefore, results in a single mRNA species driven by the CMV promoter that encodes amino acids 1-482 of human PH20 (set forth in SEQ ID NO:3) and amino acids 1-186 of mouse dihydrofolate reductase (set forth in SEQ ID NO:53), separated by the internal ribosomal entry site (IRES).

Non-transfected DG44 CHO cells growing in GIBCO Modified CD-CHO media for DHFR(−) cells, supplemented with 4 mM Glutamine and 18 ml/L Plurionic F68/L (Gibco), were seeded at 0.5×10⁶ cells/fill in a shaker flask in preparation for transfection. Cells were grown at 37° C. in 5% CO, in a humidified incubator, shaking at 120 rpm. Exponentially growing non-transfected DG44 CHO cells were tested for viability prior to transfection.

Sixty million viable cells of the non-transfected DG44 CHO cell culture were pelleted and resuspended to a density of 2×10⁷ cells in 0.7 mL of 2× transfection buffer (2×HeBS: 40 mM Hepes, pH 7.0, 274 mM NaCl, 10 mM KCl, 1.4 mM Na₂HPO₄, 12 mM dextrose). To each aliquot of resuspended cells, 0.09 mL (250 μg) of the linear HZ24 plasmid (linearized by overnight digestion with Cla I (New England Biolabs) was added, and the cell/DNA solutions were transferred into 0.4 cm gap BTX (Centronics) electroporation cuvettes at room temperature. A negative control electroporation was performed with no plasmid DNA mixed with the cells. The cell/plasmid mixes were electroporated with a capacitor discharge of 330 V and 960 μF or at 350 V and 960 μF.

The cells were removed from the cuvettes after electroporation and transferred into 5 mL of Modified CD-CHO media for DHFR(−) cells, supplemented with 4 mM Glutamine and 18 ml/L Plurionic F68/L (Gibco), and allowed to grow in a well of a E-well tissue culture plate without selection for 2 days at 37° C. in 5% CO, in a humidified incubator.

Two days post-electroporation, 0.5 mL of tissue culture media was removed from each well and tested for the presence of hyaluronidase activity, using the microturbidity assay described in Example 8.

TABLE 18
Initial Hyaluronidase Activity of HZ24 Transfected
DG44 CHO cells at 40 hours post-transfection

| | Dilution | Activity (Units/ml) |
|---|---|---|
| Transfection 1 330 V | 1 to 10 | 0.25 |
| Transfection 2 350 V | 1 to 10 | 0.52 |
| Negative Control | 1 to 10 | 0.015 |

Cells from Transfection 2 (350V) were collected from the tissue culture well, counted and diluted to 1×10⁴ to 2×10⁴ viable cells per mL. A 0.1 mL aliquot of the cell suspension was transferred to each well of five, 96 well round bottom tissue culture plates. One hundred microliters of CD-CHO media (GIBCO) containing 4 mM GlutaMAX™-1 supplement (GIBCO™, Invitrogen Corporation) and without hypoxanthine and thymidine supplements were added to the wells containing cells (final volume 0.2 mL).

Ten clones were identified from the 5 plates grown without methotrexate.

TABLE 19
Hyaluronidase activity of identified clones

| Plate/Well ID | Relative Hyaluronidase |
|---|---|
| 1C3 | 261 |
| 2C2 | 261 |
| 3D3 | 261 |
| 3E5 | 243 |
| 3C6 | 174 |
| 2G8 | 103 |
| 1B9 | 304 |
| 2D9 | 273 |
| 4D10 | 302 |

Six HZ24 clones were expanded in culture and transferred into shaker flasks as single cell suspensions. Clones 3D3, 3E5, 2G8, 2D9, 1E11, and 4D10 were plated into 96-well round bottom tissue culture plates using a two-dimensional infinite dilution strategy in which cells were diluted 1:2 down the plate, and 1:3 across the plate, starting at 5000 cells in the top left hand well. Diluted clones were grown in a background of 500 non-transfected DG44 CHO cells per well, to provide necessary growth factors for the initial days in culture. Ten plates were made per subclone, with plates containing 50 nM methotrexate and 5 plates without methotrexate.

Clone 3D3 produced 24 visual subclones (13 from the no methotrexate treatment, and 11 from the 50 nM methotrexate treatment. Significant hyaluronidase activity was measured in the supernatants from 8 of the 24 subclones (>50 Units/mL), and these 8 subclones were expanded into T-25 tissue culture flasks. Clones isolated from the methotrexate treatment protocol were expanded in the presence of 50 nM methotrexate. Clone 3D35M was further expanded in 500 nM methotrexate giving rise to clones producing in excess of 1,000 Units/ml in shaker flasks (clone 3D35M; or Gen1 3D35M). A master cell bank (MCB) of the 3D35M cells was then prepared.

### Example 6

### Production Gen2 Cells Containing Soluble Human PH20 (rHuPH20)

The Gen1 3D35M cell line described in Example 5 was adapted to higher methotrexate levels to produce generation 2 (Gen2) clones. 3D35M cells were seeded from established methotrexate-containing cultures into CD CHO medium containing 4 mM GlutaMAX-1™ and 1.0 μM methotrexate. The cells were adapted to a higher methotrexate level by growing and passaging them 9 times over a period of 46 days in a 37° C., 7% CO, humidified incubator. The amplified population of cells was cloned out by limiting dilution in 96-well tissue culture plates containing medium with 2.0 μM methotrexate. After approximately 4 weeks, clones were identified and clone 3E10B was selected for expansion. 3E10B cells were grown in CD CHO medium containing 4 mM GlutaMAX-1™ and 2.0 μM methotrexate for 20 passages. A master cell bank (MCB) of the 3E10B cell line was created and frozen and used for subsequent studies.

Amplification of the cell line continued by culturing 3E10B cells in CD CHO medium containing 4 mM GlutaMAX-1™ and 4.0 μM methotrexate. After the 12^{th} passage, cells were frozen in vials as a research cell bank (RCB). One vial of the RCB was thawed and cultured in medium containing 8.0 μM methotrexate. After 5 days, the methotrexate concentration in the medium was increased to 16.0 μM, then 20.0 μM 18 days later. Cells from the 8^{th} passage in medium containing 20.0 μM methotrexate were cloned out by limiting dilution in 96-well tissue culture plates containing CD CHO medium containing 4 mM GlutaMAX-1™ and 20.0 μM methotrexate. Clones were identified 5-6 weeks later and clone 2B2 was selected for expansion in medium containing 20.0 μM methotrexate. After the 11th passage, 2B2 cells were frozen in vials as a research cell bank (RCB).

The resultant 2B2 cells are dihydrofolate reductase deficient (dhfr−) DG44 CHO cells that express soluble recombinant human PH20 (rHuPH20). The soluble PH20 is present in 2B2 cells at a copy number of approximately 206 copies/cell. Southern blot analysis of Spe I-, Xba I- and BamH I/Hind III-digested genomic 2B2 cell DNA using a rHuPH20-specific probe revealed the following restriction digest profile: one major hybridizing band of ˜7.7 kb and four minor hybridizing bands (˜13.9, ˜6.6, ˜5.7 and ˜4.6 kb) with DNA digested with Spe I; one major hybridizing band of ˜5.0 kb and two minor hybridizing bands (˜13.9 and ˜6.5 kb) with DNA digested with Xba I; and one single hybridizing band of ˜1.4 kb observed using 2B2 DNA digested with BamH I/Hind III. Sequence analysis of the mRNA transcript indicated that the derived cDNA (SEQ ID NO:56) was identical to the reference sequence (SEQ ID NO:49) except for one base pair difference at position 1131, which was observed to be a thymidine (T) instead of the expected cytosine (C). This is a silent mutation, with no effect on the amino acid sequence.

### Example 7

### A. Production of Gen2 Soluble rHuPH20 in 300 L Bioreactor Cell Culture

A vial of HZ24-2B2 was thawed and expanded from shaker flasks through 36 L spinner flasks in CD-CHO media (Invitrogen, Carlsbad, Calif.) supplemented with 20 μM methotrexate and GlutaMAX-1™ (Invitrogen). Briefly, the a vial of cells was thawed in a 37° C. water bath, media was added and the cells were centrifuged. The cells were re-suspended in a 125 mL shake flask with 20 mL of fresh media and placed in a 37° C., 7% CO, incubator. The cells were expanded up to 40 mL in the 125 mL shake flask. When the cell density reached greater than 1.5×10⁶ cells/mL, the culture was expanded into a 125 mL spinner flask in a 100 mL culture volume. The flask was incubated at 37° C., 7% CO₂. When the cell density reached greater than 1.5×10⁶ cells/mL, the culture was expanded into a 250 mL spinner flask in 200 mL culture volume, and the flask was incubated at 37° C., 7% CO, When the cell density reached greater than 1.5×10⁶ cells/mL, the culture was expanded into a 1 L spinner flask in 800 mL culture volume and incubated at 37° C., 7% CO₂. When the cell density reached greater than 1.5×10⁶ cells/mL the culture was expanded into a 6 L spinner flask in 5000 mL culture volume and incubated at 37° C., 7% CO₂. When the cell density reached greater than 1.5×10⁶ cells/mL the culture was expanded into a 36 L spinner flask in 32 L culture volume and incubated at 37° C., 7% CO₂.

A 400 L reactor was sterilized and 230 mL of CD-CHO media was added. Before use, the reactor was checked for contamination. Approximately 30 L cells were transferred from the 36 L spinner flasks to the 400 L bioreactor (Braun) at an inoculation density of 4.0×10⁵ viable cells per ml and a total volume of 260 L. Parameters were temperature set point, 37° C.; Impeller Speed 40-55 RPM; Vessel Pressure: 3 psi; Air Sparge 0.5-1.5 L/Min.; Air Overlay: 3 L/min. The reactor was sampled daily for cell counts, pH verification, media analysis, protein production and retention. Also, during the run nutrient feeds were added. At 120 hrs (day 5), 10.4 L of Feed #1 Medium (4×CD-CHO+33 g/L Glucose+160 mL/L Glutamax-1™+83 mL/L Yeastolate+33 mg/L rHulnsulin) was added. At 168 hours (day 7), 10.8 L of Feed #2 (2×CD-CHO+33 g/L Glucose+80 mL/L Glutamax-1™+167 mL/L Yeastolate+0.92 g/L Sodium Butyrate) was added, and culture temperature was changed to 36.5° C. At 216 hours (day 9), 10.8 L of Feed #3 (1×CD-CHO+50 g/L Glucose+50 mL/L Glutamax-1™+250 mL/L Yeastolate+1.80 g/L Sodium Butyrate) was added, and culture temperature was changed to 36° C. At 264 hours (day 11), 10.8 L of Feed #4 (1× CD-CHO+33 g/L Glucose+33 mL/L Glutamax-1™+250 mL/L Yeastolate+0.92 g/L Sodium Butyrate) was added, and culture temperature was changed to 35.5° C. The addition of the feed media was observed to dramatically enhance the production of soluble rHuPH20 in the final stages of production. The reactor was harvested at 14 or 15 days or when the viability of the cells dropped below 40%. The process resulted in a final productivity of 17,000 Units per ml with a maximal cell density of 12 million cells/mL. At harvest, the culture was sampled for mycoplasma, bioburden, endotoxin and viral in vitro and in vivo, Transmission Electron Microscopy (TEM) and enzyme activity.

The culture was pumped by a peristaltic pump through four Millistak filtration system modules (Millipore) in parallel, each containing a layer of diatomaceous earth graded to 4-8 μm and a layer of diatomaceous earth graded to 1.4-1.1 μm, followed by a cellulose membrane, then through a second single Millistak filtration system (Millipore) containing a layer of diatomaceous earth graded to 0.4-0.11 μm and a layer of diatomaceous earth graded to <0.1 μm, followed by a cellulose membrane, and then through a 0.22 μm final filter into a sterile single use flexible bag with a 350 L capacity. The harvested cell culture fluid was supplemented with 10 mM EDTA and 10 mM Tris to a pH of 7.5. The culture was concentrated 10× with a tangential flow filtration (TFF) apparatus using four. Sartoslice TFF 30 kDa molecular weight cut-off (MWCO) polyether sulfone (PES) filter (Sartorius), followed by a 10× buffer exchange with 10 mM Tris, 20 mM Na₂SO₄, pH 7.5 into a 0.22 μm final filter into a 50 L sterile storage bag.

The concentrated, diafiltered harvest was inactivated for virus. Prior to viral inactivation, a solution of 10% Triton X-100, 3% tri (n-butyl) phosphate (TNBP) was prepared. The concentrated, diafiltered harvest was exposed to 1% Triton X-100, 0.3% TNBP for 1 hour in a 36 L glass reaction vessel immediately prior to purification on the Q column.

B. Purification of Gen2 Soluble rHuPH20

A Q Sepharose (Pharmacia) ion exchange column (9 L resin, H=29 cm, D=20 cm) was prepared. Wash samples were collected for a determination of pH, conductivity and endotoxin (LAL) assay. The column was equilibrated with 5 column volumes of 10 mM Tris, 20 mM Na₂SO₄, pH 7.5. Following viral inactivation, the concentrated, diafiltered harvest was loaded onto the Q column at a flow rate of 100 cm/hr. The column was washed with 5 column volumes of 10 mM Tris, 20 mM Na₂SO₄, pH 7.5 and 10 mM Hepes, 50 mM NaCl, pH 7.0. The protein was eluted with 10 mM Hepes, 400 mM NaCl, pH 7.0 into a 0.22 μm final filter into sterile bag. The eluate sample was tested for bioburden, protein concentration and hyaluronidase activity. A₂₈₀ absorbance reading were taken at the beginning and end of the exchange.

Phenyl-Sepharose (Pharmacia) hydrophobic interaction chromatography was next performed. A Phenyl-Sepharose (PS) column (19-21 L resin, H=29 cm, D=30 cm) was prepared. The wash was collected and sampled for pH, conductivity and endotoxin (LAL assay). The column was equilibrated with 5 column volumes of 5 mM potassium phosphate, 0.5 M ammonium sulfate, 0.1 mM CaCl2, pH 7.0. The protein eluate from the Q sepharose column was supplemented with 2M ammonium sulfate, 1 M potassium phosphate and 1 M CaCl₂ stock solutions to yield final concentrations of 5 mM, 0.5 M and 0.1 mM, respectively. The protein was loaded onto the PS column at a flow rate of 100 cm/hr and the column flow thru collected. The column was washed with 5 mM potassium phosphate, 0.5 M ammonium sulfate and 0.1 mM CaCl2 pH 7.0 at 100 cm/hr and the wash was added to the collected flow thru. Combined with the column wash, the flow through was passed through a 0.22 μm final filter into a sterile bag. The flow through was sampled for bioburden, protein concentration and enzyme activity.

An aminophenyl boronate column (ProMedics) was prepared. The wash was collected and sampled for pH, conductivity and endotoxin (LAL assay). The column was equilibrated with 5 column volumes of 5 mM potassium phosphate, 0.5 M ammonium sulfate. The PS flow through containing purified protein was loaded onto the aminophenyl boronate column at a flow rate of 100 cm/hr. The column was washed with 5 mM potassium phosphate, 0.5 M ammonium sulfate, pH 7.0. The column was washed with 20 mM bicine, 0.5 M ammonium sulfate, pH 9.0. The column was washed with 20 mM bicine, 100 mM sodium chloride, pH 9.0. The protein was eluted with 50 mM Hepes, 100 mM NaCl, pH 6.9 and passed through a sterile filter into a sterile bag. The eluted sample was tested for bioburden, protein concentration and enzyme activity.

The hydroxyapatite (HAP) column (Biorad) was prepared. The wash was collected and test for pH, conductivity and endotoxin (LAL assay). The column was equilibrated with 5 mM potassium phosphate, 100 mM NaCl, 0.1 mM CaCl₂, pH 7.0. The aminophenyl boronate purified protein was supplemented to final concentrations of 5 mM potassium phosphate and 0.1 mM CaCh and loaded onto the HAP column at a flow rate of 100 cm/hr. The column was washed with 5 mM potassium phosphate, pH 7, 100 mM NaCl, 0.1 mM CaCl₂. The column was next washed with 10 mM potassium phosphate, pH 7, 100 mM NaCl, 0.1 mM CaCl₂. The protein was eluted with 70 mM potassium phosphate, pH 7.0 and passed through a 0.22 μm sterile filter into a sterile bag. The eluted sample was tested for bioburden, protein concentration and enzyme activity.

The HAP purified protein was then passed through a viral removal filter. The sterilized Viosart filter (Sartorius) was first prepared by washing with 2 L of 70 mM potassium phosphate, pH 7.0. Before use, the filtered buffer was sampled for pH and conductivity. The HAP purified protein was pumped via a peristaltic pump through the 20 nM viral removal filter. The filtered protein in 70 mM potassium phosphate, pH 7.0 was passed through a 0.22 μm final filter into a sterile bag. The viral filtered sample was tested for protein concentration, enzyme activity, oligosaccharide, monosaccharide and sialic acid profiling. The sample also was tested for process related impurities.

The protein in the filtrate was then concentrated to 10 mg/mL using a 10 kD molecular weight cut off (MWCO) Sartocon Slice tangential flow filtration (TFF) system (Sartorius). The filter was first prepared by washing with 10 mM histidine, 130 mM NaCl, pH 6.0 and the permeate was sampled for pH and conductivity. Following concentration, the concentrated protein was sampled and tested for protein concentration and enzyme activity. A 6× buffer exchange was performed on the concentrated protein into the final buffer: 10 mM histidine, 130 mM NaCl, pH 6.0. Following buffer exchange, the concentrated protein was passed though a 0.22 filter into a 20 L sterile storage bag. The protein was sampled and tested for protein concentration, enzyme activity, free sulfhydryl groups, oligosaccharide profiling and osmolality.

The sterile filtered bulk protein was then asceptically dispensed at 20 mL into 30 mL sterile Teflon vials (Nalgene). The vials were then flash frozen and stored at −20±5° C.

### Example 8

### Determination of Hyaluronidase Activity of rHuPH20

Hyaluronidase activity of rHuPH20 (obtained by expression and secretion in CHO cells of a nucleic acid encoding amino acids 36-482 of SEQ ID NO:1) was determined using a turbidimetric assay. In the first two assay (A and B), the hyaluronidase activity of rHuPH20 was measured by incubating soluble rHuPH20 with sodium hyaluronate (hyaluronic acid) and then precipitating the undigested sodium hyaluronate by addition of acidified serum albumin. In the third assay (C), rHuPH20 hyaluronidase activity was measured based on the formation of an insoluble precipitate when hyaluronic acid (HA) binds with cetylpyridinium chloride (CPC). In all assays containing 600 U/mL rHuPH20 (5 μg/mL), the acceptance criteria was enzymatic activity above 375 U/mL.

A. Microturbidity Assay

In this assay, the hyaluronidase activity of rHuPH20 was measured by incubating soluble rHuPH20 with sodium hyaluronate (hyaluronic acid) for a set period of time (10 minutes) and then precipitating the undigested sodium hyaluronate with the addition of acidified serum albumin. The turbidity of the resulting sample was measured at 640 nm after a 30 minute development period. The decrease in turbidity resulting from enzyme activity on the sodium hyaluronate substrate was a measure of the soluble rHuPH20 hyaluronidase activity. The method was performed using a calibration curve generated with dilutions of a soluble rHuPH20 assay working reference standard, and sample activity measurements were made relative to this calibration curve. Dilutions of the sample were prepared in Enzyme Diluent Solutions. The Enzyme Diluent Solution was prepared by dissolving 33.0±0.05 mg of hydrolyzed gelatin in 25.0 mL of 50 mM PIPES Reaction Buffer (140 mM NaCl, 50 mM PIPES, pH 5.5) and 25.0 mL of Sterile Water for Injection (SWFI; Braun, product number RS000-1) and diluting 0.2 mL of a 25% Human Serum Albumin (US Biologicals) solution into the mixture and vortexing for 30 seconds. This was performed within 2 hours of use and stored on ice until needed. The samples were diluted to an estimated 1-2 U/mL. Generally, the maximum dilution per step did not exceed 1:100 and the initial sample size for the first dilution was not be less than 20 μL. The minimum sample volumes needed to perform the assay were: In-process Samples, FPLC Fractions: 80 μL; Tissue Culture Supernatants: 1 mL; Concentrated Material 80 μL; Purified or Final Step Material: 80 μL. The dilutions were made in triplicate in a Low Protein Binding 96-well plate, and 30 μL of each dilution was transferred to Optilux black/clear bottom plates (BD BioSciences).

Dilutions of known soluble rHuPH20 with a concentration of 2.5 U/mL were prepared in Enzyme Diluent Solution to generate a standard curve and added to the Optilux plate in triplicate. The dilutions included 0 U/mL, 0.25 U/mL, 0.5 U/mL, 1.0 U/mL, 1.5 U/mL, 2.0 U/mL, and 2.5 U/mL. “Reagent blank” wells that contained 60 μL of Enzyme Diluent Solution were included in the plate as a negative control. The plate was then covered and warmed on a heat block for 5 minutes at 37° C. The cover was removed and the plate was shaken for 10 seconds. After shaking, the plate was returned to the plate to the heat block and the MULTIDROP 384 Liquid Handling Device was primed with the warm 0.25 mg/mL sodium hyaluronate solution (prepared by dissolving 100 mg of sodium hyaluronate (LifeCore Biomedical) in 20.0 mL of SWFI. This was mixed by gently rotating and/or rocking at 2-8° C. for 2-4 hours, or until completely dissolved). The reaction plate was transferred to the MULTIDROP 384 and the reaction was initiated by pressing the start key to dispense 30 μL sodium hyaluronate into each well. The plate was then removed from the MULTIDROP 384 and shaken for 10 seconds before being transferred to a heat block with the plate cover replaced. The plate was incubated at 37° C. for 10 minutes.

The MULTIDROP 384 was prepared to stop the reaction by priming the machine with Serum Working Solution and changing the volume setting to 240 μL. (25 mL of Serum Stock Solution [1 volume of Horse Serum (Sigma) was diluted with 9 volumes of 500 mM Acetate Buffer Solution and the pH was adjusted to 3.1 with hydrochloric acid] in 75 mL of 500 mM Acetate Buffer Solution). The plate was removed from the heat block and placed onto the MULTIDROP 384 and 240 μL of serum Working Solutions was dispensed into the wells. The plate was removed and shaken on a plate reader for 10 seconds. After a further 15 minutes, the turbidity of the samples was measured at 640 nm and the hyaluronidase activity (in U/mL) of each sample was determined by fitting to the standard curve.

Specific activity (Units/mg) was calculated by dividing the hyaluronidase activity (U/ml) by the protein concentration (mg/mL).

B. Turbidity Assay for rHuPH20 Enzymatic Activity

Samples were diluted with Enzyme Diluent [66 mg gelatin hydrolysate (Sigma #G0262) dissolved in 50 mL Phosphate Buffer (25 mM phosphate, pH 6.3, 140 mM NaCl) and 50 mL deionized (DI) water] to achieve an expected enzyme concentration of between 0.3 and 1.5 U/mL.

Each of two test tubes labeled Standard 1, 2, 3, 4, 5, or 6, and duplicate test tubes for each sample to be analyzed (labeled accordingly) were placed in a block heater at 37° C. The volumes of Enzyme Diluent shown in the following table were added in duplicate to the Standard test tubes. 0.50 mL HA Substrate Solution [1.0 mL of 5 mg/mL hyaluronic acid (ICN #362421) in DI water, 9 mL DI water, 10 mL Phosphate Buffer] was dispensed into all the Standard and Sample test tubes. Volumes of 1.5 U/mL USP Hyaluronidase Standard (USP #31200) in Enzyme Diluent were dispensed into duplicate Standard test tubes as indicated in the Table 12 below. When all the Standard test tubes had been completed, 0.50 mL of each sample was dispensed into each of the duplicate Sample test tubes. After a 30-minute incubation at 37° C., 4.0 mL of Serum Working Solution {50 mL Serum Stock Solution [1 volume horse serum (donor herd, cell culture tested, hybridoma culture tested, USA origin), 9 volumes 500 mM Acetate Buffer, adjust to pH 3.1, allow to stand at room temperature 18-24 hours, store at 4° C.] plus 150 mL 500 mM Acetate Buffer} was added to the Standard test tubes, which were then removed from the block heater, mixed and placed at room temperature. The Sample test tubes were processed in this manner until all of the Standard and Sample test tubes were processed.

A “blank” solution was prepared by combining 0.5 mL Enzyme Diluent, 0.25 mL DI water, 0.25 mL Phosphate Buffer and 4.0 mL Serum Working Solution. The solution was mixed and an aliquot transferred to a disposable cuvette. This sample was used to zero the spectrophotometer at 640 nm.

After a 30-minute incubation at room temperature an aliquot from each Standard test tube was transferred in turn to a disposable cuvette and the absorbance at 640 nm was measured. This procedure was repeated for the duplicate Sample test tubes.

A linear calibration curve was constructed by plotting the hyaluronidase concentration (U/mL) versus the observed absorbance. Linear regression analysis was used to fit the data (excluding the data for the 0.0 U/mL calibration standard) and to determine the slope, intercept and correlation coefficient (r²). A standard curve regression equation and the observed sample absorbance were used to determine the sample concentrations.

TABLE 20
Dilutions for Enzyme Standards

| | | mL Enzyme | mL 1.5 U/mL USP |
|---|---|---|---|
| Standard | U/mL | Diluent | Hyaluronidase |
| 1 | 0.0 | 0.50 | 0 |
| 2 | 0.3 | 0.40 | 0.10 |
| 3 | 0.6 | 0.30 | 0.20 |
| 4 | 0.9 | 0.20 | 0.30 |
| 5 | 1.2 | 0.10 | 0.40 |
| 6 | 1.5 | 0 | 0.50 |

C. Turbidity Assay for rHuPH20 Enzymatic Activity

The turbidimetric method for the determination of hyaluronidase activity and enzyme concentration was based on the formation of an insoluble precipitate when hyaluronic acid (HA) binds with cetylpyridinium chloride (CPC). The activity was measured by incubating hyaluronidase with hyaluronan for a set period of time (30 minutes) and then precipitating the undigested hyaluronan by the addition of CPC. The turbidity of the resulting sample is measured at 640 nm and the decrease in turbidity resulting from enzyme activity on the HA substrate was a measure of the hyaluronidase potency. The method is run using a calibration curve generated with dilutions of rHuPH20 assay working reference standard, and sample activity measurements were made relative to the calibration curve. The method was intended for the analysis of rHuPH20 activity in solutions after dilution to a concentration of ˜2 U/mL. The quantitative range was 0.3 to 3 U/mL, although for routine testing optimum performance was obtained in the range of 1 to 3 U/mL.

Enzyme Diluent was prepared fresh by dissolving 100 mg±10 mg gelatin hydrolysate (Sigma #G0262) in 75 mL of the Reaction Buffer Solution (140 mM NaCl, 50 mM PIPES (1,4 piperazine bis(2-ethanosulfonic acid)), pH 5.3) free acid (Mallinckrodt #V249) and 74.4 mL of Sterile Water for Irrigation (SWFI) and adding 0.6 mL 25% Human Serum Albumin (HSA). A spectrophotometer blank was prepared by adding 1.0 mL Enzyme Diluent to a test tube and placing it in a heating block preheated to 37° C. A Diluted Reference Standard was prepared by making a 1:25 dilution of the rHuPH20 Assay Working Reference Standard in triplicate by adding 120 μL of the Assay Working Reference Standard to 29.880 mL of Enzyme Diluent. Appropriate dilutions of each sample were prepared in triplicate to yield a ˜2 U/mL solution.

The volumes of Enzyme Diluent were dispensed in triplicate into Standard test tubes according to Table 13. 500 μL of a solution of 1.0 mg/mL sodium hyaluronate (Lifecore, #81, with average molecular weight of 20-50 kDa) in SWFI was dispensed into all test tubes except the blank, and the tubes were placed in the 37° C. in the heating block for 5 minutes. The quantity of the Diluted Reference Standard indicated in Table 13 was added to the appropriate Standard test tubes, mixed and returned to the heating block. 500 μL of each sample to the appropriate tubes in triplicate. 30 minutes after the first Standard tube was started, 4.0 mL of Stop Solution (5.0 mg/mL cetylpyridinium chloride (Sigma, Cat #C-5460) dissolved in SWFI and passed through a 0.22 micron filter) to all tubes (including the Blank), which were then mixed and placed at room temperature.

The spectrophotometer was “blanked” at 640 nm fixed wavelength. After 30 minutes incubation at room temperature. Approximately 1 mL of Standard or Sample was transferred to a disposable cuvette and the absorbance read at 640 nm. The Reference Standard and Sample raw data values were analyzed employing GRAPHPAD PRISM® computer software (Hearne Scientific Software) using an exponential decay function constrained to 0 upon complete decay The best fit standard curve was determined and used to calculate the corresponding Sample concentrations.

TABLE 21
Dilutions for Enzyme Standards

| | | Enzyme Diluent | Diluted Reference |
|---|---|---|---|
| Standard | U/mL | (μL) | Standard (μL) |
| 1 | 0.0 | 500 | 0 |
| 2 | 0.6 | 400 | 100 |
| 3 | 1.2 | 300 | 200 |
| 4 | 1.8 | 200 | 300 |
| 5 | 2.4 | 100 | 400 |
| 6 | 3.0 | 0 | 500 |

Since modifications will be apparent to those of skill in this art, it is intended that this invention be limited only by the scope of the appended claims.

1. A method of controlling blood glucose in a subject by continuous subcutaneous insulin infusion (CSII) therapy, comprising:
a) administering a composition comprising a hyaluronan-degrading enzyme to the subject; and then
b) continuously infusing a fast-acting insulin by CSII to the subject, wherein the difference in insulin absorption is minimized or reduced over the course of infusion set life compared to CSII performed in the absence of the hyaluronan-degrading enzyme.

2. The method of claim 1, wherein:
the hyaluronan-degrading enzyme is in an amount that effects an ultra-fast insulin response at the outset of infusion set life in the subject; or
the hyaluronan-degrading enzyme is in an amount sufficient to catalyze the hydrolysis of hyaluronic acid to increase tissue permeability.

3. The method of claim 1, wherein in step b) the continuous infusion is effected by a continuous infusion device that comprises an insulin pump, a reservoir containing the fast-acting insulin, an optional glucose monitor, and an infusion set for subcutaneous infusion of the composition.

4. The method of claim 3, wherein the continuous infusion device comprises an insulin pump, a reservoir containing the fast-acting insulin, a glucose monitor, and an infusion set for subcutaneous infusion of the composition.

5. The method of claim 1, wherein the continuous infusion device provides an open-loop or closed-loop system.

6. The method of claim 1, wherein:
the step b) of continuously infusing a fast-acting insulin by CSII continues for a predetermined interval; and
at the end of each interval steps a) and b) are repeated.

7. The method of claim 6, wherein after the end of each interval the infusion set is replaced.

8. The method of claim 6, wherein each predetermined interval is more than a day or is several days.

9. The method of claim 8, wherein several days is 2 days to 4 days.

10. The method of claim 1, wherein in step a) the hyaluronan-degrading enzyme is administered at or near the site of infusion of the composition in step b).

11. The method of claim 1, wherein the hyaluronan-degrading enzyme in step a) and the fast-acting insulin composition in step b) are administered through the same injection site.

12. The method of claim 1, wherein the hyaluronan-degrading enzyme in step a) and the fast-acting insulin composition in step b) are administered through different injection sites.

13. The method of claim 1, wherein the hyaluronan-degrading enzyme is administered 1 minute to 12 hours, 5 minutes to 6 hours, 30 minutes to 3 hours, 1 hour to 2 hours, 15 seconds to 1 hour, 30 seconds to 30 minutes or 1 minute to 15 minutes prior to infusion of the fast-acting insulin.

14. The method of claim 1, wherein the hyaluronan-degrading enzyme in step a) is administered no more than 2 hours before infusion of the fast-acting insulin.

15. The method of claim 1, wherein the hyaluronan-degrading enzyme is a hyaluronidase.

16. The method of claim 1, wherein the hyaluronan-degrading enzyme is a hyaluronidase that is active at neutral pH.

17. The method of claim 1, wherein:
the hyaluronan-degrading enzyme lacks a glycosylphosphatidylinositol (GPI) anchor or is not membrane-associated when expressed from a cell; or
the hyaluronan-degrading enzyme contains C-terminal truncations of one or more amino acid residues to remove all or part of a GPI anchor.

18. The method of claim 1, wherein the hyaluronan-degrading enzyme is a hyaluronidase that is a PH20 or a C-terminally truncated fragment thereof.

19. The method of claim 1, wherein the hyaluronan-degrading enzyme is a PH20 polypeptide that has the sequence of amino acids set forth in any of SEQ ID NOS: 4-9, 47-48, 234-254, and 267-273, or a sequence of amino acids that exhibits at least 70%, 75%, 80%, 85%, 86%, 87%, 88%, 89%, 90%, 91%, 92%, 93%, 94%, 95%, 96%, 97%, 98%, 99% or more sequence identity to any of SEQ ID NOS: 4-9, 47-48, 234-254, and 267-273.

20. The method of claim 1, wherein the hyaluronan-degrading enzyme is a C-terminal truncated PH20 that comprises a sequence of amino acids set forth in any of SEQ ID NOS: 4-9.

21. The method of claim 1, wherein:
the hyaluronan-degrading enzyme in step a) is administered in an amount that is functionally equivalent to between or about between 1 Unit to 200 Units, 5 Units to 150 Units, 10 Units to 150 Units, 50 Units to 150 Units or 1 Unit to 50 Units; or
the hyaluronan-degrading enzyme in step a) is administered in an amount that is between or about between 8 ng to 2 μg, 20 ng to 1.6 μg, 80 ng to 1.25 μg or 200 ng to 1 μg.

22. The method of claim 1, wherein the fast-acting insulin is a regular insulin.

23. The method of claim 22, wherein the regular insulin is a human insulin or pig insulin.

24. The method of claim 22, wherein the regular insulin is an insulin with an A chain having a sequence of amino acids set forth in SEQ ID NO:103 and a B chain having a sequence of amino acids set forth in SEQ ID NO:104 or an insulin with an A chain with a sequence of amino acids set forth as amino acid residue positions 88-108 of SEQ ID NO:123 and a B chain with a sequence of amino acids set forth as amino acid residue positions 25-54 of SEQ ID NO:123.

25. The method of claim 1, wherein the fast-acting insulin is an insulin analog.

26. The method of claim 25, wherein the fast-acting insulin is an insulin analog selected from among insulin lispro, insulin aspart or insulin glulisine.

27. The method of claim 25, wherein the insulin analog is selected from among an insulin having an A chain with a sequence of amino acids set forth in SEQ NOS:103 and a B chain having a sequence of amino acids set forth in any of SEQ NOS:147-149.

28. The method of claim 1, wherein:
the insulin composition comprises insulin in an amount that is between or about between 10 U/mL to 1000 U/mL; or
the insulin composition comprises insulin in an amount that is between or about between 0.35 mg/mL to 35 mg/mL.

29. The method of claim 1, wherein the insulin in the composition is at least or is about at least or is 100 U/mL.

30. The method of claim 1, wherein the composition in step b) comprises a fast-acting insulin analog and a hyaluronan-degrading enzyme.

31. The method of claim 30, wherein:
the amount of fast-acting insulin analog in the composition is between or about between 10 U/mL to 1000 U/mL; and
the amount of a hyaluronan-degrading enzyme in the composition is functionally equivalent to between or about between 1 U/mL to 10,000 U/mL.

32. The method of claim 1, wherein the subject has diabetes.

33. The method of claim 32, wherein the diabetes is selected from among type 1 diabetes mellitus, type 2 diabetes mellitus and gestational diabetes.

34. A continuous subcutaneous insulin infusion (CSII) dosage regimen method for controlling blood glucose in a subject, comprising:
a) performing CSII to deliver a composition comprising a super fast-acting insulin composition to a subject in accord with a programmed basal rate and bolus dose of insulin; and
b) at least once during the course of treatment, increasing the amount of basal insulin and/or bolus insulin administered by at least 1% compared to the programmed basal rate and bolus dose of insulin administered in the absence of a hyaluronan-degrading enzyme thereby increasing insulin action.

35. The method of claim 34, wherein the basal insulin and/or bolus insulin administered are increased at least once per day.

36. The method of claim 34, wherein the super-fast acting insulin composition comprises:
a therapeutically effective amount of a fast-acting insulin analog for controlling blood glucose levels; and
an amount of a hyaluronan-degrading enzyme sufficient to render the composition a super fast-acting insulin composition.

37. The method of claim 36, wherein:
the amount of fast-acting insulin is from or from about 10 U/mL to 1000 U/mL; and the sufficient amount of a hyaluronan-degrading enzyme to render the composition super fast-acting is functionally equivalent to 1 U/mL to 10,000 U/mL; or
the amount of fast-acting insulin is from or from about 0.35 mg/mL to 35 mg/mL; and
the sufficient amount of a hyaluronan-degrading enzyme to render the composition super fast-acting is functionally equivalent to 8 ng/mL to 80 μg/mL.

38. The method of claim 34, wherein the subject has diabetes.

39. The method of claim 38, wherein the diabetes is selected from among type 1 diabetes mellitus, type 2 diabetes mellitus and gestational diabetes.
